(19) United States
(12) Patent Application Publication
(10) Pub. No.: US 2021/0330646 A1
(43) Pub. Date: Oct. 28, 2021

(54) FASCIN BINDING COMPOUNDS FOR SPINOGENESIS

(71) Applicant: SPINOGENIX, INC., San Diego, CA (US)

(72) Inventors: Stella T. SARRAF, Beverly Hills, CA (US); Vincent F. SIMMON, San Diego, CA (US); Peter W. VANDERKLISH, San Diego, CA (US)

(21) Appl. No.: 17/271,163

(22) PCT Filed: Aug. 27, 2019
(86) PCT No.: PCT/US19/48408

§ 371 Date: Feb. 24, 2021

Related U.S. Application Data
(60) Provisional application No. 62/723,381, filed on Aug. 27, 2018.
(60) Provisional application No. 62/726,904, filed on Sep. 4, 2018.
(60) Provisional application No. 62/785,435, filed on Dec. 27, 2018.

(51) Int. Cl. A61K 31/426; A61P 25/28; A61K 31/416

(52) U.S. Cl. CPC A61K 31/426; A61K 31/416; A61P 25/28

(57) ABSTRACT

In some embodiments, a method of promoting spinogenesis in a patient is provided, comprising administering to a patient in need thereof a therapeutically effective amount of a compound which binds to fascin at least at binding site 2 or binding site 3.

### CROSS-REFERENCE TO RELATED PATENT APPLICATIONS

This application claims the benefit under 35 U.S.C. § 119(e) of United States Provisional Application Nos. 62/723,381, filed Aug. 27, 2018, 62/726,904, filed Sep. 4, 2018, and 62/785,435, filed Dec. 27, 2018, each of which is hereby incorporated by reference in its entirety.

### FIELD

Provided herein are methods for promoting spinogenesis and for treating a neuronal disease or disorder.

### BACKGROUND

Neurological disorders are diseases of the brain, spinal cord and peripheral nervous system. The greatest societal costs, in terms of epidemiology and individual morbidity, are imposed by neurodegenerative conditions, which result in the damage or loss of neurons and the synaptic connections between them. Among the most prominent of these are Alzheimer's disease and Parkinson's disease. Other neurodegenerative conditions include age-related conditions (e.g. Parkinson's dementia, vascular dementia, Amyotrophic lateral sclerosis), genetic syndromes (e.g. Down syndrome), injury-related conditions (e.g. Traumatic Brain Injury, Chronic Traumatic Encephalopathy), and conditions typically considered as being purely psychiatric in nature, such as schizophrenia and depression.

Researchers have classified hundreds of diseases of the nervous system, such as brain tumors, epilepsy, Alzheimer's disease, Parkinson's disease and stroke, as well as conditions associated with old age, such as dementia. Some such conditions result from a progressive loss of synapses (junctions between two different neurons) and ultimately a loss of neurons (neurodegeneration). Unfortunately, neurodegenerative diseases have been almost completely resistant to treatment. Neurons in the brain communicate with each other by the sending neuron releasing chemicals (neurotransmitters) into the synapse, altering the electrical potential of the receiving neuron. The part of a neuron that releases the neurotransmitter is the axon (the presynaptic side of a synapse) and the part of the synapse that is affected by neurotransmitter is called a dendritic spine (the postsynaptic side of a synapse). Changes in the number, location and even shape of synaptic junctions underlie memory, learning, thinking and our personality. A part of the brain called the hippocampus is intimately involved in the formation of memories, and suffers from a notable loss of synapses and neurons in neurodegenerative diseases. The development of novel methods to restore spine density in the hippocampus could have important implications for treatment of a host of neurodegenerative and developmental cognitive disorders.

Dendritic complexity, synaptogenesis, and proper development and function of neurons are endogenously regulated by growth factors such as brain derived neurotrophic factor (BDNF). While some small molecules have recently been reported to exhibit neurotrophic-like activity, these molecules have not been demonstrated to promote dendritic spine formation. The identification of a new cellular target for small molecules could lead to treatments for many neurodegenerative and mental development disorders, and also have the potential for providing improved memory and learning. Thus, small molecules that promote spine formation have potential use in ameliorating cognitive deficiencies in neurodegenerative diseases such as Alzheimer's disease, and might also find use as general cognitive enhancers. However, there is a need for pharmaceutically acceptable compounds having such activity.

### SUMMARY

Provided herein are methods for promoting spinogenesis and for treating a neuronal disease or disorder.

In some embodiments, a method of promoting spinogenesis or treating a neuronal disease or disorder in a patient is provided, comprising contacting fascin with an agent which inhibits the activity of fascin.

In some embodiments, a method of promoting spinogenesis in a patient is provided, comprising administering to a patient in need thereof a therapeutically effective amount of a compound which binds to fascin at least at binding site 2 or binding site 3.

In some embodiments, a method of promoting spinogenesis in a patient is provided, comprising administering to a patient in need thereof a therapeutically effective amount of a compound which binds to fascin at least at binding site 2.

In some embodiments, a method of promoting spinogenesis in a patient is provided, comprising administering to a patient in need thereof a therapeutically effective amount of a compound which binds to fascin at least at binding site 3.

In some embodiments, a method of promoting spinogenesis in a patient is provided, comprising administering to a patient in need thereof a therapeutically effective amount of a compound of formula I:

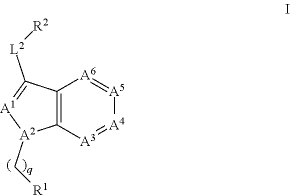

or a pharmaceutically acceptable salt thereof;

wherein A¹, A², A³, A⁴, A⁵ and A⁶ are independently selected from the group consisting of CH, CR³ and N, provided that no more than four of A¹, A², A³, A⁴, A⁵ and A⁶ are N;

R¹ is selected from the group consisting of phenyl, 5-membered heteroaryl and 6-membered heteroaryl, wherein the phenyl, 5-membered heteroaryl or 6-membered heteroaryl is optionally substituted with 1 to 3 R⁶;

L² is selected from the group consisting of a covalent bond, —NR⁸—, —C(O)NR⁸—, —NR⁸—, —C(O)NR⁸—, —NR⁸C(O)—, —C(O)CR⁸₂—, —CR⁸₂C(O)—, —NR⁸CR⁸₂—, and —CR⁸₂NR⁸—;

R² is H, C₁₋₆ alkyl, 6- to 10-membered aryl or 5- to 10-membered heteroaryl; wherein the 6- to 10-membered aryl or 5- to 10-membered heteroaryl is optionally substituted with 1 to 4 R⁴, wherein each R⁴ is independently selected from the group consisting of C₁₋₆ alkyl, C₁₋₆ haloalkyl, phenyl (optionally substituted with C₁₋₆ alkyl, halo, C₁₋₆ haloalkyl, or —OH), —OH, —OR⁷, —SH, —SR⁷, —NR¹⁰R¹⁰, halo, cyano, nitro, —COH, —COR⁷, —CO₂H, —CO₂R⁷, —CONR¹⁰R¹⁰, —OCOR⁷, —OCO₂R⁷, —OCONR¹⁰R¹⁰, —NR¹⁰COR⁷, —NR¹⁰CO₂R⁷, —SOR⁷, —SO₂R⁷, —SO₂NR¹⁰R¹⁰, and —NR¹⁰SO₂R⁷;

each R³ is independently selected from the group consisting of C₁₋₆ alkyl, C₁₋₆ haloalkyl, —OH, —OR⁷, —SH, —SR⁷, —NR¹⁰R¹⁰, halo, cyano, nitro, —COH, —COR⁷, —CO₂H, —CO₂R⁷, —CONR¹⁰R¹⁰, —OCOR⁷, —OCO₂R⁷, —OCONR¹⁰R¹⁰, —NR¹⁰COR⁷, —NR¹⁰CO₂R⁷, —SOR⁷, —SO₂R⁷, —SO₂NR¹⁰R¹⁰, and —NR¹⁰SO₂R⁷;

q is 1, 2 or 3;

each R⁶ is independently selected from the group consisting of cyano, halo, C₁₋₆ alkyl, C₁₋₆ haloalkyl, and —CH₂OH;

R⁷ is C₁₋₆ alkyl or C₁₋₆ haloalkyl;

R⁸ is hydrogen or C₁₋₆ alkyl;

each R¹⁰ is independently hydrogen or C₁₋₆ alkyl, or two R¹⁰ together with the atom(s) attached thereto form a 4- to 6-membered ring; and

R¹¹ is hydrogen or R³.

In some embodiments, a method of treating depression is provided, comprising administering to a patient in need thereof a therapeutically effective amount of a compound of formula I, or a pharmaceutically acceptable salt thereof.

In some embodiments, the compound is a compound described in US Patent Publication No. 2015/0299191. In some embodiments, the compound is a compound described in US Patent Publication No. 2014/0080843.

In some embodiments, the compound is a compound described in International Patent Publication No. WO 2013/013240. In some embodiments, the compound is a compound described in US Patent Publication No. 2014/0024705.

In some embodiments, a method of treating or preventing a neuronal disease or disorder is provided, comprising administering to a patient in need thereof, a therapeutically effective amount of a compound which binds to fascin at least at binding site 2.

In some embodiments, a method of treating or preventing a neuronal disease or disorder is provided, comprising administering to a patient in need thereof, a therapeutically effective amount of a compound which binds to fascin at least at binding site 3.

In some embodiments, a method of treating or preventing a neuronal disease or disorder is provided, comprising administering to a patient in need thereof a therapeutically effective amount of a compound described herein, for example, a compound selected from a compound of formula I as provided herein, or a pharmaceutically acceptable salt thereof, a compound of formula II as provided herein, or a pharmaceutically acceptable salt thereof, a compound selected from a compound of formula IV as provided herein, or a pharmaceutically acceptable salt thereof, a compound selected from a compound of formula V as provided herein, or a pharmaceutically acceptable salt thereof, a compound selected from a compound selected from a compound of formula VII as provided herein, or a pharmaceutically acceptable salt thereof, or compound 1, compound 8, compound 9, compound 10, or compound 11 or a pharmaceutically acceptable salt thereof.

In some embodiments, the neuronal disease or disorder is selected from Alzheimer's disease, Parkinson's disease, Parkinson's dementia, autism, fragile X syndrome, and traumatic brain injury. In some embodiments, the neuronal disease or disorder is selected from Alzheimer's disease, Parkinson's disease, Parkinson's dementia, autism, fragile X syndrome, depression and traumatic brain injury.

In some embodiments, the neuronal disease or disorder is a mood disorder, for example, depression.

In some embodiments, provided herein is a method of promoting spinogenesis in a patient, comprising administering to a patient in need thereof a therapeutically effective amount of an agent described herein, for example, a compound selected from a compound of formula II, formula IV, formula V, formula VII, formula VIII, formula IX, formula X, or formula XI, or a pharmaceutically acceptable salt thereof, or compound 1, compound 8, compound 9, compound 10, or compound 11, or a pharmaceutically acceptable salt thereof.

In some embodiments, a method of promoting spinogenesis in a patient is provided, comprising administering to a patient in need thereof a therapeutically effective amount of N-(1-(4-(trifluoromethyl)benzyl)-1H-indazol-3-yl)furan-2-carboxamide (compound 1), having the structure:

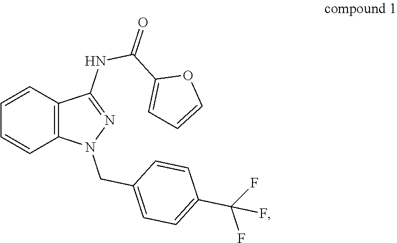 [SMILES: O=C(Nc1nn(Cc2ccc(C(F)(F)F)cc2)c2ccccc12)c1ccco1]

or a pharmaceutically acceptable salt thereof.

In some embodiments, a compound that inhibits fascin is provided, wherein the compound does not bind to fascin at binding site 1.

In some embodiments, a method of promoting spinogenesis in a patient is provided, comprising administering to a patient in need thereof a therapeutically effective amount of a compound which binds to fascin, provided the compound is not a compound of formula III:

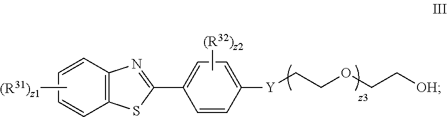

wherein Y is —NR³³—, O, or —S—; R³¹ is independently halogen, —CX³¹, —CHX³¹, —CH₂X³¹, —OCX³¹₃, —OCHX³¹₂, —OCH₂X³¹, —CN, —OH, —NH₂, —COOH, —CONH₂, —NO₂, —SH, —SO₃H, —SO₄H, —SO₂NH₂, —NHNH₂, —ONH₂, —HNC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocyclyl, substituted or unsubstituted aryl, or substituted or unsubstituted heteroaryl; R³² is independently halogen, —CX³²₃, —CHX³²₂, —CH₂X³², —OCX³—OCHX³²₂, —OCH₂X³², —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocyclyl, substituted or unsubstituted aryl, or substituted or unsubstituted heteroaryl; each of X³¹ and X³² are independently halogen; each of z1 and z2 is independently an integer from 0 to 4; z3 is an integer from 1 to 12; and R³³ is hydrogen or substituted or unsubstituted C₁₋₆ alkyl. In some embodiments, of formula III, Y is —NR³³— or —S—.

### BRIEF DESCRIPTION OF THE DRAWINGS

FIG. 1 illustrates the three known actin binding sites of fascin in an embodiment.

FIGS. 2A-2D provide front, bottom, top, and back views of fascin, respectively, based on available crystal structures in an embodiment.

FIGS. 3A-3C illustrate docked complexes of human fascin 1 and comparative compound 2, comparative compound 3, and comparative compound 4 in an embodiment

FIG. 4 illustrates a 2D interaction diagram of the comparative compound 2 and human fascin 1 complex in an embodiment.

FIG. 5 illustrates a fascin apo structure superimposed on the compound 1 bound fascin structure wherein binding site 1 is as shown and binding site 2 is as shown in an embodiment.

FIG. 6 illustrates the results of synaptic growth for comparative compound 2, comparative compound 7, and compound 1 compared to vehicle control in an embodiment.

### DETAILED DESCRIPTION

Generally the compositions and methods described herein provide for the administration of compounds that bind fascin at binding site 2 or binding site 3 for promoting spinogenesis. In some embodiments, the compositions and methods are useful for treating a neuronal disease or disorder. Generally, the active ingredient or principal ingredient will include an agent, such as a compound or a pharmaceutically acceptable salt of a compound as described herein. The active ingredient or principal ingredient may also include one or more additional pharmaceutically active materials.

### I. Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

The term “fascin” refers to a 54-58 kDa protein that is an actin cross-linking protein. The term “fascin” may refer to the amino acid sequence of human fascin 1. The term “fascin” includes both the wild-type form of the nucleotide sequences or proteins as well as any mutants thereof. In some embodiments, “fascin” is wild-type fascin. In some embodiments, “fascin” is one or more mutant forms. In some embodiments, a fascin is human fascin 1. In some embodiments, the fascin protein is encoded in the nucleotide sequence corresponding to reference number GT347360903. In some embodiments, the fascin protein is encoded in the nucleotide sequence of RefSeq M_003088. In some embodiments, the fascin corresponds to the amino acid sequence of RefSeq NP_003079.1.

The term “spinogenesis” and the like refer, in the usual and customary sense, to development (e.g. growth and/or maturation) of dendritic spines in neurons. In some embodiments, the compounds provided herein promote spinogenesis without affecting spine morphology. The promotion is relative to the absence of administration of the compound.

As used herein, the term “dendrite” refers to the branched extension of a neuron cell. Dendrites are typically responsible for receiving electrochemical signals transmitted from the axon of an adjacent neuron. The terms “dendritic spines” or “dendrite spines” refer to protoplasmic protuberances on a neuron cell (e.g., on a dendrite). In some embodiments, dendritic spines may be described as having a membranous neck which may be terminated with a capitulum (e.g., head). Dendritic spines are classified according to their shape: headless, thin, stubby, mushroom, or branched. Dendritic spine density refers to the total number of dendritic spines per unit length of a neuron cell. For example, the dendritic spine density may be given as the number of dendritic spines per micron.

The term “dendritic spine formation” and the like refer, in the usual and customary sense to processes which lead to an increased number of dendritic spines or increased development of dendritic spines. The term “dendritic spine morphology” and the like refer, in the usual and customary sense, to physical characterization of a dendritic spine (e.g., shape and structure). Improvement of dendritic spine morphology is a change in morphology (e.g., increase in length or increase in width) that results in increased functionality (e.g., increased number of contacts between neurons or decreased space between neighboring neurons (e.g., synaptic cleft)). As known in the art and disclosed herein, exemplary methods for such characterization include measurement of the dimensions (i.e., length and width) of dendritic spines. Accordingly, the term “improving dendritic spine morphology” generally refers to an increase in length, width, or both length and width of a dendritic spine.

“Binding” refers to at least two distinct species (e.g. chemical compounds including biomolecules, or cells) to becoming sufficiently proximal to react or interact thereby resulting in the formation of a complex. For example, the binding of two distinct species (e.g., a protein and a compound described herein) may result in the formation of a complex wherein the species are interacting via non-covalent or covalent bonds. In some embodiments, the resulting complex is formed when two distinct species (e.g., a protein and a compound described herein) interact via non-covalent bonds (e.g., electrostatic, van der Waals, or hydrophobic).

As defined herein, the term “activation,” “activate,” “activating” and the like in reference to a protein-activator (e.g. agonist) interaction means positively affecting (e.g. increasing) the activity or function of the protein relative to the activity or function of the protein in the absence of the activator (e.g. compound described herein).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g. chemical compounds including biomolecules, or cells) to become sufficiently proximal to interact. The term “contacting” may include allowing two molecular species to react or physically touch, wherein the two species may be, for example, a compound as described herein, a biomolecule, a protein or an enzyme. In some embodiments contacting includes allowing a compound described herein to interact with a protein (e.g., fascin) or enzyme. In some embodiments, contacting may comprise binding a protein.

As defined herein, the terms “inhibition,” “inhibit,” “inhibiting” and the like, are to be given their customary meanings to those of skill in the art. In reference to a protein-inhibitor (e.g. antagonist) interaction, the terms “inhibition,” “inhibit,” “inhibiting” mean negatively affecting (e.g. decreasing) the functional activity of the protein relative to the functional activity of the protein in the absence of the inhibitor.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e. —CH(CH₃)CH₂CH₃), isobutyl (i.e. —CH₂CH(CH₃)₂) and tert-butyl (i.e. —C(CH₃)₃); and “propyl” includes n-propyl (i.e. —(CH₂)₂CH₃) and isopropyl (i.e. —CH(CH₃)₂). In some embodiments, the term “lower alkyl” refers to a C₁₋₆ alkyl.

“Alkenyl” refers to an alkyl group containing at least one carbon-carbon double bond and having from 2 to 20 carbon atoms (i.e., C₂₋₂₀ alkenyl), 2 to B carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

“Alkynyl” refers to an alkyl group containing at least one carbon-carbon triple bond and having from 2 to 20 carbon atoms (i.e., C₂₋₂₀ alkynyl), 2 to 8 carbon atoms (i.e., C₂₋₅ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include methoxy, ethoxy, n-propoxy, iso-propoxy, n-butoxy, tert-butoxy, sec-butoxy, n-pentoxy, n-hexoxy, and 1,2-dimethylbutoxy.

“Haloalkoxy” refers to an alkoxy group as defined above, wherein one or more hydrogen atoms are replaced by a halogen.

“Alkylthio” refers to the group “alkyl-S—”.

“Acyl” refers to a group —C(O)R, wherein R is hydrogen, alkyl, cycloalkyl, heterocyclyl, aryl, heteroalkyl, or heteroaryl; each of which may be optionally substituted, as defined herein. Examples of acyl include formyl, acetyl, cyclohexylcarbonyl, cyclohexylmethyl-carbonyl, and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently selected from the group consisting of hydrogen, alkyl, aryl, haloalkyl, or heteroaryl; each of which may be optionally substituted.

“Amino” refers to the group —NR^{y}R^{z} wherein R^{y} and R^{z} are independently selected from the group consisting of hydrogen, alkyl, haloalkyl, aryl, or heteroaryl; each of which may be optionally substituted.

“Aryl” refers to an aromatic carbocyclic group having a single ring (e.g. monocyclic) or multiple rings (e.g. bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include phenyl, naphthyl, fluorenyl, and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Aralkyl” refers to an aryl group pendant to an alkyl group. Examples of aralkyl groups include benzyl, phenethyl, and 3-naphthylpropyl.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently selected from the group consisting of hydrogen, alkyl, aryl, haloalkyl, or heteroaryl; each of which may be optionally substituted.

“Carboxyl” refers to —C(O)OH.

“Carboxyl ester” refers to both —OC(O)R and —C(O)OR, wherein R is hydrogen, alkyl, cycloalkyl, heterocyclyl, aryl, heteroalkyl, or heteroaryl; each of which may be optionally substituted, as defined herein.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e. the cyclic group having at least one double bond). As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₅ cycloalkyl), or 3 to 6 ring carbon atoms (i.e., C₃₋₆ cycloalkyl). Examples of cycloalkyl groups include cyclopropyl, cyclobutyl, cyclopentyl, and cyclohexyl, and partially unsaturated groups such as cyclopentenyl and cyclohexenyl.

“Imino” refers to a group —C(NR)R, wherein each R is alkyl, cycloalkyl, heterocyclyl, aryl, heteroalkyl, or heteroaryl; each of which may be optionally substituted, as defined herein.

“Halogen” or “halo” includes fluoro, chloro, bromo, and iodo. “Haloalkyl” refers to an unbranched or branched alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen Examples of haloalkyl include difluoromethyl (—CHF₂) and trifluoromethyl (—CF₃).

“Heteroalkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR—, —O—, —S—, —S(O)—, —S(O)₂—, and the like, where R is H, alkyl, aryl, cycloalkyl, heteroalkyl, heteroaryl or heterocyclyl, each of which may be optionally substituted. Examples of heteroalkyl groups include —OCH₃, —CH₂OCH₃, —SCH₃, —CH₂SCH₃, —NRCH₃, and —CH₂NRCH₃, where R is hydrogen, alkyl, aryl, arylalkyl, heteroalkyl, or heteroaryl, each of which may be optionally substituted. As used herein, heteroalkyl include 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to an aromatic group having a single ring, multiple rings, or multiple fused rings, with one or more ring heteroatoms independently selected from nitrogen, oxygen, and sulfur. As used herein, heteroaryl includes 5 to 20 ring atoms (i.e., 5 to 20 membered heteroaryl), or 5 to 10 ring atoms (i.e., 5 to 10 membered heteroaryl); and 1 to 5 heteroatoms, 1 to 4 heteroatoms, 1 to 3 ring heteroatoms, 1 to 2 ring heteroatoms, or 1 ring heteroatom independently selected from nitrogen, oxygen, and sulfur. Examples of heteroaryl groups include pyrimidinyl, purinyl, pyridyl, pyridazinyl, benzothiazolyl, and pyrazolyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” and “heterocycloalkyl” refer to a saturated or unsaturated cyclic alkyl group, with one or more ring heteroatoms independently selected from nitrogen, oxygen and sulfur. The term “heterocyclyl” and “heterocycloalkyl” include heterocycloalkenyl groups (i.e. the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 3 to 20 ring atoms (i.e., 3 to 20 membered heterocyclyl), 3 to 12 ring atoms (i.e., 3 to 12 membered heterocyclyl), or 3 to 10 ring atoms (i.e., 3 to 10 membered heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. Examples of heterocyclyl groups include pyrrolidinyl, piperidinyl, piperazinyl, oxetanyl, dioxolanyl, azetidinyl, and morpholinyl. As used herein, the term “bridged-heterocyclyl” refers to a four- to ten-membered cyclic moiety connected at two non-adjacent atoms of the heterocyclyl with one or more (e.g. 1 or 2) four- to ten-membered cyclic moiety having at least one heteroatom where each heteroatom is independently selected from nitrogen, oxygen, and sulfur. As used herein, bridged-heterocyclyl includes bicyclic and tricyclic ring systems. Also used herein, the term “spiro-heterocyclyl” refers to a ring system in which a three- to ten-membered heterocyclyl has one or more additional ring, wherein the one or more additional ring is three- to ten-membered cycloalkyl or three- to ten-membered heterocyclyl, where a single atom of the one or more additional ring is also an atom of the three- to ten-membered heterocyclyl. Examples of the spiro-heterocyclyl rings include bicyclic and tricyclic ring systems, such as 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

“Oxo” refers to the group (═O) or (O).

“Sulfonyl” refers to the group —S(O)₂R, where R is alkyl, haloalkyl, heterocyclyl, cycloalkyl, heteroaryl, or aryl. Examples of sulfonyl are methyl sulfonyl, ethyl sulfonyl, phenyl sulfonyl, and toluenesulfonyl.

“Alkylsulfonyl” refers to the group —S(O)₂R, where R is alkyl.

“Alkylsulfinyl” refers to the group —S(O)R, where R is alkyl.

“Thiocyanate” refers to the group —SCN.

“Thioxo” or “thione” refer to the group (═S) or (S).

As used herein, the term “saccharide” refers to a sugar, such as a monosaccharide, a disaccharide, an oligosaccharide or a polysaccharide. Monosaccharides include, but are not limited to, glucose, ribose and fructose. Disaccharides include, but are not limited to, sucrose and lactose. Oligosaccharides refers to 2 to 10 sugars linked together preferably through an alpha linkage. Examples of oligosaccharides include maltose, lactose, sucrose, and the like. Polysaccharides include, but are not limited to, cellulose, hemicellulose and lignocellulose or starch. Saccharides or sugars may include any and all naturally occurring sugars, such as, but not limited to, glucose, glucuronic acid, iduronic acid, galactose, fucose, glucosamine, N-acetylglucosamine, fructose, sialic acid, including aldol and pyranose forms thereof, as well as D and L isomers thereof.

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g. arylalkyl, the last mentioned group contains the atom by which the moiety is attached to the rest of the molecule.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen.

A protecting group may be any known in the art, for example, as described in Peter G. M. Wuts and Theodora W. Greene, Greene's protective groups in organic synthesis (Wiley-Interscience, 2007). In some embodiments, an oxygen protecting group may be selected from a benzyl ether, a silyl ether, an ester, a carbonate, a cyclic acetal and a ketal.

Some compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise all tautomers.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure, or counter-ions thereto, include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds are possible under the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of compounds, and counter-ions thereto, in which from 1 to n hydrogens attached to a carbon atom is/are replaced by deuterium, in which n is the number of hydrogens in the molecule. Such compounds exhibit increased resistance to metabolism and are thus useful for increasing the half-life of a compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

Compounds described herein may be present as a salt, such as a pharmaceutically acceptable salt. Compounds are capable of forming salts such as acid and/or base salts. Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use. Salts of compounds described herein can be prepared according to procedures described herein and as known in the art.

The term “pharmaceutically acceptable salt” of a given compound, refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri (substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like. Methods of preparing a salt also include mixing a compound by redox reaction with an active metal, or by exchange of ions, for example, due to differing solubility of salts.

The term “substituted” means that any one or more hydrogen atoms on the designated atom or group is replaced with one or more substituents other than hydrogen, provided that the designated atom's normal valence is not exceeded. The one or more substituents include, but are not limited to, alkyl, alkenyl, alkynyl, alkoxy, acyl, amino, amido, aryl, —N₃, carbamoyl, carboxyl, carboxyl ester, —CN, halo, haloalkyl, haloalkoxy, heteroalkyl, heteroaryl, heterocyclyl, —OH, imino, oxo, —NO₂, alkylsulfinyl, —SO₃H, alkylsulfonyl, thiocyanate, —SH, thione, or combinations thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. Unless specified otherwise, where a group is described as optionally substituted, any substituents of the group are themselves unsubstituted. For example, in some embodiments, the term “substituted alkyl” refers to an alkyl group having one or more substituents including hydroxyl, halo, alkoxy, cycloalkyl, heterocyclyl, aryl, and heteroaryl. In other embodiments, the one or more substituents may be further substituted with halo, alkyl, haloalkyl, hydroxyl, alkoxy, cycloalkyl, heterocyclyl, aryl, or heteroaryl, each of which is substituted. In other embodiments, the substituents may be further substituted with halo, alkyl, haloalkyl, alkoxy, hydroxyl, cycloalkyl, heterocyclyl, aryl, or heteroaryl, each of which is unsubstituted.

A “solvate” is a solid form of a compound in which solvent molecules are incorporated. A solvate is formed by the interaction of a solvent and a compound. A hydrate is a solvate in which the solvent is water. Solvates of salts of compounds described herein are also provided.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Treatment” or “treating” is an approach for obtaining beneficial or desired results including clinical results. Beneficial or desired clinical results may include one or more of the following: a) treating the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human. When the subject is a human person, the subject may be referred to as a “patient.”

The term “therapeutically effective amount” or “effective amount” of a compound described herein or a pharmaceutically acceptable salt thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a neuronal disease. The therapeutically effective amount may vary depending on the subject, and disease or condition being treated, the weight and age of the subject, the severity of the disease or condition, and the manner of administering, which can readily be determined by one or ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

Table of Amino Acids and Abbreviations

| Amino acid | | |
|---|---|---|
| Alanine | ala | A |
| Cysteine | cys | C |
| Aspartic acid | asp | D |
| Glutamic acid | glu | E |
| Phenylalanine | phc | F |
| Glycine | gly | G |
| Histidine | his | H |
| Isoleucine | ile | I |
| Lysine | lys | K |
| Leucine | leu | L |
| Methionine | met | M |
| Asparagine | asn | N |
| Proline | pro | P |
| Glutamine | gln | Q |
| Arginine | arg | R |
| Serine | ser | S |
| Threonine | thr | T |
| Valine | val | V |
| Tryptophan | trp | W |
| Tyrosine | tyr | Y |

### Compounds

Provided herein are agents that promote spinogenesis. Such agents are useful in the treatment of neuronal diseases and disorders. The agent may be a compound provided herein. The compound may be a compound described in U.S. Patent Publication No. 2015/0299191. The compound may be a compound of formula I:

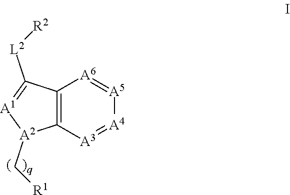

or a pharmaceutically acceptable salt thereof;
wherein A¹, A², A³, A⁴, A⁵ and A⁶ are independently selected from the group consisting of CH, CR³ and N, provided that no more than four of A¹, A², A³, A⁴, A⁵ and A⁶ are N;
R¹ is selected from the group consisting of phenyl, 5-membered heteroaryl and 6-membered heteroaryl, wherein the phenyl, 5-membered heteroaryl or 6-membered heteroaryl is optionally substituted with 1 to 3 R⁶;
L² is selected from the group consisting of a covalent bond, —NR⁸—, —C(O)NR⁸—, —NR⁸—, —C(O)NR⁸—, —NR⁸C(O)—, —C(O)CR⁸₂—, —CR⁸₂C(O)—, —NR⁸CR⁸₂—, and —CR⁸₂NR⁸—;
R² is H, C₁₋₆ alkyl, 6- to 10-membered aryl or 5- to 10-membered heteroaryl; wherein the 6- to 10-membered aryl or 5- to 10-membered heteroaryl is optionally substituted with 1 to 4 R⁴, wherein each R⁴ is independently selected from the group consisting of C₁₋₆ alkyl, C₁₋₆ haloalkyl, phenyl (optionally substituted with C₁₋₆ alkyl, halo, C₁₋₆ haloalkyl, or —OH), —OH, —OR⁷, —SH, —SR⁷, —NR¹⁰R¹⁰, halo, cyano, nitro, —COH, —COR⁷, —CO₂H, —CO₂R⁷, —CONR¹⁰R¹⁰, —OCOR⁷, —OCO₂R⁷, —OCONR¹⁰R¹⁰, —NR¹⁰COR⁷, —NR¹⁰CO₂R⁷, —SOR⁷, —SO₂R⁷, —SO₂NR¹⁰R¹⁰, and —NR¹⁰SO₂R⁷;
each R³ is independently selected from the group consisting of C₁₋₆ alkyl, C₁₋₆ haloalkyl, —OH, —OR⁷, —SH, —SR⁷, —NR¹⁰R¹⁰, halo, cyano, nitro, —COH, —COR⁷, —CO₂H, —CO₂R⁷, —CONR¹⁰R¹⁰, —OCOR⁷, —OCO₂R⁷, —OCONR¹⁰R¹⁰, —NR¹⁰COR⁷, —NR¹⁰CO₂R⁷, —SOR⁷, —SO₂R⁷, —SO₂NR¹⁰R¹⁰, and —NR¹⁰SO₂R⁷;
q is 1, 2 or 3;
each R⁶ is independently selected from the group consisting of cyano, halo, C₁₋₆ alkyl, C₁₋₆ haloalkyl, and —CH₂OH;
R⁷ is C₁₋₆ alkyl or C₁₋₆ haloalkyl;
R⁸ is hydrogen or C₁₋₆ alkyl;
each R¹⁰ is independently hydrogen or C₁₋₆ alkyl, or two R¹⁰ together with the atom(s) attached thereto form a 4- to 6-membered ring; and
R¹¹ is hydrogen or R³.

The compound may be a compound described in International Patent Publication No. WO 2013/013240. The compound may be a compound of formula II:

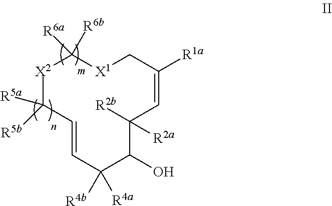

or a pharmaceutically acceptable salt thereof;
wherein n and m are each independently 0, 1, 2, 3 or 4;
X¹ is —O—, —NR^{7a}—, —CR^{9a}R^{9b}—, —C(O)—NR^{7a}—, —NR^{7a}—C(O)—, —NR^{8a}—S(O)₂— or —S(O)₂—NR^{8a}—;
X² is —NR^{8a}—, —CR^{9a}R^{9b}—, —S—, —O—, —S(O)—, —S(O)₂—, —C(O)—NR^{8a}—, —NR^{8a}—C(O)—, —NR^{8a}—S(O)₂— or —S(O)₂—NR—;
R^{1a} is halo, C₁₋₆ alkyl or C₁₋₆ alkoxy;
each R^{2a} and R^{2b} is independently hydrogen or C₁₋₆ alkyl;
each R^{4a} and R^{4b} is independently hydrogen, hydroxyl, C₁₋₆ alkyl or C₁₋₆ alkoxy;
each R^{5a} and R^{5b} is independently hydrogen, halo, amino, amido, C₁₋₆ alkyl or C₁₋₆ alkoxy;
each R^{6a} and R^{6b} is independently hydrogen, halo, amino, amido, C₁₋₆ alkyl or C₁₋₆ alkoxy;
R^{7a} is hydrogen, C₁₋₆ alkyl, acyl, aryl or aralkyl;
R^{8a} is hydrogen, C₁₋₆ alkyl, acyl, aryl or aralkyl; and
each R^{9a} and R^{9b} is independently hydrogen, C₁₋₆ alkyl, C₁₋₆ alkoxy, halo, amino, amido, aryl or heteroaryl.

The compound may be a compound described in U.S. Patent Publication No. 2014/0024705. The compound may be a compound of formula IV:

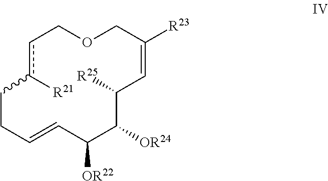

or a pharmaceutically acceptable salt thereof;

wherein:

[symbol] is a single or double bond;

R²¹ is hydrogen or optionally substituted C₁₋₆ alkyl;

R²² is an oxygen protecting group, hydrogen, or optionally substituted C₁₋₆ alkyl;

R²³ and R²⁵ are each independently optionally substituted C₁₋₆ alkyl; and

R²⁴ is hydrogen or -T-Y;

-T- is an optionally substituted C₁₋₈ bivalent saturated or unsaturated, straight or branched, hydrocarbon chain, wherein one or more methylene units are optionally and independently replaced by —NR²⁶—, —N(R²⁶)C(O)—, —C(O)N(R²⁶)—, —N(R²⁶)SO₂—, —SO₂N(R²⁶)—, —O—, —C(O)—, —OC(O)—, —OC(O)O—, —C(O)O—, —OC(O)N(R²⁶)—, —S—, —SO— or —SO₂—;

each R²⁶ is independently hydrogen, or an optionally substituted group selected from the group consisting of C₁₋₂₀ alkyl, C₁₋₂₀ heteroalkyl, 6- to 10-membered aryl, 5- to 12-membered heteroaryl, 3- to 14-membered cycloalkyl, 3- to 12-membered heterocyclyl; and

—Y is hydrogen or acyl.

The compound may be a compound described in U.S. Patent Publication No. 2014/0080843. The compound may be a compound of formula V:

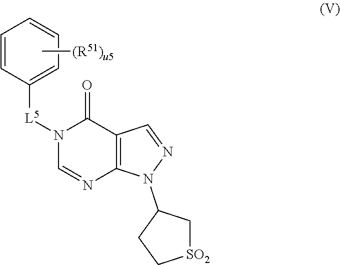

wherein
L⁵ is selected from the group consisting of —(C(R⁵⁸)₂)_{q}—, —(C(R⁵⁸)₂)_{q}—C(O)—(C(R⁵⁸)₂)ᵣ—, —(C(R⁵⁸)₂)_{q}—C(O)N(R⁵⁸)—(C(R⁵⁸)₂)ᵣ—, —(C(R⁵⁸)₂)_{q}—N(R⁵⁸)C(O)—(C(R⁵⁸)₂)ᵣ—, —(C(R⁵⁸)₂)_{q}—N(R⁵⁸)S(O)₂—(C(R⁵⁸)₂)ᵣ, —(CH₂)_{q}—S(O)₂N(R⁵⁸)—(CH₂)ᵣ—, —S—, —O— and —NR⁵⁸—;
each R⁵¹ is independently selected from the group consisting of halo and C₁₋₆ alkyl optionally substituted with 1-3 halo; or two adjacent R⁵¹ on a phenyl ring form a 5- or 6-membered cycloalkyl or heterocyclyl fused with the phenyl ring;
each R⁵⁸ is independently hydrogen or C₁₋₆ alkyl;
q is 0 or 1;
r is 0 or 1; and
u5 is 1, 2 or 3;
or a pharmaceutically acceptable salt thereof.

In some embodiments, the compound may be a compound described in U.S. Patent Publication No. 2014/0080843. In some embodiments, the compound is a compound of Formula I-a or Formula I-b

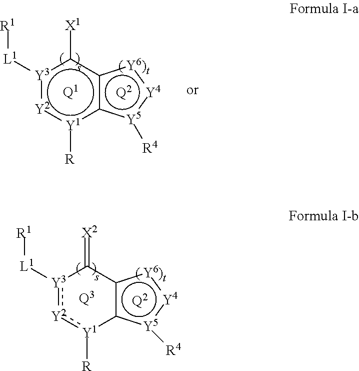

Q¹ and Q² of Formula I-a or Formula I-b are independently phenyl, 5-membered heteroaryl or 6-membered heteroaryl and are fused together in Formula I-a;
Q³ of Formula I-b is 6-membered unsaturated ring wherein (1) the bond between Y¹ and Y² is a double bond, and the bond between Y and Y² is a single bond, or (2) the bond between Y¹ and Y² is a single bond, and the bond between Y³ and Y² is a double bond, and wherein Q³ is fused with Q² in Formula I-b;

### s of Formula I-a or Formula I-b is 0 or 1;

### t of Formula I-a or Formula I-b is 1 or 2:

Y¹, Y³ and Y⁴ of Formula I-a or Formula I-b are independently C or N; Y², Y⁴ and Y⁶ of Formula I-a or Formula I-b are independently CH, CR³ or N; provided that no more than four of Y¹, Y², Y³, Y⁴, Y⁵ and Y⁶ of Formula I-a or of Formula I-b are N;
R¹ of Formula I-a or Formula I-b is phenyl, 5-membered heteroaryl or 6-membered heteroaryl, wherein the phenyl, 5-membered heteroaryl or 6-membered heteroaryl is optionally substituted with 1 to 3 R⁶ of Formula I-a or Formula I-b;
one of R and R⁴ of Formula I-a or Formula I-b is absent or is hydrogen, halo or lower alkyl (preferably methyl or ethyl), and the other of R and R⁴ is L²-R⁵ or L³-R³; or R is absent and R⁴ is —(CH₂)ⱼ—R^{11a}; R^{11a} of Formula I-a or Formula I-b is selected from the group consisting of —OH, —OR⁷, —SH, —SR⁷, —NR¹⁰R¹⁰, cyano, nitro, —COH, —COR⁷, —CO₂H, —CO₂R⁷, —CONR¹⁰R¹⁰, —OCOR⁷, —OCO₂R⁷, —OCONR¹⁰R¹⁰, —NR¹⁰COR¹⁰, —NR¹⁰CO₂R¹⁰, —SOR⁷, —SO₂R⁷, —SO₂NR¹⁰R¹⁰, and —NR¹⁰SO₂R⁷,
X¹ of Formula I-a or Formula I-b is selected from the group consisting of OR⁸, NHR⁸, and SR⁸;
X² of Formula I-a or Formula I-b is selected from the group consisting of O, NR⁸, and S;
L¹ of Formula I-a or Formula I-b is selected from the group consisting —(C(R⁸)₂)ⱼ—, —(C(R⁸)₂)_{q}—C(O)—(C(R⁸)₂)ᵣ—, —(C(R⁸)₂)_{q}—C(O)N(R⁸)—(C(R⁸)₂)ᵣ—, —(C(R⁸)₂)_{q}—N(R⁸)C(O)—(C(R⁸)₂)ᵣ—, —(C(R⁸)₂)_{q}—N(R⁸)S(O)₂—(C(R⁸)₂)ᵣ—, —(CH₂)_{q}—S(O)₂N(R⁸)—(CH₂)ᵣ—, —S—, —O— and —NR⁸—;

### j of Formula I-a or Formula I-b is 1, 2 or 3;

### q of Formula I-a or Formula I-b is 0 or 1;

### r of Formula I-a or Formula I-b is 0 or 1;

L² of Formula I-a or Formula I-b is selected from the group consisting a covalent bond, —C(O)N(R⁸)—, —N(R⁸)C(O)—, —N(R⁸)S(O)₂—, and —S(O)₂N(R⁸)—;
L³ of Formula I-a or Formula I-b is ═NC(O)—, or ═NS(O)₂—;
each R³ of Formula I-a or Formula I-b is independently selected from the group consisting of lower alkyl (preferably methyl or ethyl) and halo;
R⁵ of Formula I-a or Formula I-b is phenyl, 5-membered heteroaryl, 6-membered heteroaryl, 5-membered heterocycloalkyl or 6-membered heterocycloalkyl; wherein the phenyl, 5-membered heteroaryl or 6-membered heteroaryl is optionally substituted with 1 to 4 R², wherein each R² of Formula I-a or Formula I-b is independently selected from the group consisting of lower alkyl, lower haloalkyl, —OH, —OR⁷, —SH, —SR⁷, —NR¹⁰R¹⁰, halo, cyano, nitro, —COH, —COR⁷, —CO₂H, —CO₂R⁷, —CONR¹⁰R¹⁰, —OCOR⁷, —OCO₂R⁷, —OCONR¹⁰R¹⁰, —NR¹⁰COR¹⁰, —NR¹⁰CO₂R¹⁰, —S(O)R⁷, —SO₂R⁷, —SO₂NR¹⁰R¹⁰, and —NR¹⁰SO₂R⁷;
each R⁶ of Formula I-a or Formula I-b is independently selected from the group consisting of halo and lower alkyl (preferably methyl or ethyl) optionally substituted with 1-3 halo; or two adjacent R⁶ on a phenyl ring form a 5- or 6-membered cycloalkyl or heterocycloalkyl fused with the phenyl ring;
R⁷ of Formula I-a or Formula I-b is lower alkyl (preferably methyl or ethyl);
R⁸ of Formula I-a or Formula I-b is hydrogen or lower alkyl (preferably methyl or ethyl); and each R¹⁰ of Formula I-a or Formula I-b is independently hydrogen or lower alkyl (preferably methyl or ethyl), or two R¹⁰ together with the atom(s) attached thereto form a 4- to 6-membered ring.

In some embodiments, provided is a compound according to Formula VII:

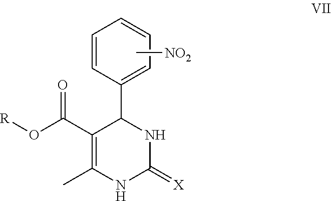

wherein: the nitro group is ortho or meta; R of Formula VII is methyl or ethyl; and X of Formula VII is O or S; or a pharmaceutically acceptable salt thereof.

In some embodiments, provided is a compound according to Formula VIII:

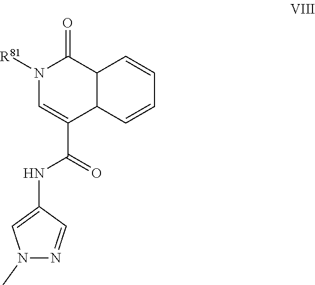

wherein: R⁸¹ is

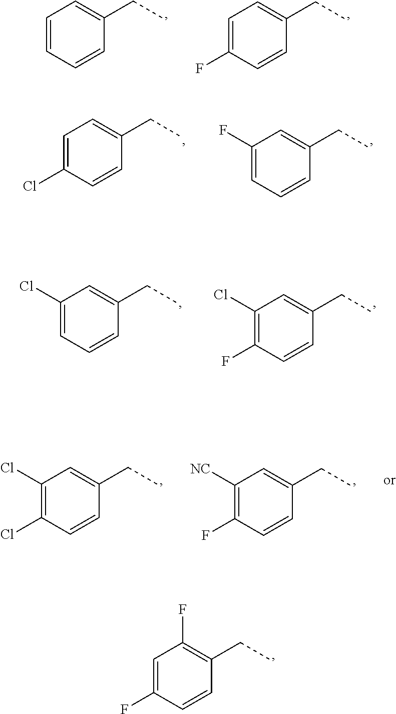

wherein [symbol] represents a point of attachment; or a pharmaceutically acceptable salt thereof.

In some embodiments, provided is a compound according to Formula IX:

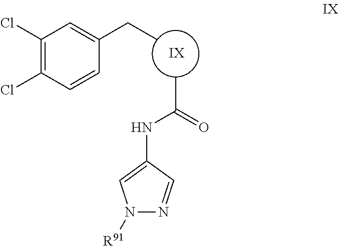

wherein R⁹¹ is methyl or 3-pyrrolidine, and Ring IX is

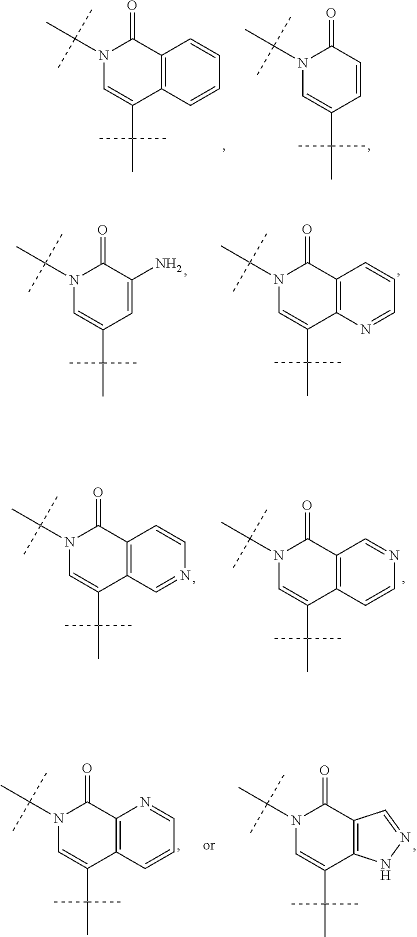

wherein

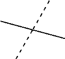

indicates a point of attachment; or a pharmaceutically acceptable salt thereof.

In some embodiments, provided is a compound according to Formula X:

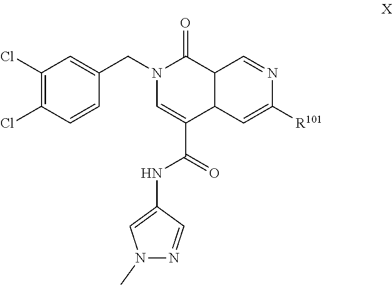

wherein R¹⁰¹ is H,

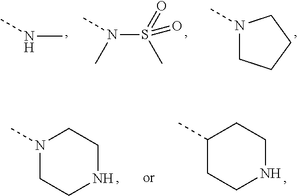

wherein [symbol] represents a point of attachment; or a pharmaceutically acceptable salt thereof.

In some embodiments, provided is a compound according to Formula XI:

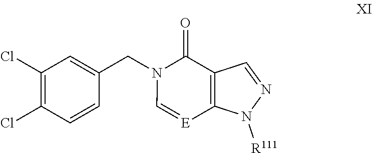

wherein E is N, or E is C substituted by

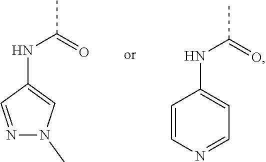

### and R¹¹¹ is H,

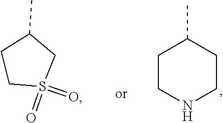

wherein [symbol] represents a point of attachment; or a pharmaceutically acceptable salt thereof.

In some embodiments, the compound is selected from:

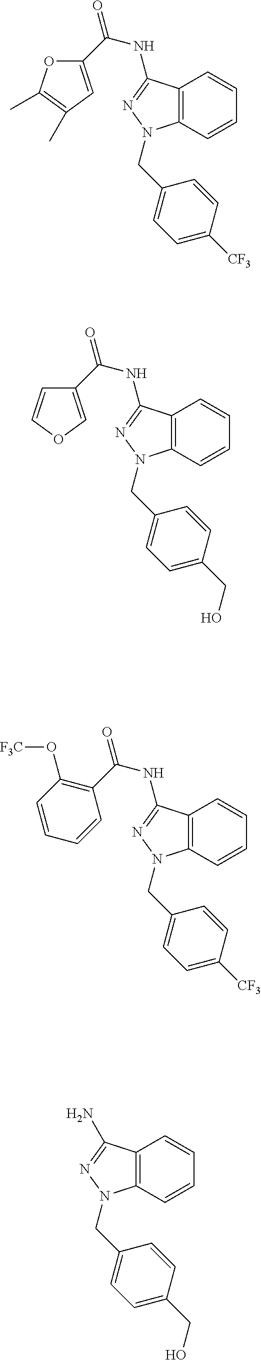 [SMILES: Cc1cc(C(=O)Nc2nn(Cc3ccc(C(F)(F)F)cc3)c3ccccc23)oc1C ; O=C(Nc1nn(Cc2ccc(CO)cc2)c2ccccc12)c1ccoc1 ; O=C(Nc1nn(Cc2ccc(C(F)(F)F)cc2)c2ccccc12)c1ccccc1OC(F)(F)F ; Nc1nn(Cc2ccc(CO)cc2)c2ccccc12]

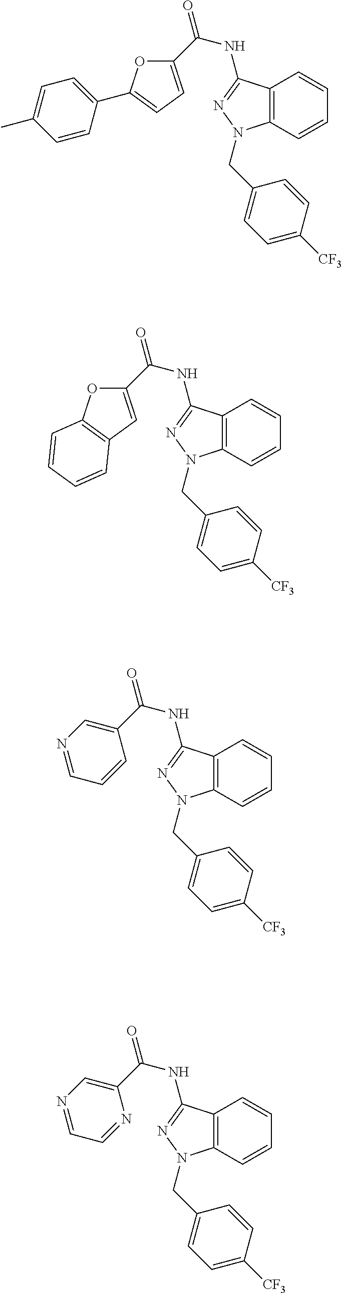 [SMILES: Cc1ccc(-c2ccc(C(=O)Nc3nn(Cc4ccc(C(F)(F)F)cc4)c4ccccc34)o2)cc1 ; O=C(Nc1nn(Cc2ccc(C(F)(F)F)cc2)c2ccccc12)c1cc2ccccc2o1 ; O=C(Nc1nn(Cc2ccc(C(F)(F)F)cc2)c2ccccc12)c1cccnc1 ; O=C(Nc1nn(Cc2ccc(C(F)(F)F)cc2)c2ccccc12)c1cnccn1]

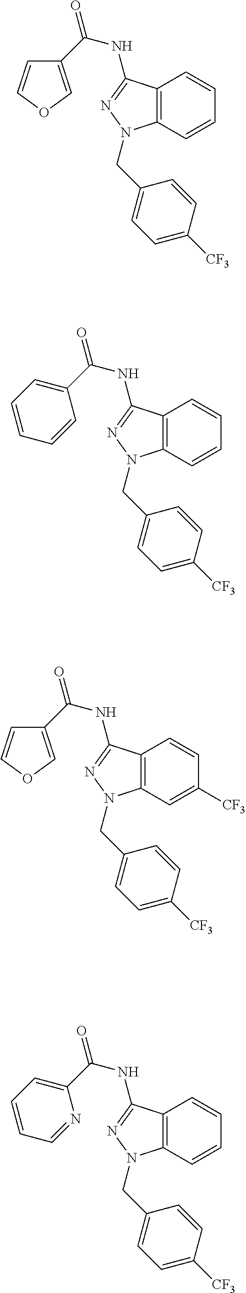 [SMILES: O=C(Nc1nn(Cc2ccc(C(F)(F)F)cc2)c2ccccc12)c1ccoc1 ; O=C(Nc1nn(Cc2ccc(C(F)(F)F)cc2)c2ccccc12)c1ccccc1 ; O=C(Nc1nn(Cc2ccc(C(F)(F)F)cc2)c2cc(C(F)(F)F)ccc12)c1ccoc1 ; O=C(Nc1nn(Cc2ccc(C(F)(F)F)cc2)c2ccccc12)c1ccccn1]

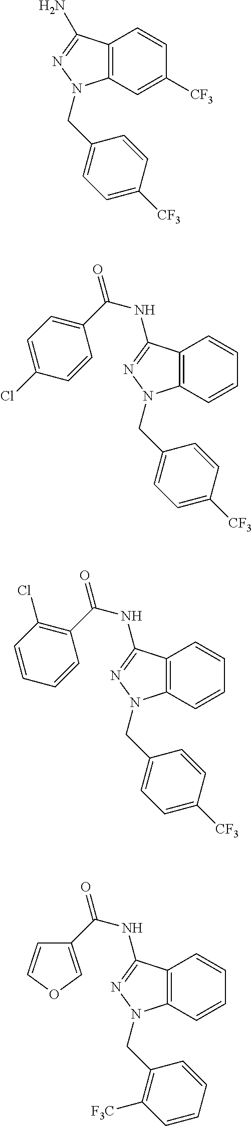 [SMILES: Nc1nn(Cc2ccc(C(F)(F)F)cc2)c2cc(C(F)(F)F)ccc12 ; O=C(Nc1nn(Cc2ccc(C(F)(F)F)cc2)c2ccccc12)c1ccc(Cl)cc1 ; O=C(Nc1nn(Cc2ccc(C(F)(F)F)cc2)c2ccccc12)c1ccccc1Cl ; O=C(Nc1nn(Cc2ccccc2C(F)(F)F)c2ccccc12)c1ccoc1]

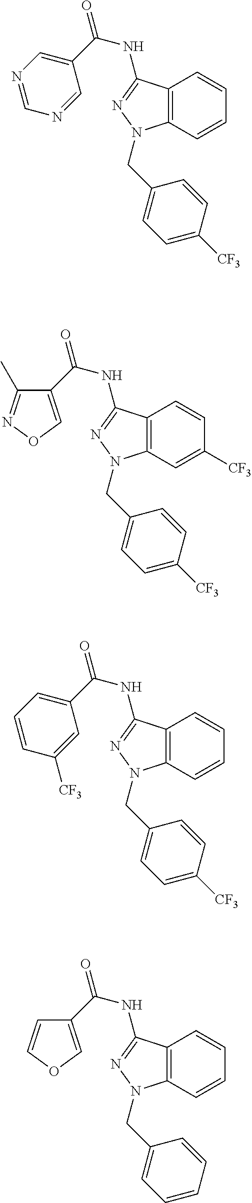 [SMILES: O=C(Nc1nn(Cc2ccc(C(F)(F)F)cc2)c2ccccc12)c1cncnc1 ; Cc1nocc1C(=O)Nc1nn(Cc2ccc(C(F)(F)F)cc2)c2cc(C(F)(F)F)ccc12 ; O=C(Nc1nn(Cc2ccc(C(F)(F)F)cc2)c2ccccc12)c1cccc(C(F)(F)F)c1 ; O=C(Nc1nn(Cc2ccccc2)c2ccccc12)c1ccoc1]

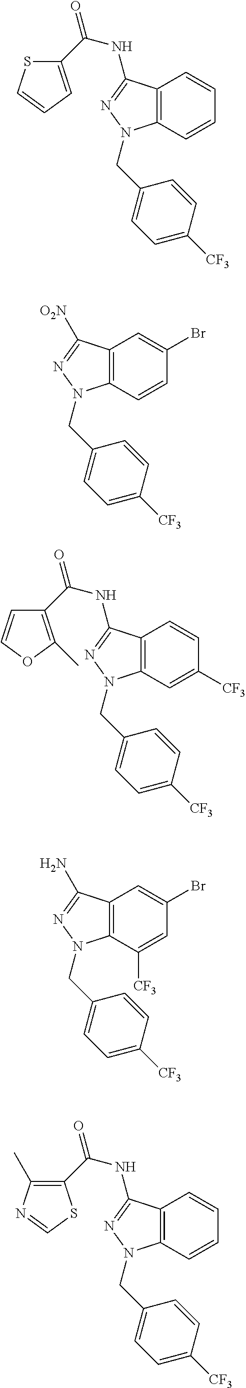 [SMILES: O=C(Nc1nn(Cc2ccc(C(F)(F)F)cc2)c2ccccc12)c1cccs1 ; O=[N+]([O-])c1nn(Cc2ccc(C(F)(F)F)cc2)c2ccc(Br)cc12 ; Cc1occc1C(=O)Nc1nn(Cc2ccc(C(F)(F)F)cc2)c2cc(C(F)(F)F)ccc12 ; Nc1nn(Cc2ccc(C(F)(F)F)cc2)c2c(C(F)(F)F)cc(Br)cc12 ; Cc1ncsc1C(=O)Nc1nn(Cc2ccc(C(F)(F)F)cc2)c2ccccc12]

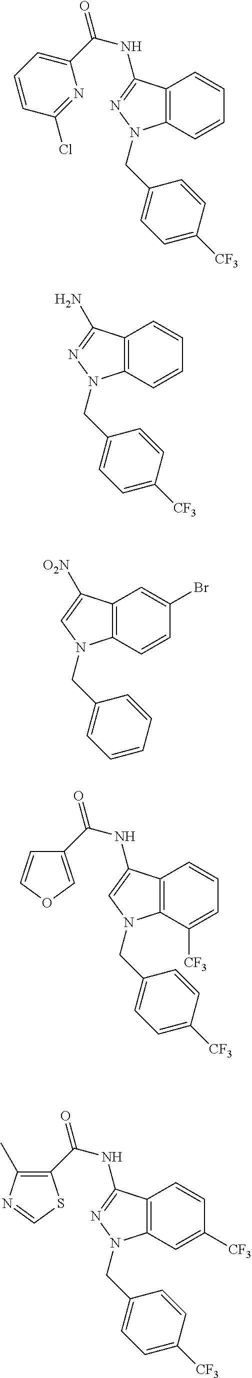 [SMILES: O=C(Nc1nn(Cc2ccc(C(F)(F)F)cc2)c2ccccc12)c1cccc(Cl)n1 ; Nc1nn(Cc2ccc(C(F)(F)F)cc2)c2ccccc12 ; O=[N+]([O-])c1cn(Cc2ccccc2)c2ccc(Br)cc12 ; O=C(Nc1cn(Cc2ccc(C(F)(F)F)cc2)c2c(C(F)(F)F)cccc12)c1ccoc1 ; Cc1ncsc1C(=O)Nc1nn(Cc2ccc(C(F)(F)F)cc2)c2cc(C(F)(F)F)ccc12]

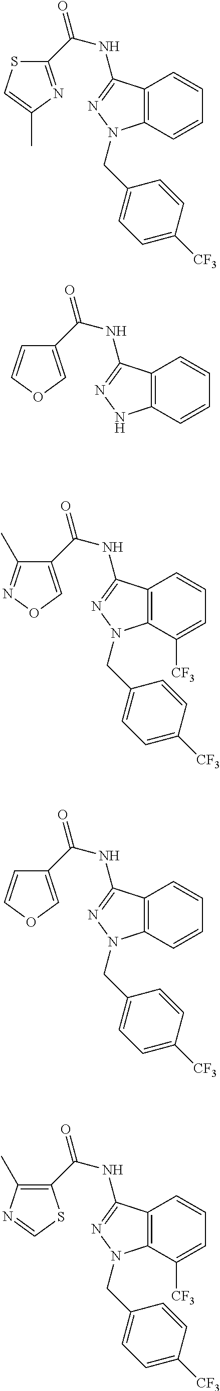 [SMILES: Cc1csc(C(=O)Nc2nn(Cc3ccc(C(F)(F)F)cc3)c3ccccc23)n1 ; O=C(Nc1n[nH]c2ccccc12)c1ccoc1 ; Cc1nocc1C(=O)Nc1nn(Cc2ccc(C(F)(F)F)cc2)c2c(C(F)(F)F)cccc12 ; O=C(Nc1nn(Cc2ccc(C(F)(F)F)cc2)c2ccccc12)c1ccoc1 ; Cc1ncsc1C(=O)Nc1nn(Cc2ccc(C(F)(F)F)cc2)c2c(C(F)(F)F)cccc12]

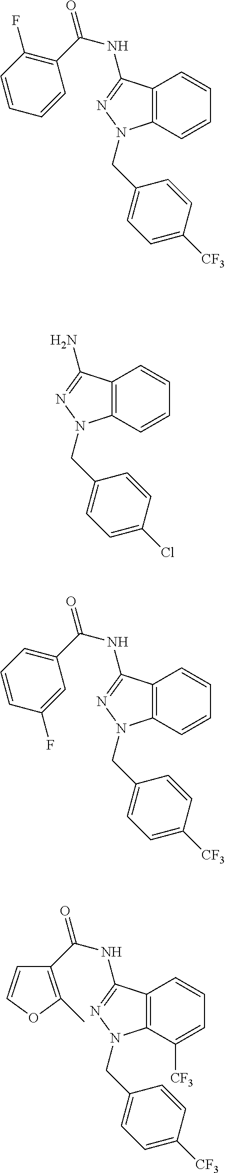 [SMILES: O=C(Nc1nn(Cc2ccc(C(F)(F)F)cc2)c2ccccc12)c1ccccc1F ; Nc1nn(Cc2ccc(Cl)cc2)c2ccccc12 ; O=C(Nc1nn(Cc2ccc(C(F)(F)F)cc2)c2ccccc12)c1cccc(F)c1 ; Cc1occc1C(=O)Nc1nn(Cc2ccc(C(F)(F)F)cc2)c2c(C(F)(F)F)cccc12]

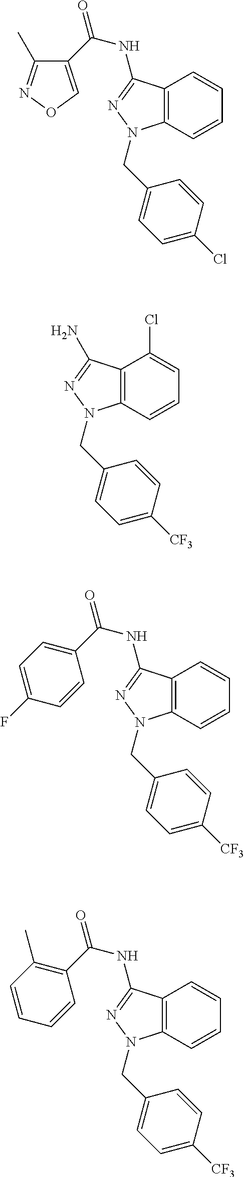 [SMILES: Cc1nocc1C(=O)Nc1nn(Cc2ccc(Cl)cc2)c2ccccc12 ; Nc1nn(Cc2ccc(C(F)(F)F)cc2)c2cccc(Cl)c12 ; O=C(Nc1nn(Cc2ccc(C(F)(F)F)cc2)c2ccccc12)c1ccc(F)cc1 ; Cc1ccccc1C(=O)Nc1nn(Cc2ccc(C(F)(F)F)cc2)c2ccccc12]

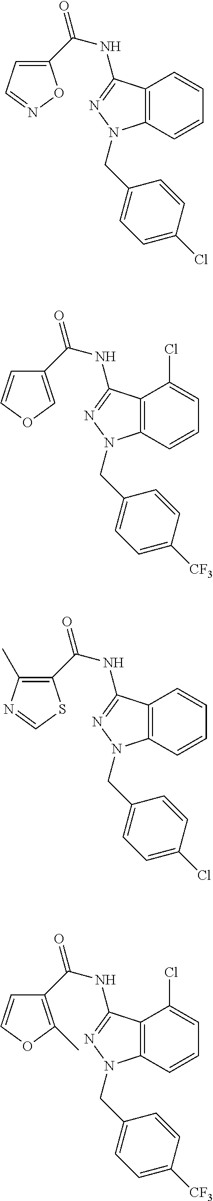 [SMILES: O=C(Nc1nn(Cc2ccc(Cl)cc2)c2ccccc12)c1ccno1 ; O=C(Nc1nn(Cc2ccc(C(F)(F)F)cc2)c2cccc(Cl)c12)c1ccoc1 ; Cc1ncsc1C(=O)Nc1nn(Cc2ccc(Cl)cc2)c2ccccc12 ; Cc1occc1C(=O)Nc1nn(Cc2ccc(C(F)(F)F)cc2)c2cccc(Cl)c12]

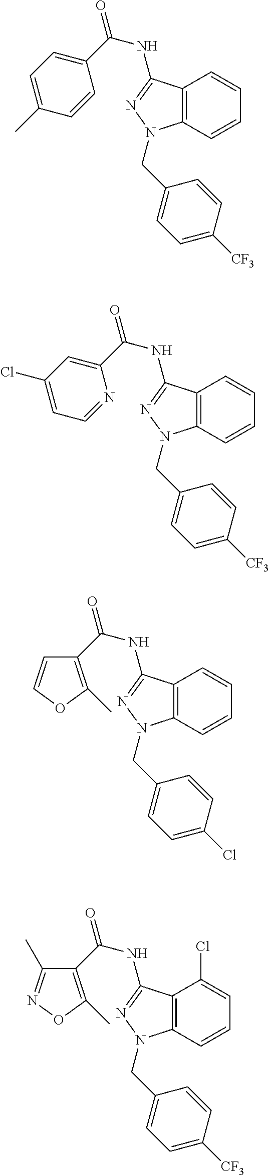 [SMILES: Cc1ccc(C(=O)Nc2nn(Cc3ccc(C(F)(F)F)cc3)c3ccccc23)cc1 ; O=C(Nc1nn(Cc2ccc(C(F)(F)F)cc2)c2ccccc12)c1cc(Cl)ccn1 ; Cc1occc1C(=O)Nc1nn(Cc2ccc(Cl)cc2)c2ccccc12 ; Cc1noc(C)c1C(=O)Nc1nn(Cc2ccc(C(F)(F)F)cc2)c2cccc(Cl)c12]

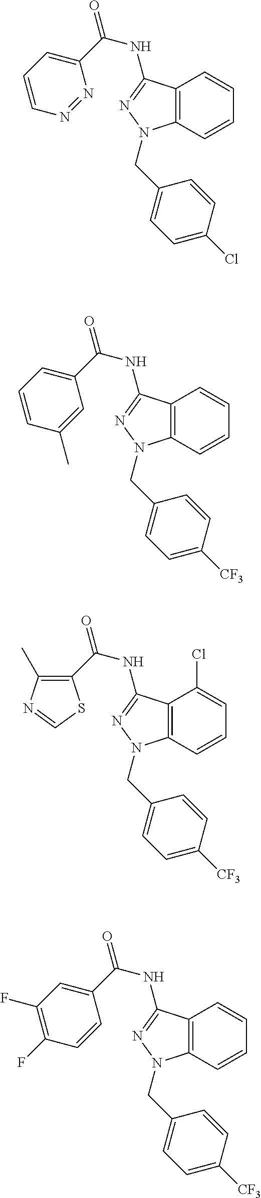 [SMILES: O=C(Nc1nn(Cc2ccc(Cl)cc2)c2ccccc12)c1cccnn1 ; Cc1cccc(C(=O)Nc2nn(Cc3ccc(C(F)(F)F)cc3)c3ccccc23)c1 ; Cc1ncsc1C(=O)Nc1nn(Cc2ccc(C(F)(F)F)cc2)c2cccc(Cl)c12 ; O=C(Nc1nn(Cc2ccc(C(F)(F)F)cc2)c2ccccc12)c1ccc(F)c(F)c1]

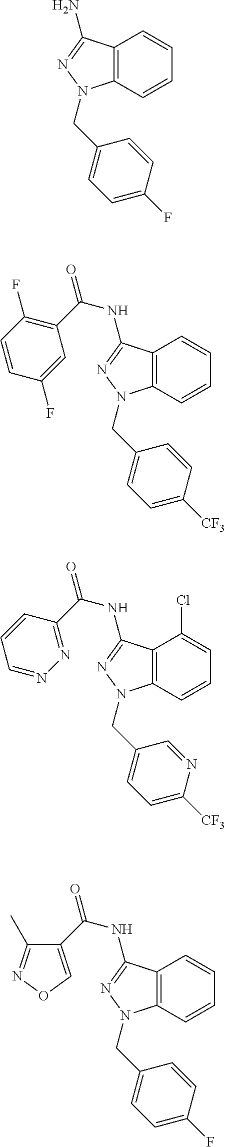 [SMILES: Nc1nn(Cc2ccc(F)cc2)c2ccccc12 ; O=C(Nc1nn(Cc2ccc(C(F)(F)F)cc2)c2ccccc12)c1cc(F)ccc1F ; O=C(Nc1nn(Cc2ccc(C(F)(F)F)nc2)c2cccc(Cl)c12)c1cccnn1 ; Cc1nocc1C(=O)Nc1nn(Cc2ccc(F)cc2)c2ccccc12]

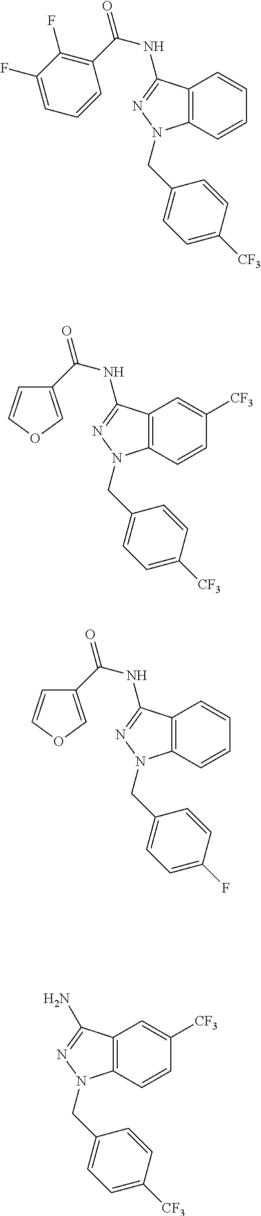 [SMILES: O=C(Nc1nn(Cc2ccc(C(F)(F)F)cc2)c2ccccc12)c1cccc(F)c1F ; O=C(Nc1nn(Cc2ccc(C(F)(F)F)cc2)c2ccc(C(F)(F)F)cc12)c1ccoc1 ; O=C(Nc1nn(Cc2ccc(F)cc2)c2ccccc12)c1ccoc1 ; Nc1nn(Cc2ccc(C(F)(F)F)cc2)c2ccc(C(F)(F)F)cc12]

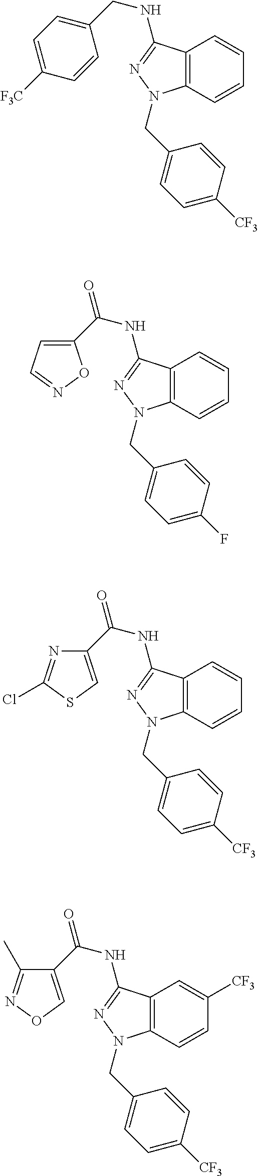 [SMILES: FC(F)(F)c1ccc(CNc2nn(Cc3ccc(C(F)(F)F)cc3)c3ccccc23)cc1 ; O=C(Nc1nn(Cc2ccc(F)cc2)c2ccccc12)c1ccno1 ; O=C(Nc1nn(Cc2ccc(C(F)(F)F)cc2)c2ccccc12)c1csc(Cl)n1 ; Cc1nocc1C(=O)Nc1nn(Cc2ccc(C(F)(F)F)cc2)c2ccc(C(F)(F)F)cc12]

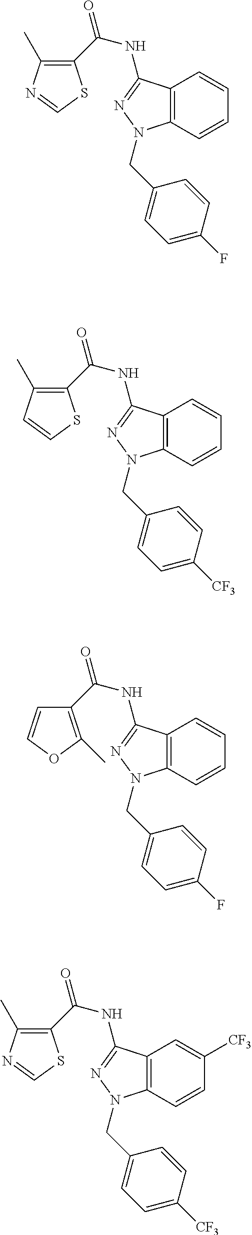 [SMILES: Cc1ncsc1C(=O)Nc1nn(Cc2ccc(F)cc2)c2ccccc12 ; Cc1ccsc1C(=O)Nc1nn(Cc2ccc(C(F)(F)F)cc2)c2ccccc12 ; Cc1occc1C(=O)Nc1nn(Cc2ccc(F)cc2)c2ccccc12 ; Cc1ncsc1C(=O)Nc1nn(Cc2ccc(C(F)(F)F)cc2)c2ccc(C(F)(F)F)cc12]

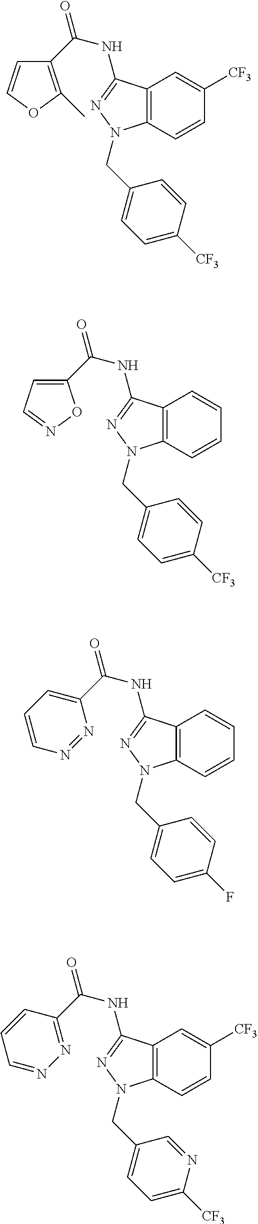 [SMILES: Cc1occc1C(=O)Nc1nn(Cc2ccc(C(F)(F)F)cc2)c2ccc(C(F)(F)F)cc12 ; O=C(Nc1nn(Cc2ccc(C(F)(F)F)cc2)c2ccccc12)c1ccno1 ; O=C(Nc1nn(Cc2ccc(F)cc2)c2ccccc12)c1cccnn1 ; O=C(Nc1nn(Cc2ccc(C(F)(F)F)nc2)c2ccc(C(F)(F)F)cc12)c1cccnn1]

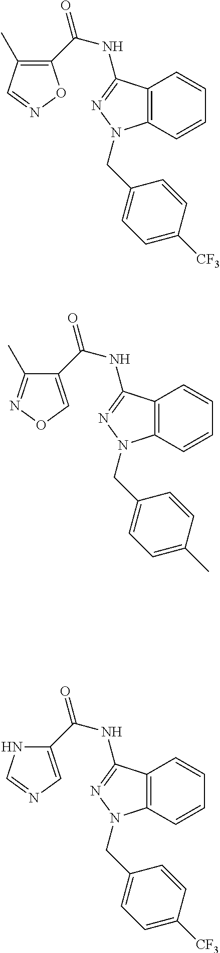 [SMILES: Cc1cnoc1C(=O)Nc1nn(Cc2ccc(C(F)(F)F)cc2)c2ccccc12 ; Cc1ccc(Cn2nc(NC(=O)c3conc3C)c3ccccc32)cc1 ; O=C(Nc1nn(Cc2ccc(C(F)(F)F)cc2)c2ccccc12)c1cnc[nH]1]

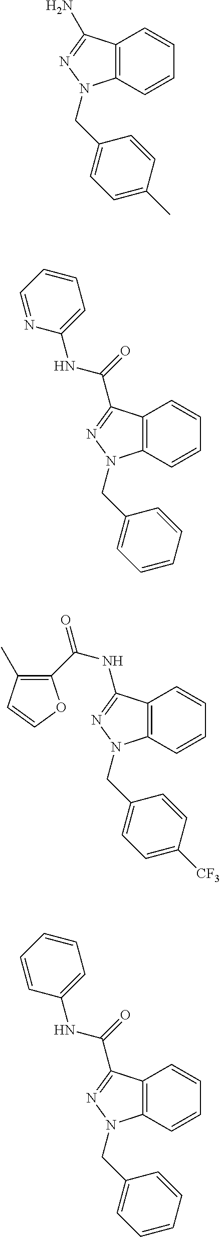 [SMILES: Cc1ccc(Cn2nc(N)c3ccccc32)cc1 ; O=C(Nc1ccccn1)c1nn(Cc2ccccc2)c2ccccc12 ; Cc1ccoc1C(=O)Nc1nn(Cc2ccc(C(F)(F)F)cc2)c2ccccc12 ; O=C(Nc1ccccc1)c1nn(Cc2ccccc2)c2ccccc12]

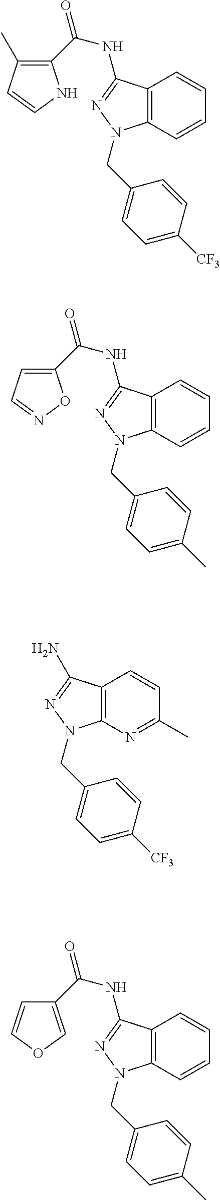 [SMILES: Cc1cc[nH]c1C(=O)Nc1nn(Cc2ccc(C(F)(F)F)cc2)c2ccccc12 ; Cc1ccc(Cn2nc(NC(=O)c3ccno3)c3ccccc32)cc1 ; Cc1ccc2c(N)nn(Cc3ccc(C(F)(F)F)cc3)c2n1 ; Cc1ccc(Cn2nc(NC(=O)c3ccoc3)c3ccccc32)cc1]

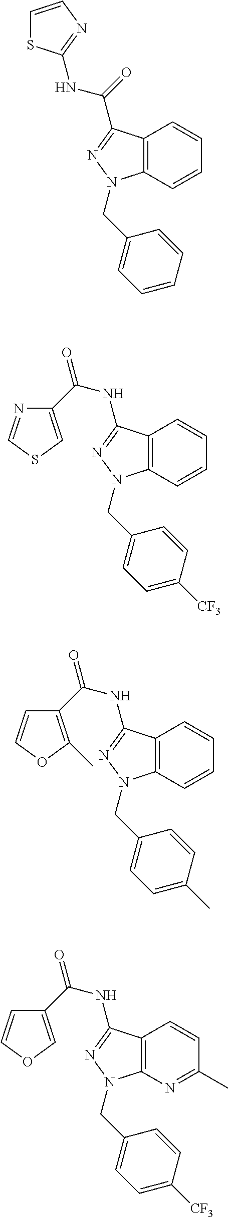 [SMILES: O=C(Nc1nccs1)c1nn(Cc2ccccc2)c2ccccc12 ; O=C(Nc1nn(Cc2ccc(C(F)(F)F)cc2)c2ccccc12)c1cscn1 ; Cc1ccc(Cn2nc(NC(=O)c3ccoc3C)c3ccccc32)cc1 ; Cc1ccc2c(NC(=O)c3ccoc3)nn(Cc3ccc(C(F)(F)F)cc3)c2n1]

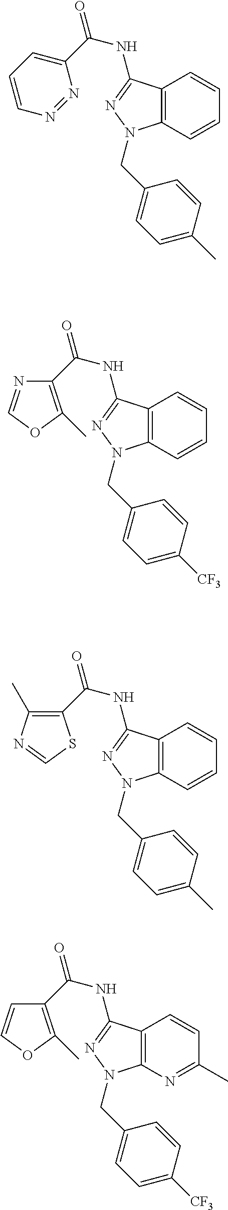 [SMILES: Cc1ccc(Cn2nc(NC(=O)c3cccnn3)c3ccccc32)cc1 ; Cc1ocnc1C(=O)Nc1nn(Cc2ccc(C(F)(F)F)cc2)c2ccccc12 ; Cc1ccc(Cn2nc(NC(=O)c3scnc3C)c3ccccc32)cc1 ; Cc1ccc2c(NC(=O)c3ccoc3C)nn(Cc3ccc(C(F)(F)F)cc3)c2n1]

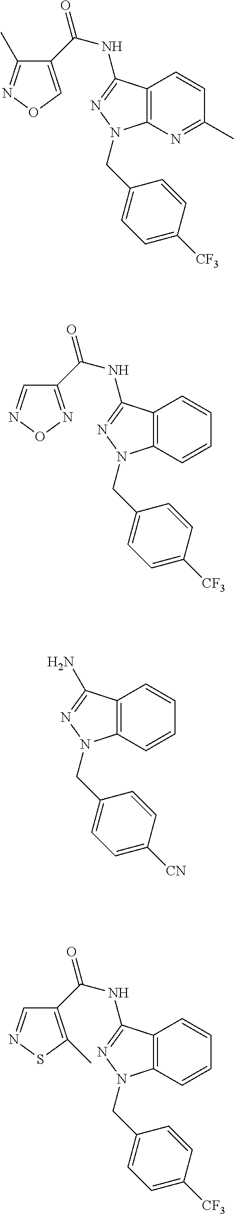

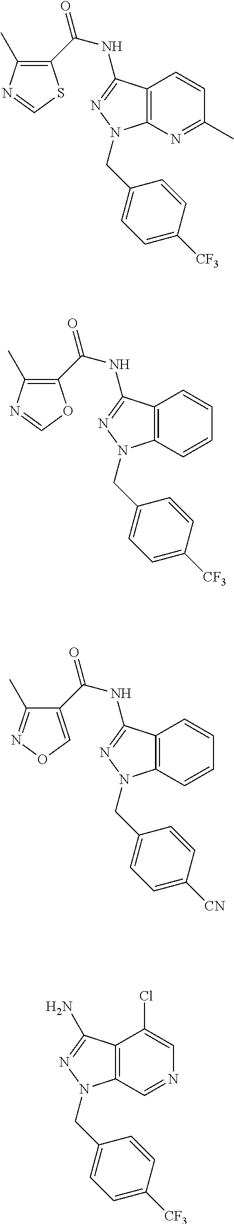

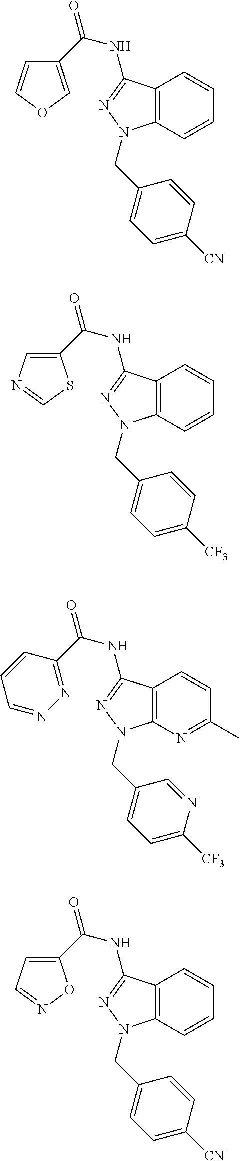 [SMILES: N#Cc1ccc(Cn2nc(NC(=O)c3ccoc3)c3ccccc32)cc1 ; O=C(Nc1nn(Cc2ccc(C(F)(F)F)cc2)c2ccccc12)c1cncs1 ; Cc1ccc2c(NC(=O)c3cccnn3)nn(Cc3ccc(C(F)(F)F)nc3)c2n1 ; N#Cc1ccc(Cn2nc(NC(=O)c3ccno3)c3ccccc32)cc1]

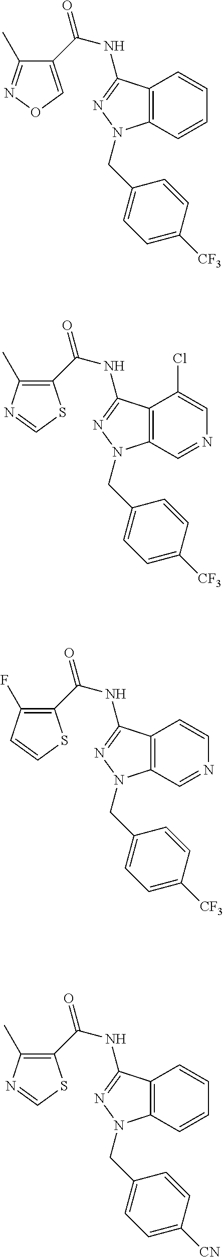 [SMILES: Cc1nocc1C(=O)Nc1nn(Cc2ccc(C(F)(F)F)cc2)c2ccccc12 ; Cc1ncsc1C(=O)Nc1nn(Cc2ccc(C(F)(F)F)cc2)c2cncc(Cl)c12 ; O=C(Nc1nn(Cc2ccc(C(F)(F)F)cc2)c2cnccc12)c1sccc1F ; Cc1ncsc1C(=O)Nc1nn(Cc2ccc(C#N)cc2)c2ccccc12]

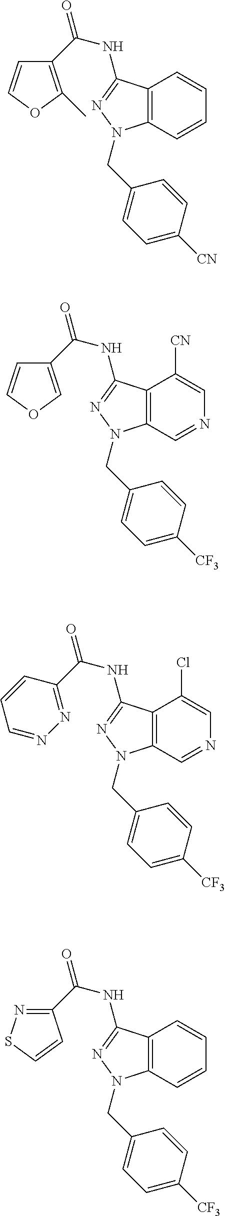 [SMILES: Cc1occc1C(=O)Nc1nn(Cc2ccc(C#N)cc2)c2ccccc12 ; N#Cc1cncc2c1c(NC(=O)c1ccoc1)nn2Cc1ccc(C(F)(F)F)cc1 ; O=C(Nc1nn(Cc2ccc(C(F)(F)F)cc2)c2cncc(Cl)c12)c1cccnn1 ; O=C(Nc1nn(Cc2ccc(C(F)(F)F)cc2)c2ccccc12)c1ccsn1]

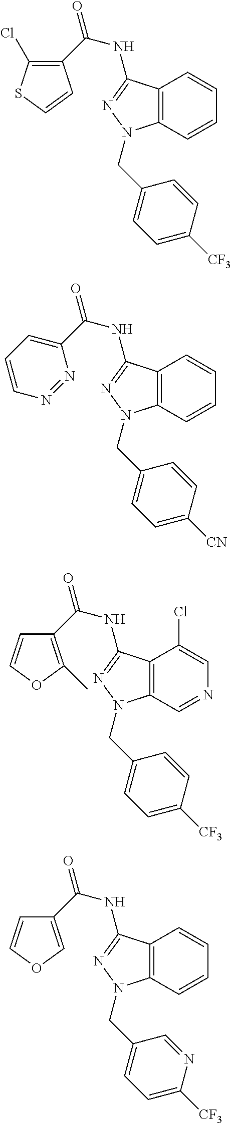 [SMILES: O=C(Nc1nn(Cc2ccc(C(F)(F)F)cc2)c2ccccc12)c1ccsc1Cl ; N#Cc1ccc(Cn2nc(NC(=O)c3cccnn3)c3ccccc32)cc1 ; Cc1occc1C(=O)Nc1nn(Cc2ccc(C(F)(F)F)cc2)c2cncc(Cl)c12 ; O=C(Nc1nn(Cc2ccc(C(F)(F)F)nc2)c2ccccc12)c1ccoc1]

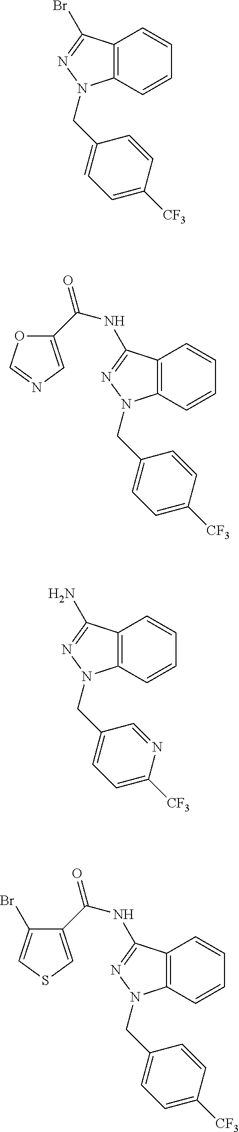 [SMILES: FC(F)(F)c1ccc(Cn2nc(Br)c3ccccc32)cc1 ; O=C(Nc1nn(Cc2ccc(C(F)(F)F)cc2)c2ccccc12)c1cnco1 ; Nc1nn(Cc2ccc(C(F)(F)F)nc2)c2ccccc12 ; O=C(Nc1nn(Cc2ccc(C(F)(F)F)cc2)c2ccccc12)c1cscc1Br]

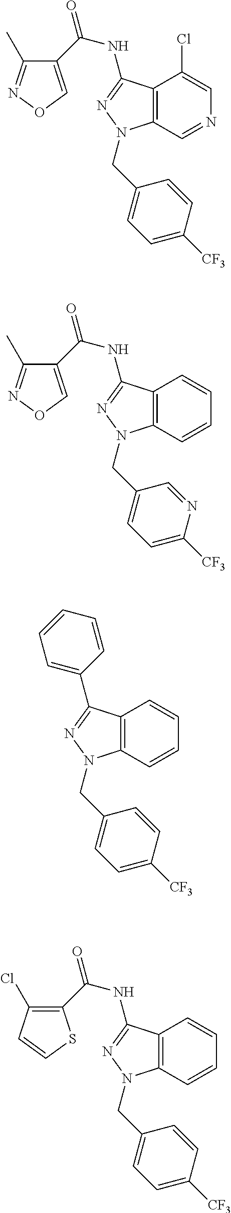 [SMILES: Cc1nocc1C(=O)Nc1nn(Cc2ccc(C(F)(F)F)cc2)c2cncc(Cl)c12 ; Cc1nocc1C(=O)Nc1nn(Cc2ccc(C(F)(F)F)nc2)c2ccccc12 ; FC(F)(F)c1ccc(Cn2nc(-c3ccccc3)c3ccccc32)cc1 ; O=C(Nc1nn(Cc2ccc(C(F)(F)F)cc2)c2ccccc12)c1sccc1Cl]

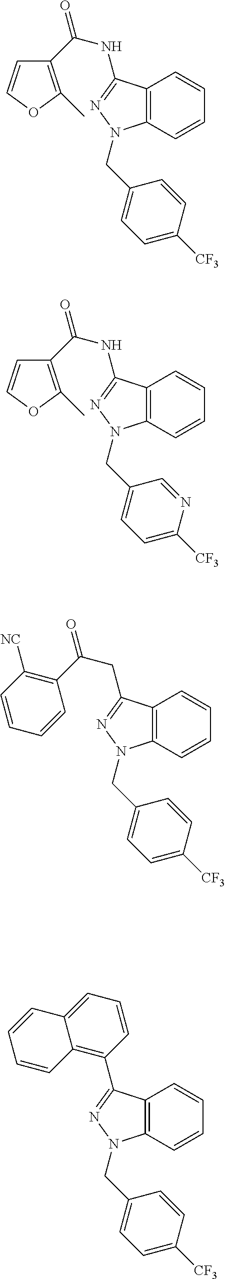 [SMILES: Cc1occc1C(=O)Nc1nn(Cc2ccc(C(F)(F)F)cc2)c2ccccc12 ; Cc1occc1C(=O)Nc1nn(Cc2ccc(C(F)(F)F)nc2)c2ccccc12 ; N#Cc1ccccc1C(=O)Cc1nn(Cc2ccc(C(F)(F)F)cc2)c2ccccc12 ; FC(F)(F)c1ccc(Cn2nc(-c3cccc4ccccc34)c3ccccc32)cc1]

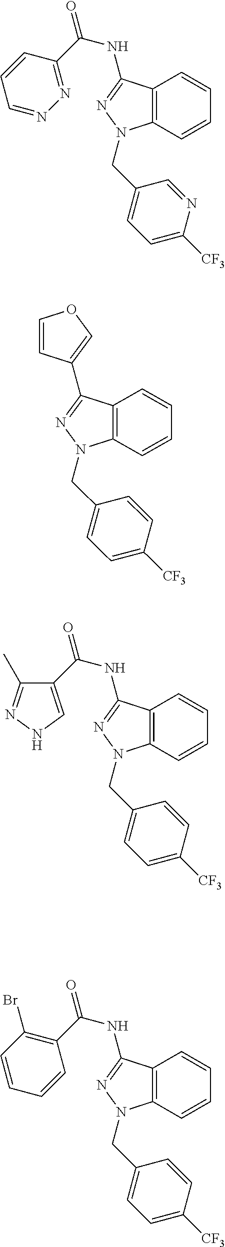 [SMILES: O=C(Nc1nn(Cc2ccc(C(F)(F)F)nc2)c2ccccc12)c1cccnn1 ; FC(F)(F)c1ccc(Cn2nc(-c3ccoc3)c3ccccc32)cc1 ; Cc1n[nH]cc1C(=O)Nc1nn(Cc2ccc(C(F)(F)F)cc2)c2ccccc12 ; O=C(Nc1nn(Cc2ccc(C(F)(F)F)cc2)c2ccccc12)c1ccccc1Br]

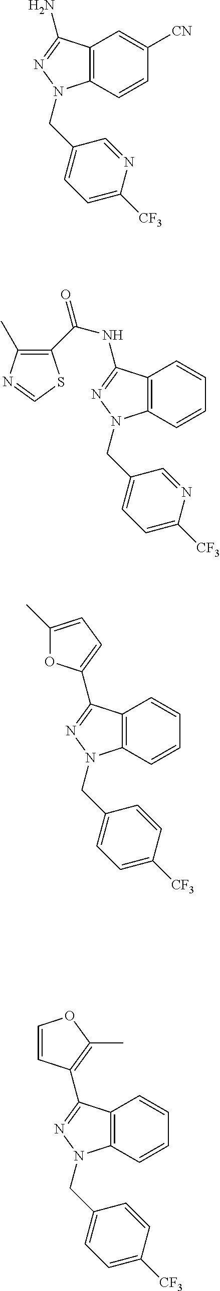 [SMILES: N#Cc1ccc2c(c1)c(N)nn2Cc1ccc(C(F)(F)F)nc1 ; Cc1ncsc1C(=O)Nc1nn(Cc2ccc(C(F)(F)F)nc2)c2ccccc12 ; Cc1ccc(-c2nn(Cc3ccc(C(F)(F)F)cc3)c3ccccc23)o1 ; Cc1occc1-c1nn(Cc2ccc(C(F)(F)F)cc2)c2ccccc12]

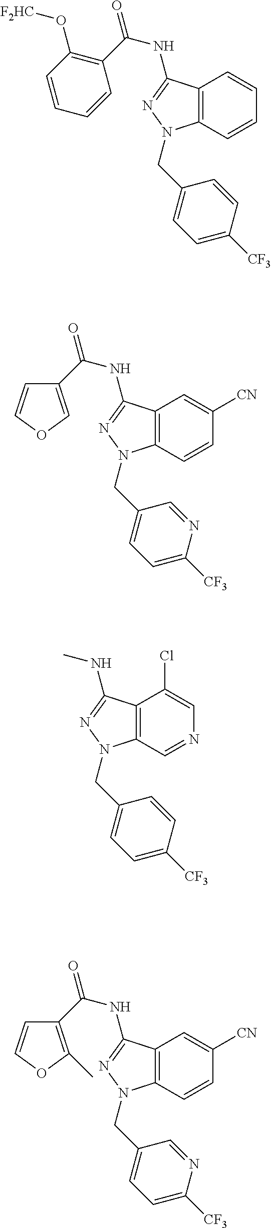 [SMILES: O=C(Nc1nn(Cc2ccc(C(F)(F)F)cc2)c2ccccc12)c1ccccc1OC(F)F ; N#Cc1ccc2c(c1)c(NC(=O)c1ccoc1)nn2Cc1ccc(C(F)(F)F)nc1 ; CNc1nn(Cc2ccc(C(F)(F)F)cc2)c2cncc(Cl)c12 ; Cc1occc1C(=O)Nc1nn(Cc2ccc(C(F)(F)F)nc2)c2ccc(C#N)cc12]

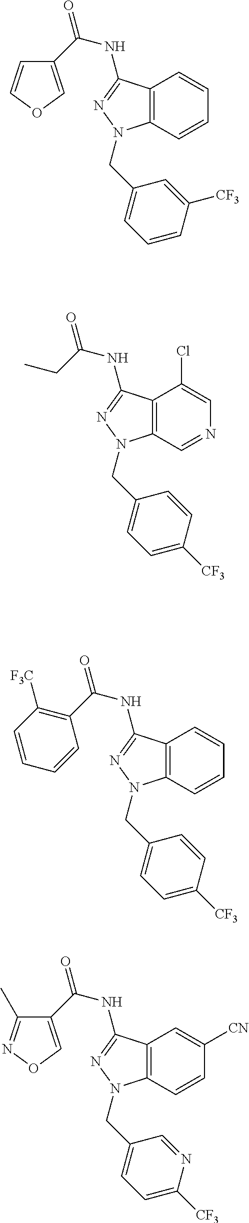 [SMILES: O=C(Nc1nn(Cc2cccc(C(F)(F)F)c2)c2ccccc12)c1ccoc1 ; CCC(=O)Nc1nn(Cc2ccc(C(F)(F)F)cc2)c2cncc(Cl)c12 ; O=C(Nc1nn(Cc2ccc(C(F)(F)F)cc2)c2ccccc12)c1ccccc1C(F)(F)F ; Cc1nocc1C(=O)Nc1nn(Cc2ccc(C(F)(F)F)nc2)c2ccc(C#N)cc12]

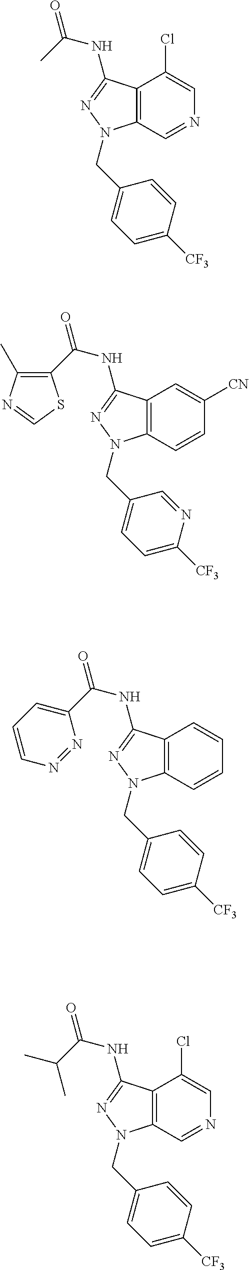 [SMILES: CC(=O)Nc1nn(Cc2ccc(C(F)(F)F)cc2)c2cncc(Cl)c12 ; Cc1ncsc1C(=O)Nc1nn(Cc2ccc(C(F)(F)F)nc2)c2ccc(C#N)cc12 ; O=C(Nc1nn(Cc2ccc(C(F)(F)F)cc2)c2ccccc12)c1cccnn1 ; CC(C)C(=O)Nc1nn(Cc2ccc(C(F)(F)F)cc2)c2cncc(Cl)c12]

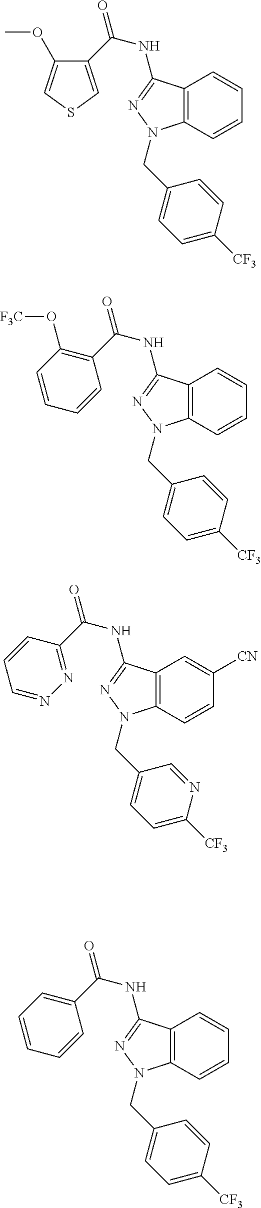 [SMILES: COc1cscc1C(=O)Nc1nn(Cc2ccc(C(F)(F)F)cc2)c2ccccc12 ; O=C(Nc1nn(Cc2ccc(C(F)(F)F)cc2)c2ccccc12)c1ccccc1OC(F)(F)F ; N#Cc1ccc2c(c1)c(NC(=O)c1cccnn1)nn2Cc1ccc(C(F)(F)F)nc1 ; O=C(Nc1nn(Cc2ccc(C(F)(F)F)cc2)c2ccccc12)c1ccccc1]

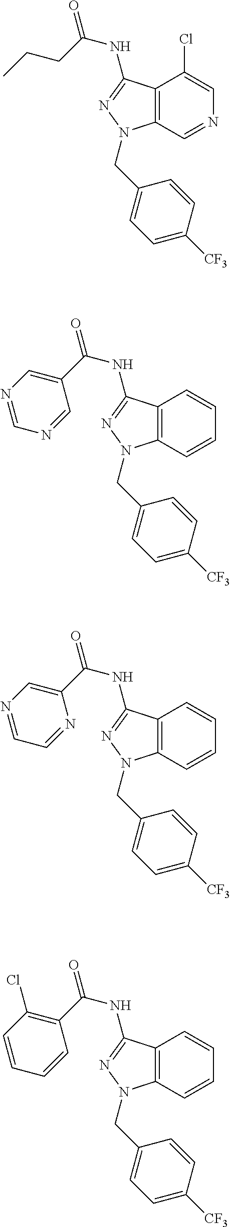 [SMILES: CCCC(=O)Nc1nn(Cc2ccc(C(F)(F)F)cc2)c2cncc(Cl)c12 ; O=C(Nc1nn(Cc2ccc(C(F)(F)F)cc2)c2ccccc12)c1cncnc1 ; O=C(Nc1nn(Cc2ccc(C(F)(F)F)cc2)c2ccccc12)c1cnccn1 ; O=C(Nc1nn(Cc2ccc(C(F)(F)F)cc2)c2ccccc12)c1ccccc1Cl]

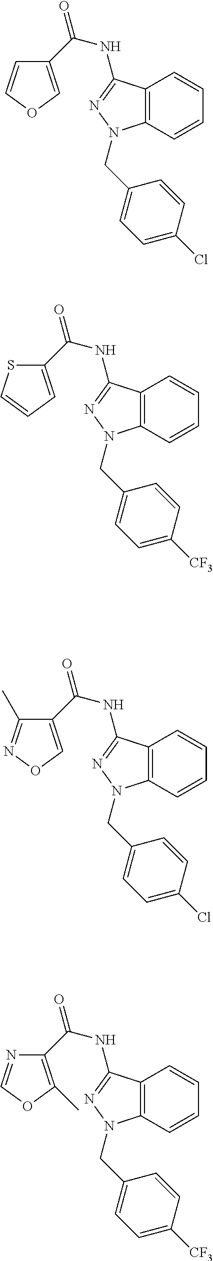 [SMILES: O=C(Nc1nn(Cc2ccc(Cl)cc2)c2ccccc12)c1ccoc1 ; O=C(Nc1nn(Cc2ccc(C(F)(F)F)cc2)c2ccccc12)c1cccs1 ; Cc1nocc1C(=O)Nc1nn(Cc2ccc(Cl)cc2)c2ccccc12 ; Cc1ocnc1C(=O)Nc1nn(Cc2ccc(C(F)(F)F)cc2)c2ccccc12]

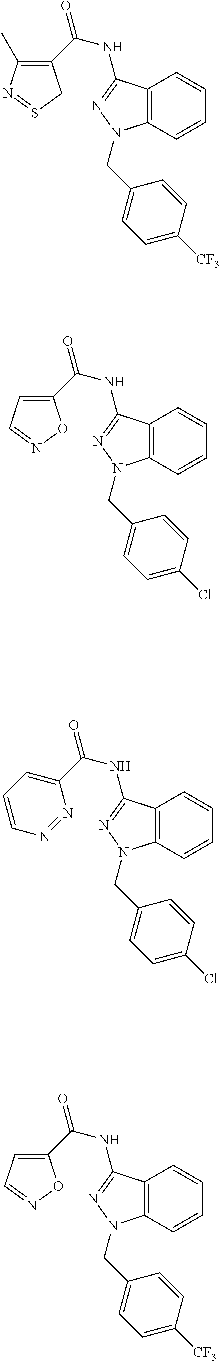

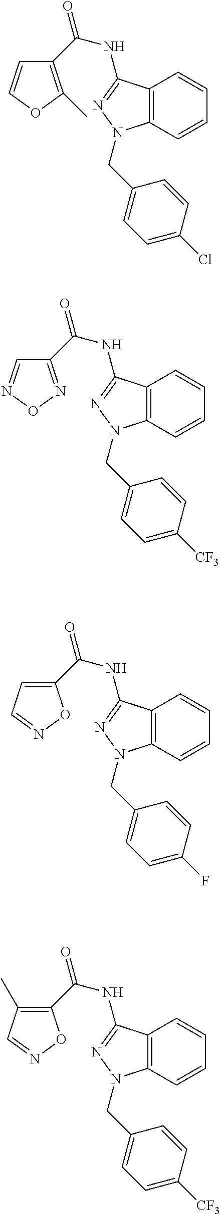 [SMILES: Cc1occc1C(=O)Nc1nn(Cc2ccc(Cl)cc2)c2ccccc12 ; O=C(Nc1nn(Cc2ccc(C(F)(F)F)cc2)c2ccccc12)c1cnon1 ; O=C(Nc1nn(Cc2ccc(F)cc2)c2ccccc12)c1ccno1 ; Cc1cnoc1C(=O)Nc1nn(Cc2ccc(C(F)(F)F)cc2)c2ccccc12]

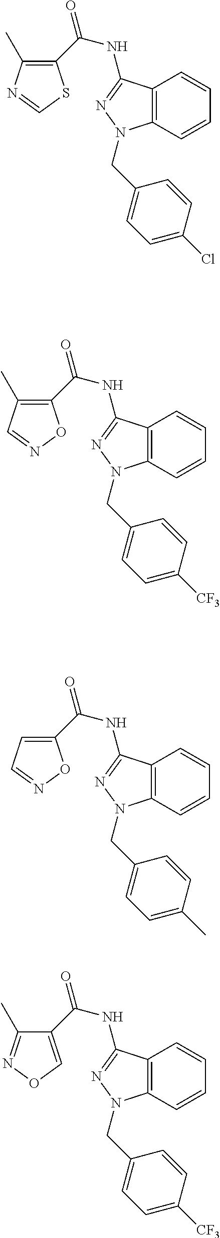 [SMILES: Cc1ncsc1C(=O)Nc1nn(Cc2ccc(Cl)cc2)c2ccccc12 ; Cc1cnoc1C(=O)Nc1nn(Cc2ccc(C(F)(F)F)cc2)c2ccccc12 ; Cc1ccc(Cn2nc(NC(=O)c3ccno3)c3ccccc32)cc1 ; Cc1nocc1C(=O)Nc1nn(Cc2ccc(C(F)(F)F)cc2)c2ccccc12]

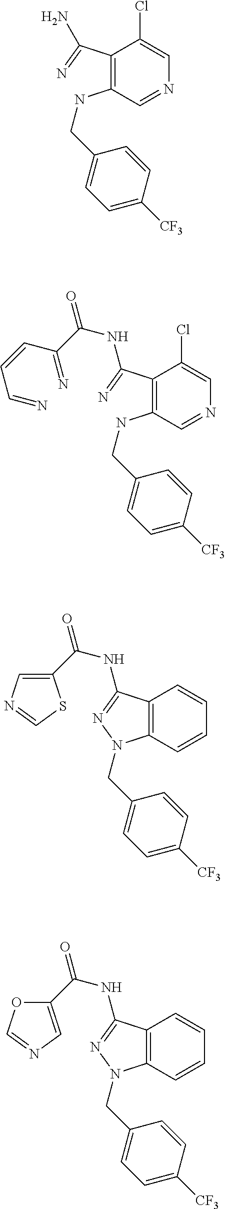 [SMILES: N=C(N)c1c(Cl)cncc1NCc1ccc(C(F)(F)F)cc1 ; N=C/C=C\C(=N)C(=O)NC(=N)c1c(Cl)cncc1NCc1ccc(C(F)(F)F)cc1 ; O=C(Nc1nn(Cc2ccc(C(F)(F)F)cc2)c2ccccc12)c1cncs1 ; O=C(Nc1nn(Cc2ccc(C(F)(F)F)cc2)c2ccccc12)c1cnco1]

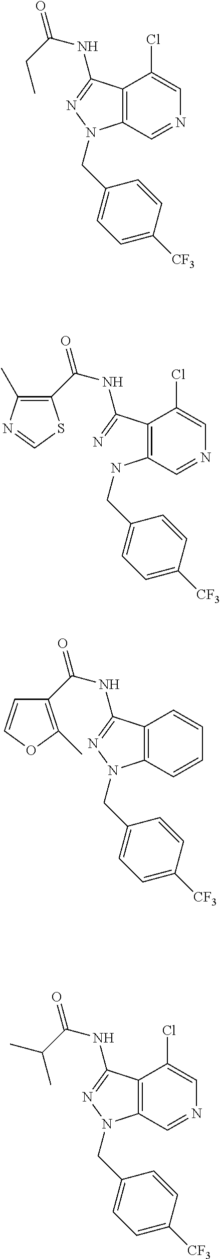 [SMILES: CCC(=O)Nc1nn(Cc2ccc(C(F)(F)F)cc2)c2cncc(Cl)c12 ; Cc1ncsc1C(=O)NC(=N)c1c(Cl)cncc1NCc1ccc(C(F)(F)F)cc1 ; Cc1occc1C(=O)Nc1nn(Cc2ccc(C(F)(F)F)cc2)c2ccccc12 ; CC(C)C(=O)Nc1nn(Cc2ccc(C(F)(F)F)cc2)c2cncc(Cl)c12]

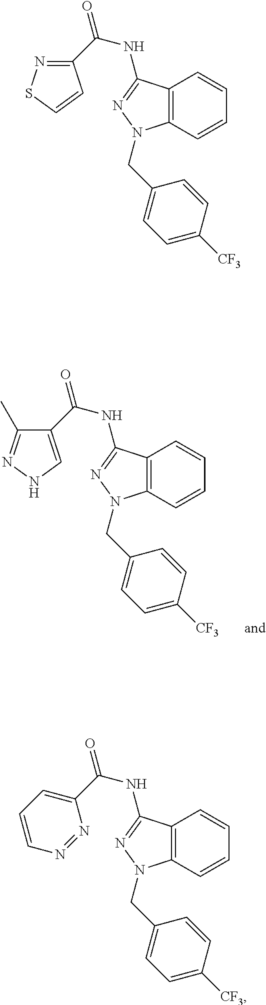 [SMILES: O=C(Nc1nn(Cc2ccc(C(F)(F)F)cc2)c2ccccc12)c1ccsn1 ; Cc1n[nH]cc1C(=O)Nc1nn(Cc2ccc(C(F)(F)F)cc2)c2ccccc12 ; O=C(Nc1nn(Cc2ccc(C(F)(F)F)cc2)c2ccccc12)c1cccnn1]

or a pharmaceutically acceptable salt thereof.

In some embodiments, the compound is selected from:

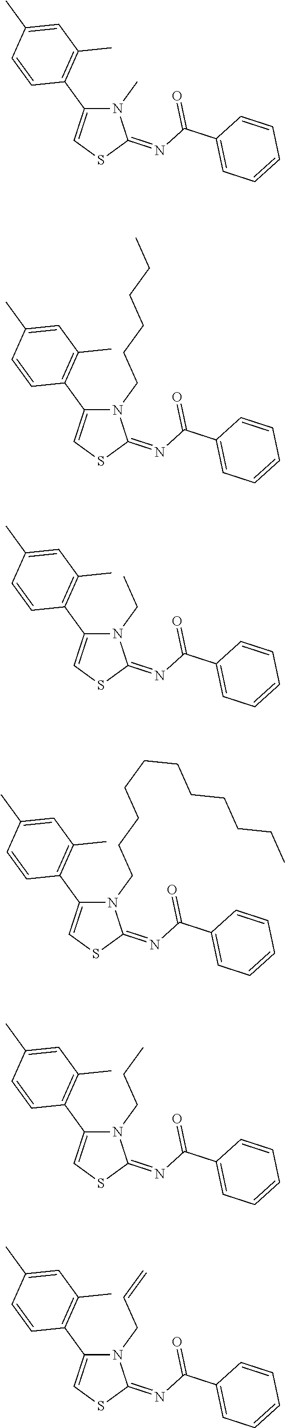

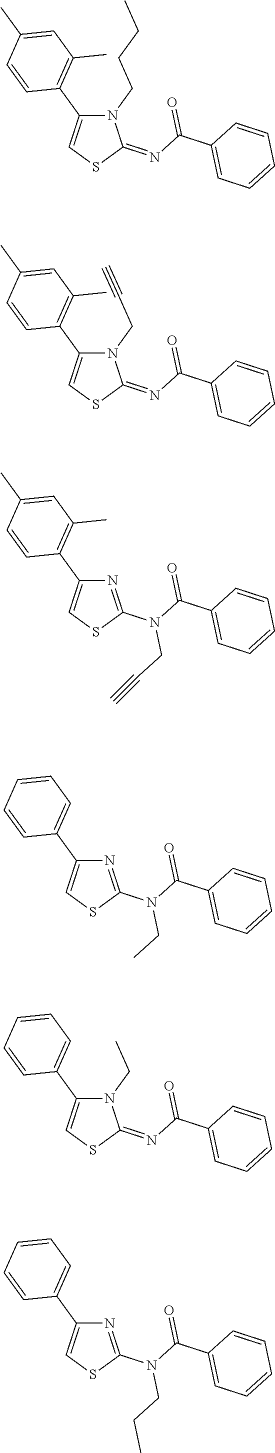 [SMILES: CCCCn1c(-c2ccc(C)cc2C)cs/c1=N/C(=O)c1ccccc1 ; C#CCn1c(-c2ccc(C)cc2C)cs/c1=N/C(=O)c1ccccc1 ; C#CCN(C(=O)c1ccccc1)c1nc(-c2ccc(C)cc2C)cs1 ; CCN(C(=O)c1ccccc1)c1nc(-c2ccccc2)cs1 ; CCn1c(-c2ccccc2)cs/c1=N/C(=O)c1ccccc1 ; CCCN(C(=O)c1ccccc1)c1nc(-c2ccccc2)cs1]

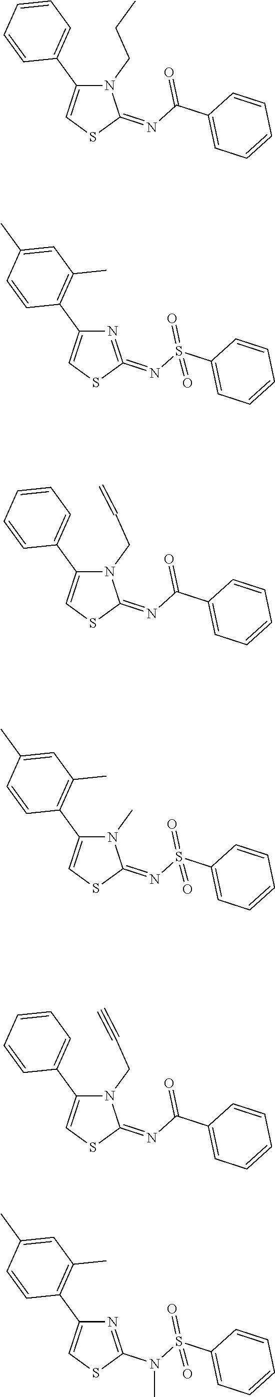

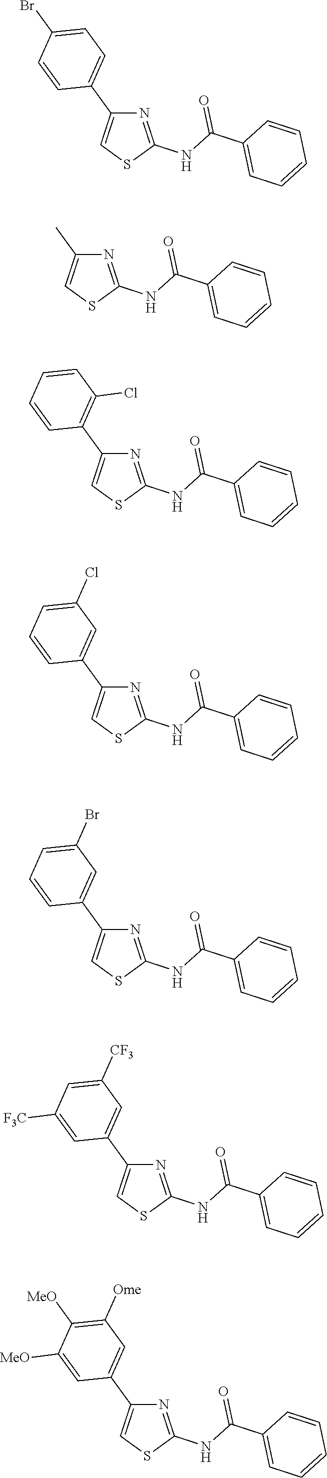

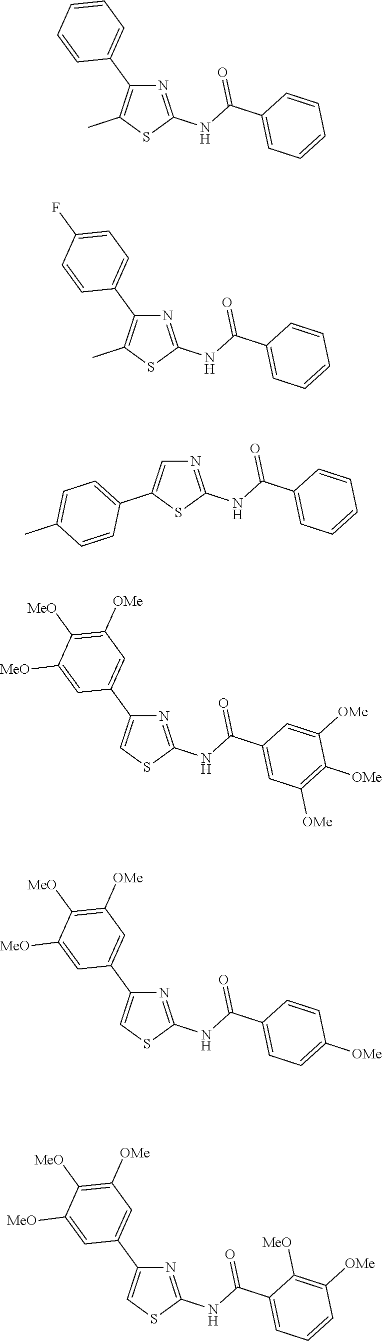 [SMILES: Cc1sc(NC(=O)c2ccccc2)nc1-c1ccccc1 ; Cc1sc(NC(=O)c2ccccc2)nc1-c1ccc(F)cc1 ; Cc1ccc(-c2cnc(NC(=O)c3ccccc3)s2)cc1 ; COc1cc(C(=O)Nc2nc(-c3cc(OC)c(OC)c(OC)c3)cs2)cc(OC)c1OC ; COc1ccc(C(=O)Nc2nc(-c3cc(OC)c(OC)c(OC)c3)cs2)cc1 ; COc1cc(-c2csc(NC(=O)c3cccc(OC)c3OC)n2)cc(OC)c1OC]

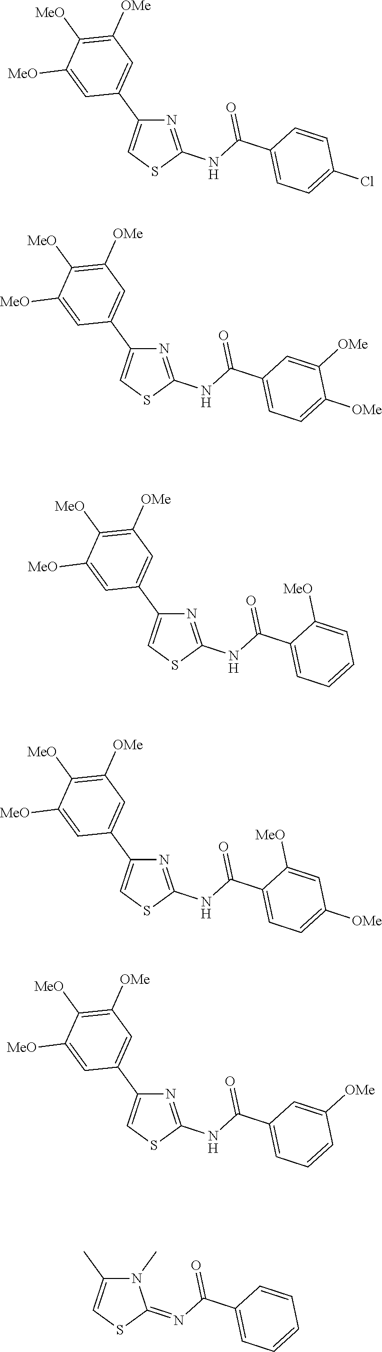 [SMILES: COc1cc(-c2csc(NC(=O)c3ccc(Cl)cc3)n2)cc(OC)c1OC ; COc1ccc(C(=O)Nc2nc(-c3cc(OC)c(OC)c(OC)c3)cs2)cc1OC ; COc1ccccc1C(=O)Nc1nc(-c2cc(OC)c(OC)c(OC)c2)cs1 ; COc1ccc(C(=O)Nc2nc(-c3cc(OC)c(OC)c(OC)c3)cs2)c(OC)c1 ; COc1cccc(C(=O)Nc2nc(-c3cc(OC)c(OC)c(OC)c3)cs2)c1 ; Cc1cs/c(=N/C(=O)c2ccccc2)n1C]

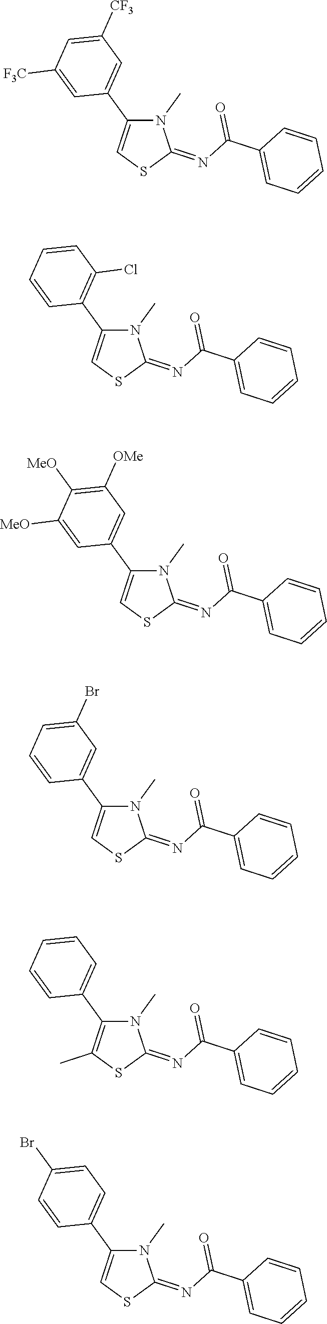 [SMILES: Cn1c(-c2cc(C(F)(F)F)cc(C(F)(F)F)c2)cs/c1=N/C(=O)c1ccccc1 ; Cn1c(-c2ccccc2Cl)cs/c1=N/C(=O)c1ccccc1 ; COc1cc(-c2cs/c(=N/C(=O)c3ccccc3)n2C)cc(OC)c1OC ; Cn1c(-c2cccc(Br)c2)cs/c1=N/C(=O)c1ccccc1 ; Cc1s/c(=N/C(=O)c2ccccc2)n(C)c1-c1ccccc1 ; Cn1c(-c2ccc(Br)cc2)cs/c1=N/C(=O)c1ccccc1]

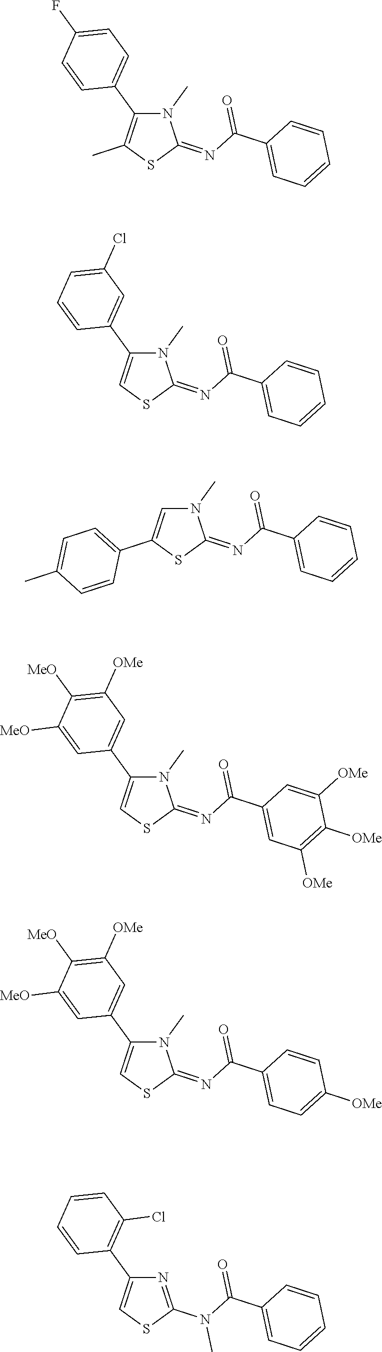 [SMILES: Cc1s/c(=N/C(=O)c2ccccc2)n(C)c1-c1ccc(F)cc1 ; Cn1c(-c2cccc(Cl)c2)cs/c1=N/C(=O)c1ccccc1 ; Cc1ccc(-c2cn(C)/c(=N\C(=O)c3ccccc3)s2)cc1 ; COc1cc(C(=O)/N=c2/scc(-c3cc(OC)c(OC)c(OC)c3)n2C)cc(OC)c1OC ; COc1ccc(C(=O)/N=c2/scc(-c3cc(OC)c(OC)c(OC)c3)n2C)cc1 ; CN(C(=O)c1ccccc1)c1nc(-c2ccccc2Cl)cs1]

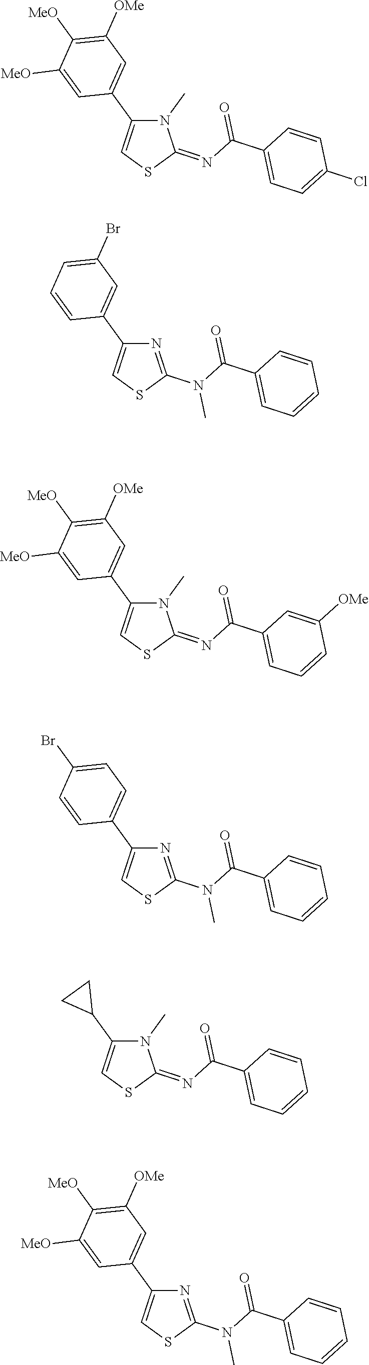 [SMILES: COc1cc(-c2cs/c(=N/C(=O)c3ccc(Cl)cc3)n2C)cc(OC)c1OC ; CN(C(=O)c1ccccc1)c1nc(-c2cccc(Br)c2)cs1 ; COc1cccc(C(=O)/N=c2/scc(-c3cc(OC)c(OC)c(OC)c3)n2C)c1 ; CN(C(=O)c1ccccc1)c1nc(-c2ccc(Br)cc2)cs1 ; Cn1c(C2CC2)cs/c1=N/C(=O)c1ccccc1 ; COc1cc(-c2csc(N(C)C(=O)c3ccccc3)n2)cc(OC)c1OC]

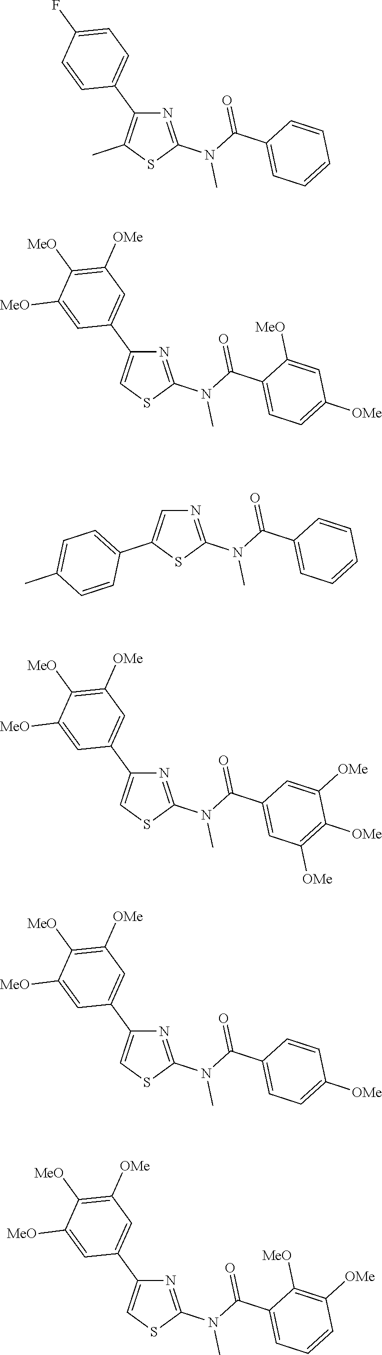 [SMILES: Cc1sc(N(C)C(=O)c2ccccc2)nc1-c1ccc(F)cc1 ; COc1ccc(C(=O)N(C)c2nc(-c3cc(OC)c(OC)c(OC)c3)cs2)c(OC)c1 ; Cc1ccc(-c2cnc(N(C)C(=O)c3ccccc3)s2)cc1 ; COc1cc(C(=O)N(C)c2nc(-c3cc(OC)c(OC)c(OC)c3)cs2)cc(OC)c1OC ; COc1ccc(C(=O)N(C)c2nc(-c3cc(OC)c(OC)c(OC)c3)cs2)cc1 ; COc1cc(-c2csc(N(C)C(=O)c3cccc(OC)c3OC)n2)cc(OC)c1OC]

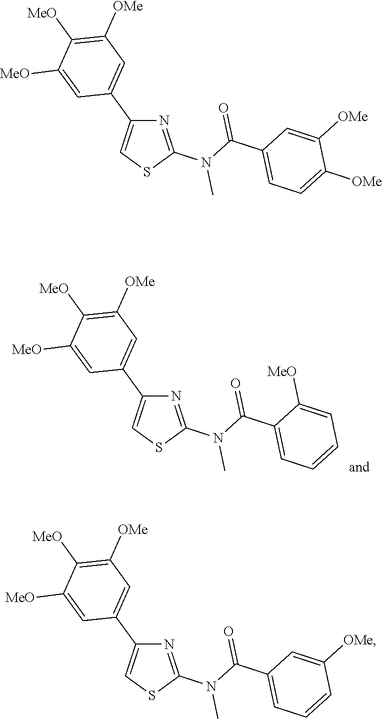 [SMILES: COc1ccc(C(=O)N(C)c2nc(-c3cc(OC)c(OC)c(OC)c3)cs2)cc1OC ; COc1ccccc1C(=O)N(C)c1nc(-c2cc(OC)c(OC)c(OC)c2)cs1 ; COc1cccc(C(=O)N(C)c2nc(-c3cc(OC)c(OC)c(OC)c3)cs2)c1]

or a pharmaceutically acceptable salt thereof.

In some embodiments, the compound is selected from:

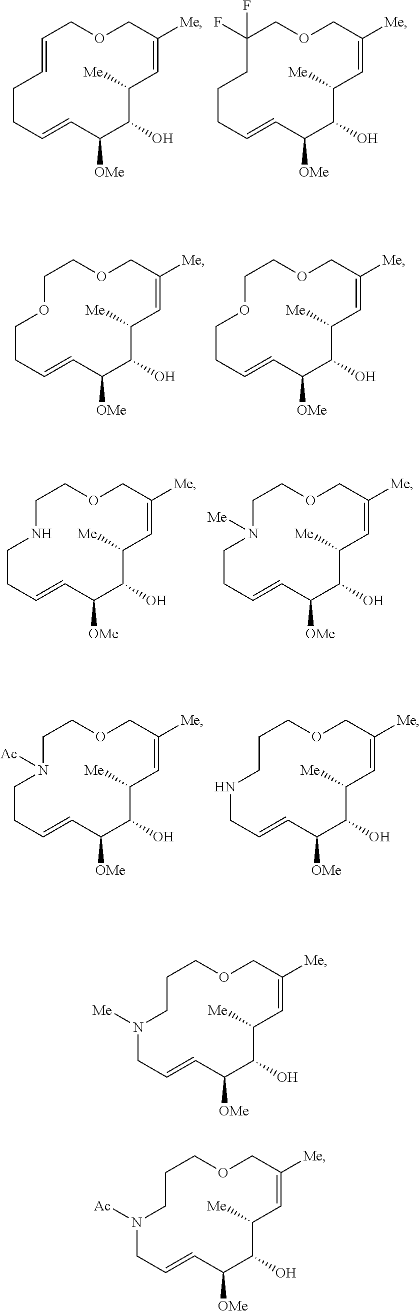

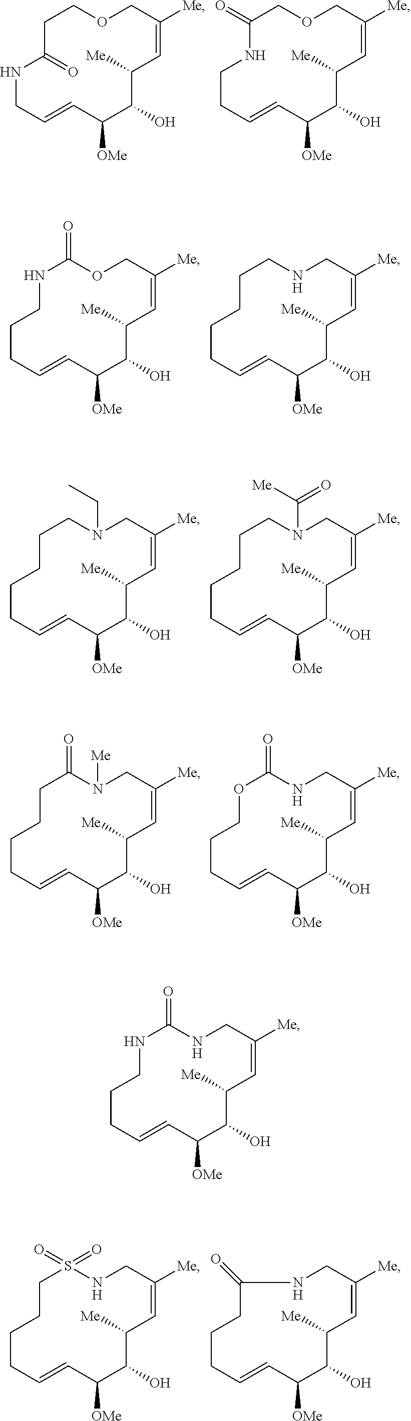

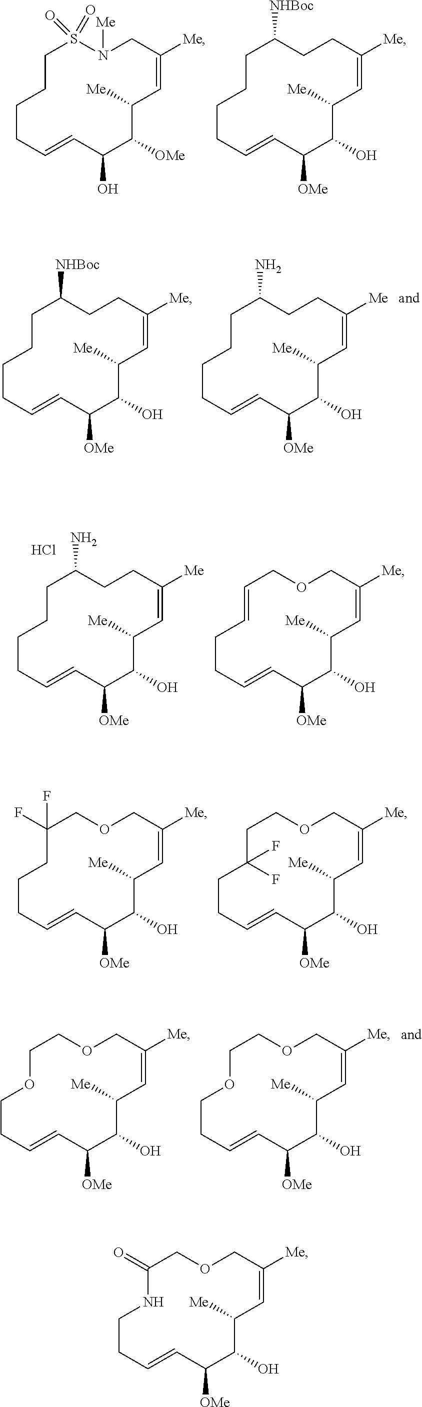

or a pharmaceutically acceptable salt thereof.

In some embodiments, the compound is selected from

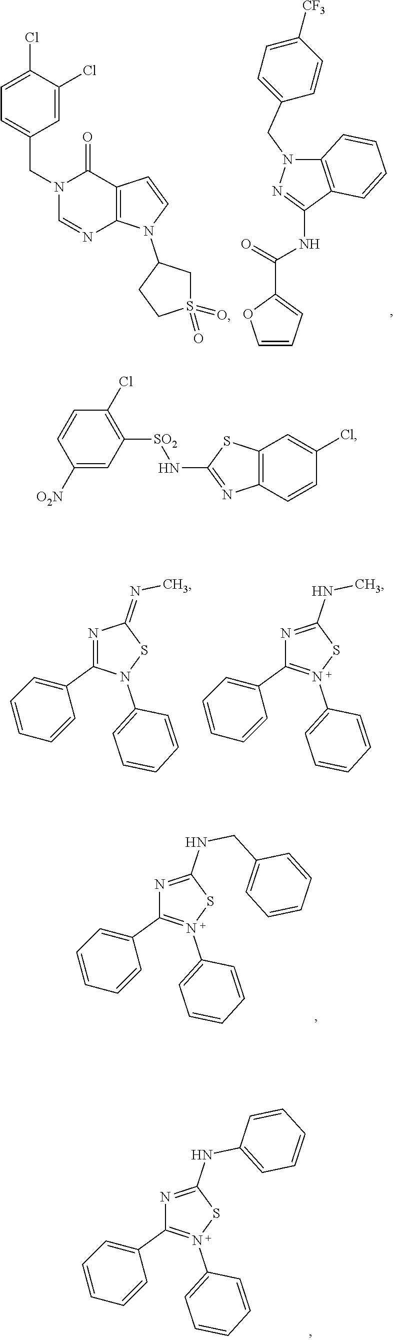

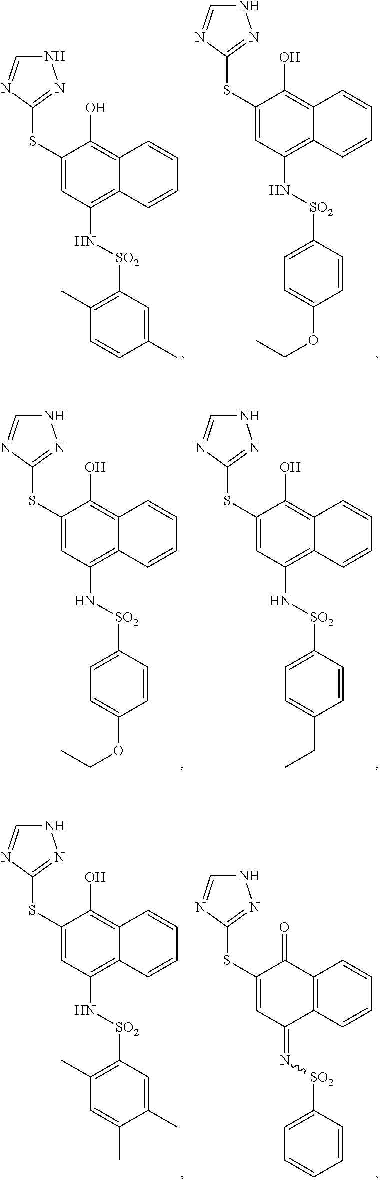

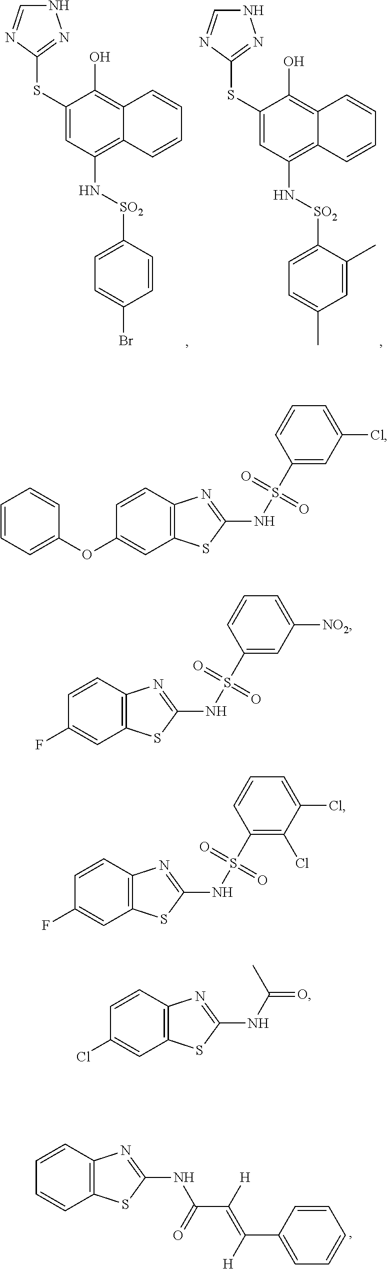

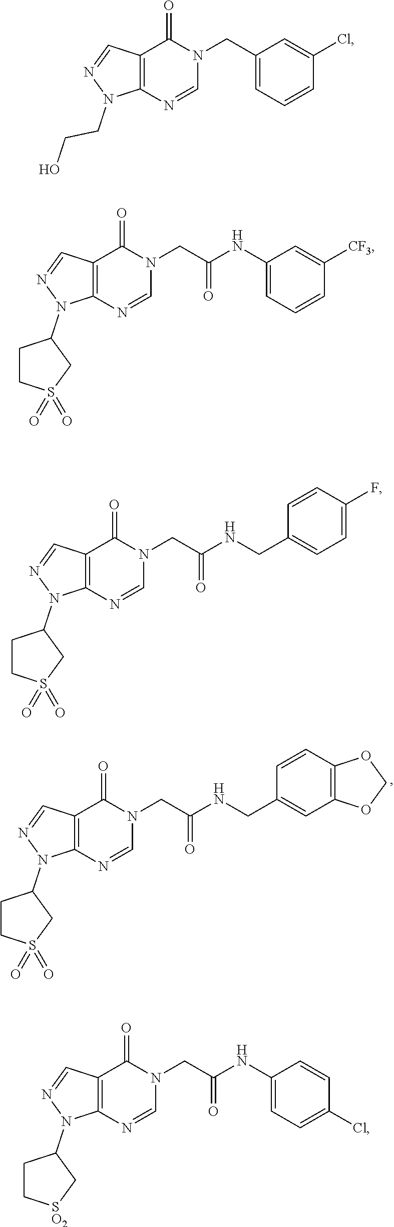 [SMILES: O=c1c2cnn(CCO)c2ncn1Cc1cccc(Cl)c1 ; O=C(Cn1cnc2c(cnn2C2CCS(=O)(=O)C2)c1=O)Nc1cccc(C(F)(F)F)c1 ; O=C(Cn1cnc2c(cnn2C2CCS(=O)(=O)C2)c1=O)NCc1ccc(F)cc1 ; O=C(Cn1cnc2c(cnn2C2CCS(=O)(=O)C2)c1=O)NCc1ccc2c(c1)OCO2 ; O=C(Cn1cnc2c(cnn2C2CCS(=O)(=O)C2)c1=O)Nc1ccc(Cl)cc1]

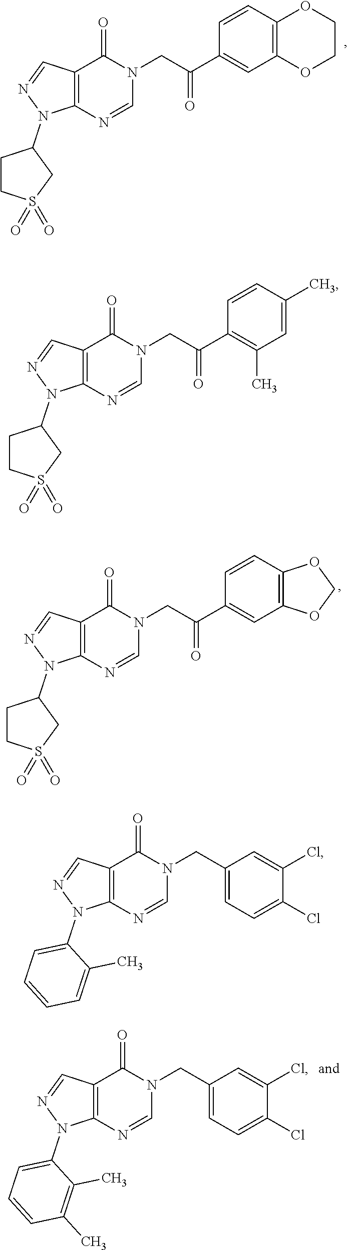 [SMILES: O=C(Cn1cnc2c(cnn2C2CCS(=O)(=O)C2)c1=O)c1ccc2c(c1)OCCO2 ; Cc1ccc(C(=O)Cn2cnc3c(cnn3C3CCS(=O)(=O)C3)c2=O)c(C)c1 ; O=C(Cn1cnc2c(cnn2C2CCS(=O)(=O)C2)c1=O)c1ccc2c(c1)OCO2 ; Cc1ccccc1-n1ncc2c(=O)n(Cc3ccc(Cl)c(Cl)c3)cnc21 ; Cc1cccc(-n2ncc3c(=O)n(Cc4ccc(Cl)c(Cl)c4)cnc32)c1C ; "and"]

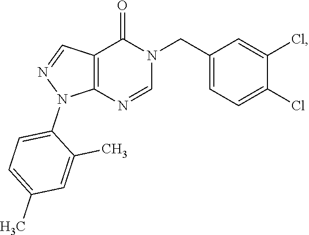 [SMILES: Cc1ccc(-n2ncc3c(=O)n(Cc4ccc(Cl)c(Cl)c4)cnc32)c(C)c1]

or a pharmaceutically acceptable salt thereof.

In some embodiments, the compound is

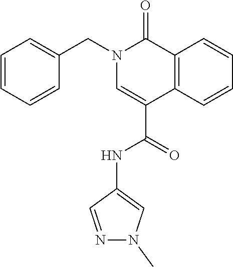 [SMILES: Cn1cc(NC(=O)c2cn(Cc3ccccc3)c(=O)c3ccccc23)cn1]

or a pharmaceutically acceptable salt thereof.

In some embodiments, the compound is N-(1-(4-(trifluoromethyl)benzyl)-1H-indazol-3-yl)furan-2-carboxamide, having the structure:

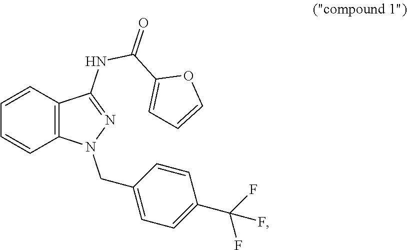 [SMILES: O=C(Nc1nn(Cc2ccc(C(F)(F)F)cc2)c2ccccc12)c1ccco1]

or a pharmaceutically acceptable salt thereof.

In some embodiments, the compound is not comparative compound 2, comparative compound 3, or comparative compound 4:

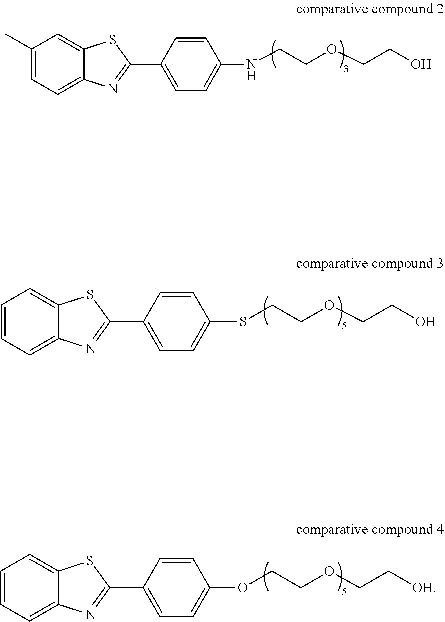

In some embodiments, comparative compound 2, comparative compound 3, or comparative compound 4 is present as a pharmaceutically acceptable salt thereof.

In some embodiments, the compound is compound 5 or compound 6:

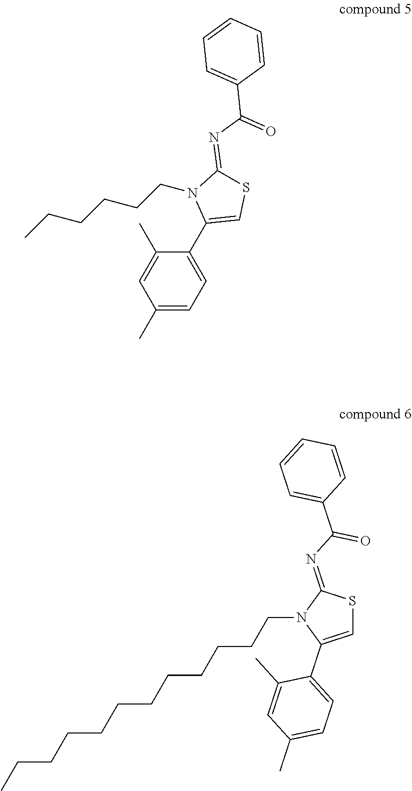 [SMILES: CCCCCCn1c(-c2ccc(C)cc2C)cs/c1=N\C(=O)c1ccccc1 ; CCCCCCCCCCCCn1c(-c2ccc(C)cc2C)cs/c1=N\C(=O)c1ccccc1]

or a pharmaceutically acceptable salt thereof.

In some embodiments, the compound is selected from:

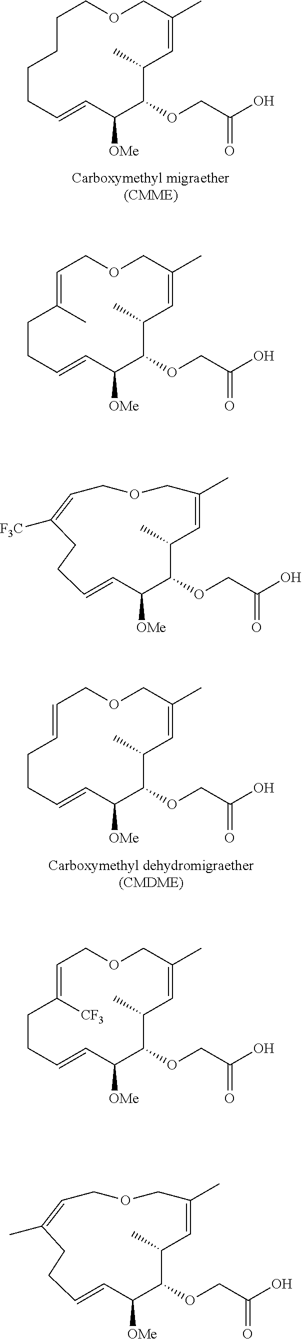 [SMILES: CO[C@H]1/C=C/CCCCCOC/C(C)=C\[C@@H](C)[C@@H]1OCC(=O)O ; CO[C@H]1/C=C/CC/C(C)=C/COC/C(C)=C\[C@@H](C)[C@@H]1OCC(=O)O ; CO[C@H]1/C=C/CC/C(C(F)(F)F)=C\COC/C(C)=C\[C@@H](C)[C@@H]1OCC(=O)O ; CO[C@H]1/C=C/CC/C=C/COC/C(C)=C\[C@@H](C)[C@@H]1OCC(=O)O ; CO[C@H]1/C=C/CC/C(C(F)(F)F)=C/COC/C(C)=C\[C@@H](C)[C@@H]1OCC(=O)O ; CO[C@H]1/C=C/CC/C(C)=C\COC/C(C)=C\[C@@H](C)[C@@H]1OCC(=O)O]

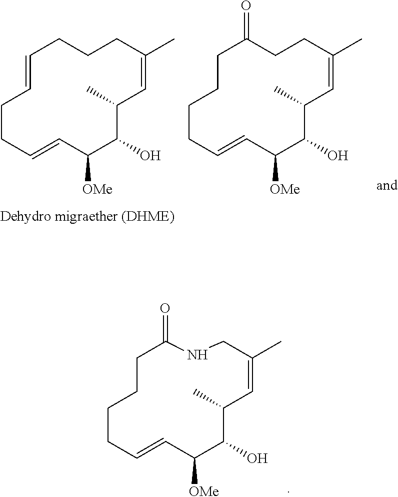

In some embodiments, the compound is migrastatin (compound 8) or isomigrastatin (compound 9):

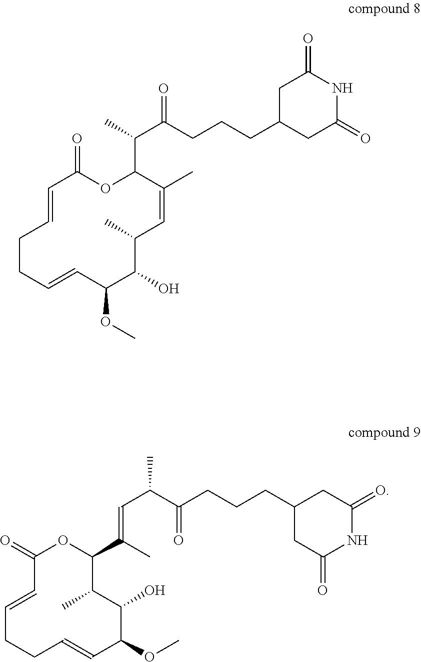 [SMILES: CO[C@H]1/C=C/CC/C=C/C(=O)OC([C@H](C)C(=O)CCCC2CC(=O)NC(=O)C2)/C(C)=C\[C@@H](C)[C@@H]1O ; CO[C@H]1/C=C/CC/C=C/C(=O)O[C@@H](/C(C)=C/[C@H](C)C(=O)CCCC2CC(=O)NC(=O)C2)[C@@H](C)[C@@H]1O]

In some embodiments, compound 8 or compound 9 is present as a pharmaceutically acceptable salt thereof.

In some embodiments, the compound is compound 10:

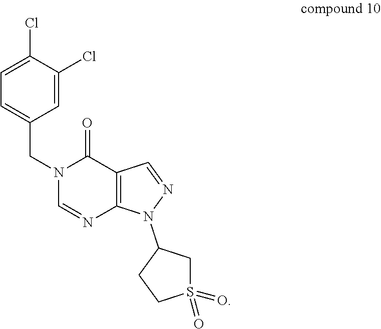 [SMILES: O=c1c2cnn(C3CCS(=O)(=O)C3)c2ncn1Cc1ccc(Cl)c(Cl)c1]

In some embodiments, compound 10 is present as a pharmaceutically acceptable salt thereof.

In some embodiments, the compound is compound 11:

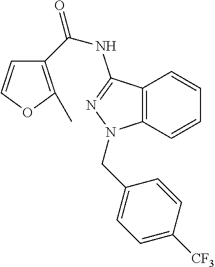 [SMILES: Cc1occc1C(=O)Nc1nn(Cc2ccc(C(F)(F)F)cc2)c2ccccc12]

or a pharmaceutically acceptable salt thereof.

In some embodiments, the compound is BDP-00010834 or BDP-00013544 as disclosed in Abstract LB-228: Identifying small molecule inhibitors of Fascin 1 using fragment-based drug discovery, Cancer Research 77(13 Supplement):LB-228-LB-228, July 2017; DOI: 10.1158/1538-7445.AM2017-LB-228.

In some embodiments, the agent is a fascin antibody that binds to fascin at least at binding site 2 or binding site 3.

In some embodiments, the compound is not a compound of formula III:

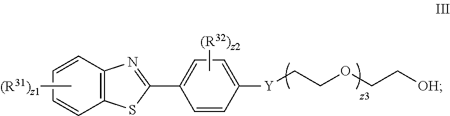

wherein Y is —NR³³— or —S—; R³¹ is independently halogen, —CX³¹, —CHX³¹, —CH₂X³¹, —OCX³¹₃, —OCHX³¹₂, —OCH₂X³¹, —CN, —OH, —NH₂, —COOH, —CONH₂, —NO₂, —SH, —SO₃H, —SO₄H, —SO₂NH₂, —NHNH₂, —ONH₂, —HNC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocyclyl, substituted or unsubstituted aryl, or substituted or unsubstituted heteroaryl; R³² is independently halogen, —CX³²₃, —CHX³²₂, —CH₂X³², —OCX³²₃, —OCHX³²₂, —OCH₂X³², —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocyclyl, substituted or unsubstituted aryl, or substituted or unsubstituted heteroaryl; each of X³¹ and X³² are independently halogen; each of z1 and z2 is independently an integer from 0 to 4; z3 is an integer from 1 to 12; and R³³ is hydrogen or substituted or unsubstituted C₁₋₆ alkyl.

Compounds described herein can be prepared according to methods known to those of skill in the art. If available, compounds may be purchased commercially, e.g., from Sigma Aldrich or other chemical suppliers.

Synthesis can be conducted by known procedures or modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures or modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplemental (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### Treatment Methods and Uses

### Fascin

Described herein are methods for the regeneration of spine synapses lost to neurodegenerative conditions by targeting a cytoskeletal protein with an agent such as a compound described herein. Unexpectedly, it was observed that inhibition of the cytoskeletal protein fascin 1 (FSCN1) resulted in a rapid upregulation of dendritic spines in vivo and in vitro. Dendritic spines contain filamentous actin (F-actin), a cytoskeletal polymer that gives structure to cells and their subcellular specializations. Extension of F-actin filaments and changes in their organization are believed to be important for the formation, maturation, and plasticity of dendritic spines. Prior to the present disclosure, it was believed that the ability of fascin 1 to bundle F-actin filaments into parallel arrays would be essential for the formation of a wide range of cellular protrusions, such as invadopodia, filapodia and possibly dendritic spines. Fascin 1 is believed to promote cellular migration and the related process of cancer metastasis by such processes. Small molecule inhibitors of fascin 1 that block its ability to bundle F-actin have been observed to reduce F-actin-rich cellular protrusions. Thus, prior work suggested that fascin inhibitors would also block the formation of dendritic spines, which are cellular protrusions rich in F-actin. However, contrary to expectation, the present disclosure demonstrates that the opposite is true: that structurally distinct inhibitors of fascin 1, as well as genetic knockdown of fascin 1, may result in a rapid increase in dendritic spine density. Without wishing to be limited by theory, it is believed that dendritic spines require the formation of highly branched assemblies of F-actin, and formation of such assemblies may be precluded, or significantly reduced, by bundling into parallel arrays by fascin 1.

In some embodiments, a method of binding a fascin protein at site 2 or site 3 is provided, the method comprising contacting the fascin protein with an effective amount of a compound described herein, for example, a compound of formula I as provided herein, a pharmaceutically acceptable salt thereof, or a compound of formula II as provided herein, or a pharmaceutically acceptable salt thereof, a compound selected from a compound of formula IV as provided herein, or a pharmaceutically acceptable salt thereof, a compound selected from a compound of formula V as provided herein, or a pharmaceutically acceptable salt thereof, a compound selected from a compound of formula VII as provided herein, or a pharmaceutically acceptable salt thereof, or compound 1, compound 8, compound 9, compound 10, or compound 11 or a pharmaceutically acceptable salt thereof. In some embodiments, the method inhibits fascin. It is believed that a compound of formula I, formula II, formula IV, formula V, formula VII, formula VIII, formula IX, formula X, or formula XI, as provided herein, or a pharmaceutically acceptable salt thereof, or compound 1, compound 8, compound 9, compound 10, or compound 11 or a pharmaceutically acceptable salt thereof, promote dendritic spine formation by inhibiting fascin.

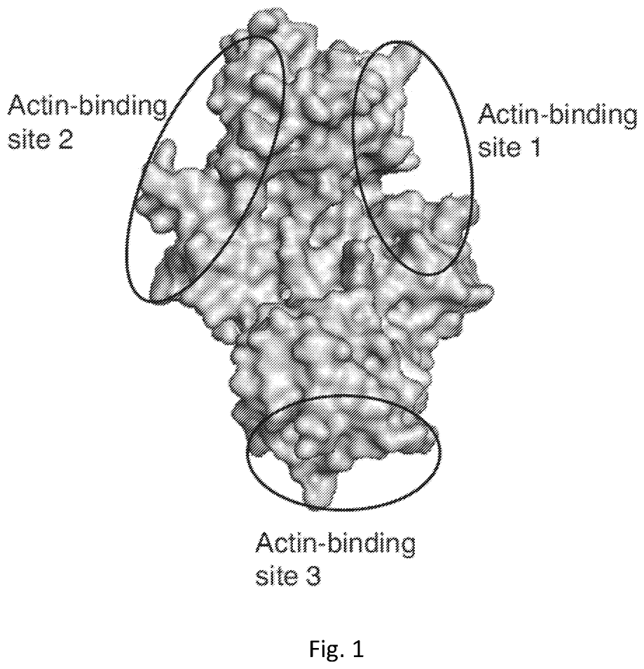

Fascin is an important actin cross-linker that has no amino-acid sequence homology with other actin-binding proteins. Three forms of fascin are found in vertebrates: fascin 1, widely found in the nervous system and elsewhere; fascin 2 found in the retinal photoreceptor cells; and fascin 3, which is only found in the testes. In some embodiments, a fascin is human fascin 1. Fascin has a molecular mass of 55 kDa, functions as a monomeric entity, and cross-links actin filaments into straight, compact and rigid bundles, to impart mechanical stiffness to actin bundles. It is believed that fascin holds parallel actin filaments together to form filopodia on the order of 60-200 nm in diameter. A structure of fascin, along with actin binding sites, is illustrated in FIG. 1.

During neuron development, it is believed that long bundles of f-actin push out the membrane of the neuron to form structures such as axons, dendrites, filopodia and lamellipodia. Fascin is thought to be involved in cytoskeletal reorganization of nascent dendritic protrusions. Thus, actin bundling by fascin is generally believed to be required for the formation and extension of axons and dendrites. Surprisingly, the present results indicate that inhibiting the activity of fascin in formation of actin bundles promotes the formation of dendritic spines, protrusions of the cytoplasmic membrane of dendrites.

Fascin is believed to have at least three binding sites, binding site 1, binding site 2 and binding site 3. Thus, with reference to FIG. 1, fascin appears to have three sites at which actin may be bound. Binding site 2 was not seen in early apo (ligand-free) crystal structures of fascin, perhaps due to movement of the protein structure upon ligand binding. Compounds disclosed in International Patent Publication No. WO 2017/120198 are believed to bind fascin at binding site 1.

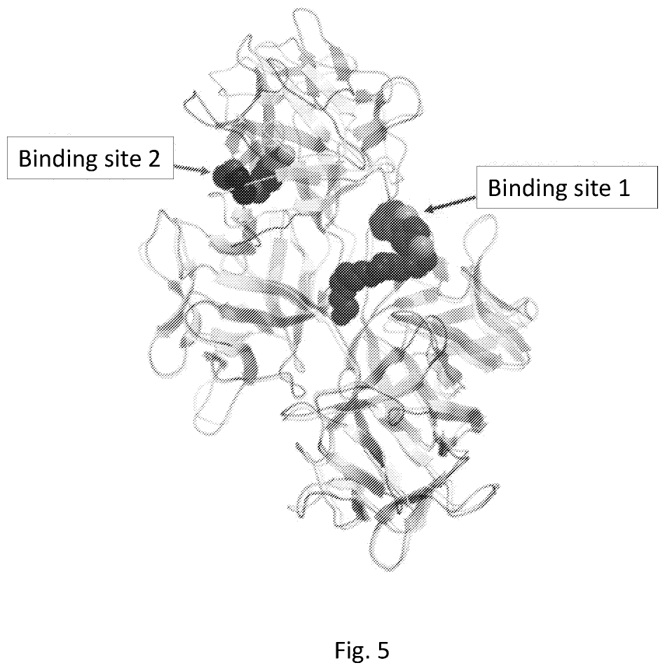

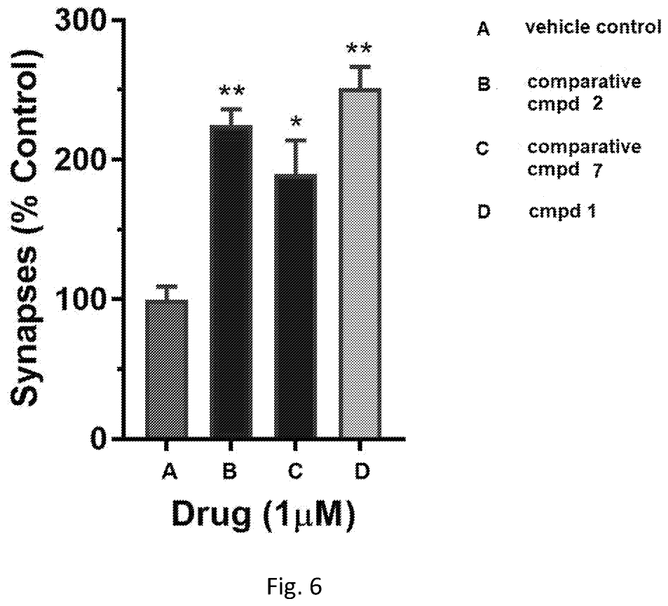

It has been observed that when compound 1 binds it opens actin-binding site 2 and closes actin-binding site 1, preventing, at least in part, the bundling of actin filaments. Compound 1 has been found to bind to fascin at binding site 2, as illustrated in FIG. 5. Further, it has been discovered that compound 1 may increase spine density compared to control. FIG. 6. In particular, compound 1 was found to increase spine density more than comparative compounds 1 and 7. FIG. 6. Thus, a new molecular pathway for increasing dendritic spine density by inhibiting fascin by binding at least at binding site 2 is contemplated.

In some embodiments, fascin binding site 1 is defined, at least in part, by V10, Q11, L40, K41, A137, H139, Q141, Q258, S259, R383, R389, E391, G393, F394, S409, Y458, K460, E492, and/or Y493. In some embodiments, an agent described herein, such as a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, does not bind to any of fascin residue selected from V10, Q11, L40, K41, A137, H139, Q141, Q258, S259, R383, R389, E391, G393, F394, S409, Y458, K460, E492, and/or Y493. In some embodiments, an agent described herein, such as a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, does not bind to fascin binding site 1 and binding site 2. In some embodiments, an agent described herein, such as a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, does not bind to fascin binding site 1 and binding site 3. In some embodiments, an agent described herein, such as a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, does not bind to fascin binding site 1, binding site 2, and binding site 3.

Fascin binding site 2 is believed to be defined, at least in part, by F14, L16, L48, Q50, L62, W101, L103, E215, and/or S218. In some embodiments, an agent described herein, such as a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, binds to at least one fascin residue selected from F14, L16, L48, Q50, L62, W101, L103, E215, and S218. In some embodiments, an agent described herein, such as a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, binds to two, three, four, five, six, seven, or eight fascin residues selected from F14, L16, L48, Q50, L62, W101, L103, E215, and S218. In some embodiments, an agent described herein, such as a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, binds to at least one group I fascin residue selected from F14 and L16. In some embodiments, an agent described herein, such as a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, binds to at least one group n fascin residue selected from L48, Q50, and L62. In some embodiments, an agent described herein, such as a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, binds to at least one group TTT fascin residue selected from W101 and L103. In some embodiments, an agent described herein, such as a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, binds to at least one group IV fascin residue selected from E215 and S218. In some embodiments, fascin binding site 2 is defined, at least in part, by F14, L16, L48, A58, V60, L62, I93, A95, W101, L103, V134, T213, L214, E215, F216, and/or R217.

In some embodiments, fascin binding site 3 is defined, at least in part, by Q291, R308, H310, T311, G312, K313, Y314, L317, T318, T320, T326, S328, K329, N330, N331, S333, E339, R341, R344, R348, K353, S350, N351, F354, T356, S357, K358, K359, N360, Q362, L363, S366, V367, E368, T369, D372, S373, L375, L377, 1381, and/or K379. In some embodiments, an agent described herein, such as a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, binds to at least one fascin residue selected from Q291, R308, H310, T311, G312, K313, Y314, L317, T318, T320, T326, S328, K329, N330, N331, S333, E339, R341, R344, R348, K353, S350, N351, F354, T356, S357, K358, K359, N360, Q362, L363, S366, V367, E368, T369, D372, S373, L375, L377, 1381, and K379. In some embodiments, an agent described herein, such as a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, binds to two, three, four, five, six, seven, or eight fascin residues selected from Q291, R308, H310, T311, G312, K313, Y314, L317, T318, T320, T326, S328, K329, N330, N331, S333, E339, R341, R344, R348, K353, S350, N351, F354, T356, S357, K358, K359, N360, Q362, L363, S366, V367, E368, T369, D372, S373, L375, L377, 1381, and K379.

In some embodiments a binding site of an agent such as a compound described herein can be determined by site-directed mutagenesis. For example, an amino acid residue in a mutant fascin may be changed relative to a wild-type fascin. For example, if a reduction in binding affinity of an agent is determined between a wild type fascin and a mutated fascin having replacement of an amino acid residue at binding site 1, binding site 2, or binding site 3 with a non-native residue, e.g., an alanine residue, then the reduction can be attributed to a loss of affinity for binding at the site of replacement. Site-directed mutagenesis may be carried out according a known method, for example, Kunkel's method, Cassette mutagenesis, PCR site-directed mutagenesis, or CRISPR.

In some embodiments, an agent described herein binds to fascin with a K_{d} of at least about 1 μM, at least about 5 μM, at least about 10 μM, at least about 20 μM, at least about 50 μM, at least about 100 μM, or at least about 500 μM as determined by isothermal titration calorimetry.

Various small molecule compounds as disclosed herein can bind to the fascin binding site 2 or fascin binding site 3. Their variants and additional compounds can also be identified with methods known in the art. For instance, antibodies can be identified that bind to amino acid resides within the fascin binding site 2 or 3, which serve as epitopes for the antibodies.

Methods of making antibodies are well known in the art and described herein. For example, antibodies against a specific epitope on a protein can be prepared by administering the protein or the epitope fragment to an animal. Antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans.

The binding specificity of antigen-binding polypeptides of the present disclosure can be determined by in vitro assays such as immunoprecipitation, radioimmunoassay (RIA) or enzyme-linked immunoabsorbent assay (ELISA).

Humanized antibodies are antibody molecules derived from a non-human species antibody that bind the desired antigen having one or more complementarity determining regions (CDRs) from the non-human species and framework regions from a human immunoglobulin molecule. Completely human antibodies are particularly desirable for therapeutic treatment of human patients. Human antibodies can be made by a variety of methods known in the art including phage display methods using antibody libraries derived from human immunoglobulin sequences. Human antibodies can also be produced using transgenic mice which are incapable of expressing functional endogenous immunoglobulins, but which can express human immunoglobulin genes.

### Neuronal Diseases and Conditions and Treatment Thereof

A unifying feature of neurodegenerative conditions with a cognitive component is the loss of synapses that utilize the amino acid glutamate as a neurotransmitter (“glutamatergic” synapses), which in humans and other mammals are believed to be the most numerous type of synapse. Importantly, about 90% of glutamatergic synapses involve a postsynaptic dendritic spine. The majority of synapses lost in neurodegenerative conditions are those in which the axon makes contact with a dendritic spine, so-called “axospinous synapses.” Under normal conditions, changes in the density, shape, and protein composition of dendritic spines impact the strength of synaptic communication, and are the basis of several forms of synaptic change (i.e. “plasticity”) involved in learning and memory, cognitive flexibility, adaptation to injury and disease, and other processes. These changes in axospinous synapses are believed to be important for the memory encoding functions of structures such as the hippocampus. Accordingly, an early and progressive loss of dendritic spines in hippocampus and other regions is believed to be a driver of memory loss and cognitive decline in Alzheimer's disease and other dementias. The development of novel methods to regenerate spine density could have important implications for treatment of a host of neurodegenerative and developmental cognitive disorders.

Dendritic spines are specialized protrusions responsible for receiving synaptic inputs, providing an important function in communication between neurons. The morphology of dendritic spines and their overall density correlates with synaptic function and are strongly implicated in memory and learning. Cellular changes in brain cells may contribute to pathogenesis of a neuronal disease. For example, an aberrant level (e.g., reduction) in dendritic spine density in the brain may contribute to the pathogenesis of the neuronal disease. Consequently, alteration or misregulation of dendritic spines is believed to influence synaptic function and play a major role in various neurological and psychiatric disorders such as autism, fragile X syndrome, Parkinson's Disease (PD) and Alzheimer's Disease (AD). For example, in AD there is mounting evidence suggesting deficits begin with alterations of hippocampal synaptic function caused by amyloid-β (Aβ) protein prior to neuronal loss. Therefore, treatment strategies that target the initial synaptic loss, rather than late stage disease intervention, may provide a better prognosis for the treatment of AD. Furthermore, since most cognitive disorders elicit abnormalities in the form and function of dendritic spines, it would be desirable to target them directly using a small molecule to alter or alleviate these spine changes. For example, Fragile X syndrome is characterized by an overabundance of immature spines.

In some embodiments, an agent described herein, such as a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, is useful in the treatment of a mood disorder. A mood disorder is a principally psychiatric disorder in which a patient's general emotional state or mood is distorted or inconsistent with the circumstances, and interferes with the patient's ability to carry out functions of daily life. The subject may be sad, empty or irritable, or may have periods of negative feeling alternating with feelings of excessive happiness (mania).

In some embodiments, the mood disorder may be depression. Depression, sometimes referred to as major depressive disorder or clinical depression, is a common but serious mood disorder. Those who suffer from depression may experience persistent feelings of sadness and hopelessness and lose interest in activities they once enjoyed. Aside from the emotional problems caused by depression, individuals can also present with a physical symptom such as chronic pain or digestive issues. To be diagnosed with depression, symptoms generally should be present for at least two weeks. Depression may be diagnosed by a person of skill in the art, for example according to the guidelines of the Diagnostic and Statistical Manual of Mental Disorders (“DSM”). The DSM outlines the following criterion to make a diagnosis of depression. Currently, according to the DSM-5 diagnostic criteria, for a diagnosis of depression the individual must be experiencing five or more symptoms during the same 2-week period and at least one of the symptoms should be either (1) depressed mood or (2) loss of interest or pleasure. Symptoms identified in the DSM include:

1. Depressed mood most of the day, nearly every day.
2. Markedly diminished interest or pleasure in all, or almost all, activities most of the day, nearly every day.
3. Significant weight loss when not dieting or weight gain, or decrease or increase in appetite nearly every day.
4. A slowing down of thought and a reduction of physical movement (observable by others, not merely subjective feelings of restlessness or being slowed down).
5. Fatigue or loss of energy nearly every day.
6. Feelings of worthlessness or excessive or inappropriate guilt nearly every day.
7. Diminished ability to think or concentrate, or indecisiveness, nearly every day.
8. Recurrent thoughts of death, recurrent suicidal ideation without a specific plan, or a suicide attempt or a specific plan for committing suicide.

To receive a diagnosis of depression, these symptoms must cause the individual clinically significant distress or impairment in social, occupational, or other important areas of functioning. The DSM also provides for specifiers of diagnosed depression: (1) With Mixed Features—This specifier allows for the presence of manic symptoms as part of the depression diagnosis in patients who do not meet the full criteria for a manic episode; and (2) With Anxious Distress—The presence of anxiety in patients may affect prognosis, treatment options, and the patient's response to them.

Depression has many causative factors. Contributing factors to depression may include stressors such as physical abuse, psychological abuse, traumatic event(s), personal conflicts, loss of relationship with a loved one, social isolation, illness, substance abuse, or use of certain medication. A subject may have a genetic predisposition to depression. A subject may have suffered physical trauma affecting the brain such as a traumatic brain injury (TBI) or chronic traumatic encephalopathy (CTE). In some instances, depression is idiopathic. Classes of depression include major depressive disorder—prolonged and persistent periods of extreme sadness; bipolar disorder—also called manic depression or bipolar affective disorder, depression that includes alternating times of depression and mania; seasonal affective disorder (SAD)—a form of depression most often associated with fewer hours of daylight in the far northern and southern latitudes from late fall to early spring; cyclothymic disorder—a disorder that causes emotional ups and downs that are less extreme than bipolar disorder; premenstrual dysphoric disorder—mood changes and irritability that occur during the premenstrual phase of a woman's cycle and go away with the onset of menses; persistent depressive disorder (dysthymia)—long-term (chronic) but low-grade depression; disruptive mood dysregulation disorder—a disorder of chronic, severe and persistent irritability in children that often includes frequent temper outbursts that are inconsistent with the child's developmental age; depression related to medical illness—a persistent depressed mood and a significant loss of pleasure in most or all activities that's directly related to the physical effects of another medical condition; and depression induced by substance use or medication—depression symptoms that develop during or soon after substance use or withdrawal or after exposure to a medication.

In some embodiments provided is a method of treating a mood disorder in a patient in need thereof, the method comprising administering a therapeutically effective amount of a compound described herein to the patient. In some embodiments, the mood disorder is depression. In some embodiments, the patient may be refractory to treatment with an antidepressant. The antidepressant may be an antidepressant described herein.

In further embodiments, the compositions and methods are provided for alleviating, reducing, or reversing a symptom of a mood disorder. The symptom may be any symptom described herein, or known to practitioners, for example, as described in the DSM.

Symptoms of depression may include anxiety, loss of interest in daily activities; pessimism, persistent negativity; sadness, emptiness or feeling down, feelings of worthlessness, feelings of helplessness, feelings of hopelessness; fatigue, tiredness or lack of energy; low self-esteem, self-criticism and/or feeling incapable; difficulty concentrating, difficulty remembering details, and/or difficulty making decisions; persistent irritability, hostility and/or excessive anger; decreased activity, effectiveness and/or productivity; avoidance of social activities; feelings of guilt and/or worry over the past; poor appetite or overeating; suicidal ideation; low sex drive and/or loss of interest in sex; sleep disturbances, insomnia, early-morning wakefulness, or sleeping too much; restlessness, loss of interest in pleasurable activities; overeating or appetite loss, unexplained aches, unexplained pains, unexplained headache, persistent cramps, persistent digestive problems; suicidal ideation, and a suicidal act.

Provided herein are methods useful for promoting spinogenesis. In some embodiments, the method comprises administering to the subject an effective amount of an agent that binds to fascin at least at binding site 2 or binding site 3, or a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, as described herein including embodiments. Spinogenesis may be observed as an increase in the average number of spines per neuron, or a unit length of a neuron, which may be referred to as an increase in dendritic spine density. Spinogenesis may be observed as an improvement in dendritic spine morphology. For example, an improvement in dendritic spine morphology may be observed as an increase in average size of spine heads. Spinogenesis may be observed as an improvement in dendritic spine size, spine plasticity, spine motility, spine density and/or synaptic function. Spinogenesis may be observed as an increase in local spatial average of membrane potential. Spinogenesis may be observed as an increase in postsynaptic concentration (e.g., volume-averaged) of Ca²⁺. Spinogenesis may be observed as an increase in the average ratio of matured to immature spines. In some embodiments, an agent that binds to fascin at least at binding site 2 or binding site 3, or a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, increases the dendritic spine density relative to a control. In some embodiments, an agent that binds to fascin at least at binding site 2 or binding site 3, or a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, increases the dendritic spine density relative to that observed at the time that treatment is initiated. In some embodiments, the increase in dendritic spine density results in a reduction in symptoms of a neuronal disease or disorder in a subject or patient. In some embodiments, the increase in dendritic spine density is accounted for by anatomical observation. In some embodiments, the increase in dendritic spine density is observed in primary hippocampal neurons.

In some embodiments, the average dendritic spine density, relative to the time that treatment with an agent that binds to fascin at least at binding site 2 or binding site 3, or a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, is initiated, increases by at least about 5%, 10%, 15%, 20%, 25%, 30%, 35%, 40%, 45%, 50%, 60%, 70%, 80%, 90%, 100%, 125%, 150%, 175%, 200%, 250%, 300%, 400%, 500%, 750%, or 1000%, or any range between any two of the numbers, end points inclusive. In some embodiments, the dendritic spine density, relative to the time that treatment with an agent that binds to fascin at least at binding site 2 or binding site 3, or a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, is initiated, increases by about 50% to about 500%. In some embodiments, the dendritic spine density, relative to the time that treatment with an agent that binds to fascin at least at binding site 2 or binding site 3, or a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, is initiated, increases by about 100% to about 300%. In some embodiments, the dendritic spine density, relative to the time that treatment with an agent that binds to fascin at least at binding site 2 or binding site 3, or a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, is initiated, increases by about 200% to about 300%. In some embodiments, the duration of treatment with an agent that binds to fascin at least at binding site 2 or binding site 3, or a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, is 15 minutes, 30 minutes, 1 hour, 2 hours, 4 hours, 8 hours, 1 day, 3 days, 5 days, 7 days, 14 days, 28 days, 90 days, 180 days, or 365 days.

In some embodiments, the method increases spine density through promoting the formation of new spines. In some embodiments, the method increases the average spine density by at least about 5%, 10%, 15%, 20%, 25%, 30%, 35%, 40%, 45%, 50%, 60%, 70%, 80%, 90%, 100%, 125%, 150%, 175%, 200%, 250%, 300%, 400%, 500%, 750%, or 1000%, or any range between any two of the numbers, end points inclusive, relative to a control (e.g., the spine density in the absence of the compound). In some embodiments, the method increases the average spine density about 50% to about 500% relative to a control (e.g., the spine density in the absence of the compound). In some embodiments, the method increases the spine density about 100% to about 300% relative to a control (e.g., the spine density in the absence of the compound). In some embodiments, the method increases the spine density about 200% to about 300% relative to a control (e.g., the spine density in the absence of the compound).

In some embodiments, the method increases spine density through increasing a neuron length. In some embodiments, the method increases the average neuron length, relative to the time that treatment with an agent that binds to fascin at least at binding site 2 or binding site 3, or a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, is initiated, by about 100 nm, 300 nm, 500 nm, 700 nm, 1 micron, 2 microns, 3 microns, 4 microns, 5 microns, 7 microns, 10 microns, 15 microns, 20 microns, 25 microns, or any range between any two of the numbers, end points inclusive. In some embodiments, the method increases the average neuron length about 500 nm to about 25 microns relative to a control (e.g., the neuron length in the absence of the compound). In some embodiments, the method increases the neuron length about 10% to about 300% relative to a control (e.g., the neuron length in the absence of the compound). In some embodiments, the method increases the neuron length about 200% to about 300% relative to a control (e.g., the neuron length in the absence of the compound).

In some embodiments, the method increases the average number of spines per neuron, relative to the time that treatment with an agent that binds to fascin at least at binding site 2 or binding site 3, or a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, is initiated. In some embodiments, average number spines per unit length of a neuron increases by at least about 10, 20, 30, 40, 50, 60, 70, 80, 90, 100, 200, 300, 400, 500, 600, 700, 800, 900, or about 1000 more, or any range between any two of the numbers, end points inclusive. In some embodiments, the time is 1 hour, 2 hours, 4 hours, 8 hours, 1 day, 3 days, 5 days, 7 days, 14 days, 28 days, 90 days, 180 days, or 365 days.

In some embodiments, the compounds are useful in the treatment of neuronal diseases and disorders. A neuronal disease is a disease or condition in which the function of a subject's nervous system becomes impaired. The neuronal disease or disorder may be a neurological disease or disorder. The neuronal disease or disorder may be associated with a neurodegenerative disease or disorder.

In an aspect is provided a method of treating a neuronal disease in a patient in need thereof, the method comprising administering a therapeutically effective amount of an agent that binds to fascin at least at binding site 2 or binding site 3, or a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, to the patient. In some embodiments, the neuronal disease is Alzheimer's disease. In some embodiments, the neuronal disease is Parkinson's disease. In some embodiments, the neuronal disease is Parkinson's dementia. In some embodiments, the neuronal disease is autism. In some embodiments, the neuronal disease is fragile X syndrome.

In some embodiments, the disease or disorder is related to (e.g. characterized by) an accumulation of amyloid plaques. In some embodiments, the neuronal disease is a traumatic brain injury. In some embodiments, a patient having a neuronal disease has suffered a traumatic brain injury before, during, or after the onset of the neuronal disease. In some embodiments, the neuronal disease includes a neuronal impairment. A neuronal impairment may include atrophy or other decrease in the effective functioning of the neuron. For example, it is known that Alzheimer's disease presents with neuronal impairment, especially in cortical neurons, e.g., hippocampal neurons and neurons in proximity to the hippocampus. Loss of synapses may correlate with loss of dendritic spines and neurodegeneration.

In some embodiments, the neuronal disease is associated with abnormal dendritic spine morphology, spine size, spine plasticity, spine motility, spine density and/or abnormal synaptic function. In some embodiments, the neuronal disease is associated with an abnormal (e.g., reduced) level of dendritic spine density.

In some embodiments, the neuronal disease is Alzheimer's disease. In some embodiments, the neuronal disease is Parkinson's disease. In some embodiments, the neuronal disease is Parkinson's disease accompanied by dementia. In some embodiments, the neuronal disease is autism. In some embodiments, the neuronal disease is stroke. In some embodiments, the neuronal disease is posttraumatic stress disorder (PTSD). In some embodiments, the neuronal disease is traumatic brain disorder (TBD). In some embodiments, the neuronal disease is chronic traumatic encephalopathy (CTE). In some embodiments, the neuronal disease is schizophrenia. In some embodiments, the neuronal disease is dementia (e.g., general dementia). In some embodiments, the neuronal disease is attention-deficit/hyperactivity disorder (ADHD). In some embodiments, the neuronal disease is amyotrophic lateral sclerosis (ALS). In some embodiments, the neuronal disease is frontotemporal lobar degeneration (FTLD) (e.g., FTLD-tau, FTLD-TDP, or FTLD-FUS). In some embodiments, the neuronal disease is memory loss. In some embodiments, the neuronal disease includes memory loss. In some embodiments, the neuronal disease is age-related memory loss. In some embodiments, the neuronal disease includes age-related memory loss. In some embodiments, the neuronal disease is hypertensive encephalopathy. In some embodiments, the neuronal disease is chronic stress. In some embodiments, the neuronal disease includes chronic stress. In some embodiments, the neuronal disease is FTLD-TDP Type A. In some embodiments, the neuronal disease is FTLD-TDP Type B. In some embodiments, the neuronal disease is FTLD-TDP Type C. In some embodiments, the neuronal disease is FTLD-TDP Type D.

Examples of neuronal diseases that may be treated with a compound or method described herein include Alexander's disease, Alper's disease, Alzheimer's disease, depression, perinatal asphyxia, Parkinson's disease dementia (“PD dementia”), amyotrophic lateral sclerosis, ataxia telangiectasia, Batten disease (also known as Spielmeyer-Vogt-Sjogren-Batten disease), spongiform encephalopathy (e.g., bovine spongiform encephalopathy (mad cow disease), Kuru, Creutzfeldt-Jakob disease, fatal familial insomnia, Canavan disease, Cockayne syndrome, corticobasal degeneration, fragile X syndrome, frontotemporal dementia, Gerstmann-Straussler-Scheinker syndrome, Huntington's disease, HIV-associated dementia, Kennedy's disease, Krabbe's disease, Lewy body dementia, Machado-Joseph disease (Spinocerebellar ataxia type 3), multiple sclerosis, multiple system atrophy, narcolepsy, neuroborreliosis, Parkinson's disease, Pelizaeus-Merzbacher Disease, Pick's disease, primary lateral sclerosis, prion diseases, Refsum's disease, Sandhoff s disease, Schilder's disease, subacute combined degeneration of spinal cord secondary to pernicious anaemia, schizophrenia, spinocerebellar ataxia (multiple types with varying characteristics), spinal muscular atrophy, Steele-Richardson-Olszewski disease, Tabes dorsalis, drug-induced Parkinsonism, progressive supranuclear palsy, corticobasal degeneration, multiple system atrophy, idiopathic Parkinson's disease, autosomal dominant Parkinson disease, familial, type 1 (PARK1), Parkinson disease 3, autosomal dominant Lewy body (PARK3), Parkinson disease 4, autosomal dominant Lewy body (PARK4), Parkinson disease 5 (PARK5), Parkinson disease 6, autosomal recessive early-onset (PARK6), Parkinson disease 2, autosomal recessive juvenile (PARK2), Parkinson disease 7, autosomal recessive early-onset (PARK7), Parkinson disease 8 (PARK8), Parkinson disease 9 (PARK9), Parkinson disease 10 (PARK10), Parkinson disease 11 (PARK11), Parkinson disease 12 (PARK12), Parkinson disease 13 (PARK13), or mitochondrial Parkinson's disease. In some embodiments, the neuronal disease is Alzheimer's disease, Parkinson's disease, Parkinson's dementia, autism, stroke, post-traumatic stress disorder (PTSD), traumatic brain disorder (TBD), chronic traumatic encephalopathy (CTE), schizophrenia, dementia (e.g., general dementia), attention-deficit/hyperactivity disorder (ADHD), amyotrophic lateral sclerosis (ALS), frontotemporal lobar degeneration (FTLD) (e.g., FTLD-tau, FTLD-TDP, or FTLD-FUS), memory loss (e.g., age-related memory loss), hypertensive encephalopathy, or chronic stress.

In some embodiments, the neuronal disease is Alzheimer's disease (AD). Alzheimer's disease is characterized by symptoms of memory loss in the early stages of the disease. Apoε4 carriers are at greater risk of developing AD. APOε4 is believed to be less efficient than other isoforms at clearing Aε, and thus may be correlated with greater amyloid burden, tau phosphorylation, synaptotoxicity, and reduced synaptic density. Having experienced a traumatic brain injury (TBI) is another risk factor for developing AD, and studies indicate that those who experience a TBI have a significantly increased risk of AD. Cognitive decline has been correlated with the progressive loss of synapses. As the disease advances, symptoms include confusion, long-term memory loss, paraphasia, loss of vocabulary, aggression, irritability and/or mood swings. In more advanced stages of the disease, there is loss of bodily functions. Patients with Alzheimer's Disease (AD) demonstrate many characteristic neuropathies such as increased oxidative stress, mitochondrial dysfunction, synaptic dysfunction, disruption of calcium homeostasis, deposition of senile plaques and neurofibrillary tangles, and atrophy of the brain. Without wishing to be bound by any theory, it is believed that both the cause and effect of these neuropathies is the accumulation of harmful forms of aggregated amyloid beta (Aβ) peptides in the brain. AD related disorders include senile dementia of AD type (SDAT), frontotemporal dementia (FTD), vascular dementia, mild cognitive impairment (MCI) and age-associated memory impairment (AAMI). In some embodiments, a method of treating or preventing Alzheimer's disease is provided, comprising administering to a patient in need thereof a therapeutically effective amount of an agent that binds to fascin at least at binding site 2 or binding site 3, or a compound described herein, such as compound 1. In some embodiments, the patient is an Apoε2 or Apoε3 carrier. In some embodiments, the patient has suffered a TBI. In some embodiments, the patient is an Apoε4 carrier. In some embodiments, the patient is an Apoε4 carrier who has suffered a TBI.

In some embodiments the neuronal disease is Fragile-X syndrome (FXS). As known in the art, FXS is a genetic syndrome which has been linked to a variety of disorders (e.g., autism and inherited intellectual disability). The disability can present in a spectrum of values ranging from mild to severe. It is observed that males with FXS begin developing progressively more severe problems, typically starting after age 40, in performing tasks which require working memory. This is especially observed with respect to verbal working memory. In some embodiments, the neuronal disease is autism. As known in the art, autism is a disorder of neural development. Without wishing to be bound by any theory, it is believed that autism affects information processing in the brain by altering how nerves and synapses connect and organize.

In further embodiments, the compositions and methods are provided for alleviating, reducing, or reversing a symptom of a neuronal disease or disorder. The symptom may be any symptom described herein.

The term “memory” and the like refer, in the usual and customary sense, to the processes by which information is encoded, stored and retrieved by a subject. The terms “encode,” “register” and the like in the context of memory refer, in the usual and customary sense, to receiving, processing and combining information impinging on the senses as chemical or physical stimuli. The term “stored” and the like in this context refer, in the usual and customary sense, to the creation of a record of the encoded information. The terms “retrieve,” “recall” and the like in this context refer, in the usual and customary sense, to calling back the stored information. Retrieval can be in response to a cue, as known in the art. In some embodiments, memory loss refers to a diminished ability to encode, store, or retrieve information. In some embodiments, the memory may be recognition memory or recall memory. In this context, “recognition memory” refers to recollection of a previously encountered stimulus. The stimulus can be e.g., a word, a scene, a sound, a smell or the like, as known in the art. A broader class of memory is “recall memory” which entails retrieval of previously learned information, e.g., a series of actions, list of words or number, or the like, which a subject has encountered previously. Methods for assessing the level of memory encoding, storage and retrieval demonstrated by a subject are well known in the art, including methods disclosed herein. For example, in some embodiments the method improves memory in a subject in need thereof, wherein the subject has a neuronal disease. In some embodiments, the method improves memory in the subject. In some embodiments, the method treats neuronal or cognitive impairment in the subject. In some embodiments, the method treats neuronal impairment in the subject. In some embodiments, the method treats cognitive impairment in the subject.

Further to any aspect disclosed herein, in some embodiments the subject suffers from brain injury. Types of brain injury include brain damage (i.e., destruction or degeneration of brain cells), traumatic brain injury (i.e., damage accruing as the result of an external force to the brain), stroke (i.e., a vascular incident which temporarily or permanently damages the brain, e.g., via anoxia), and acquired brain injury (i.e., brain damage not present at birth). In some embodiments, the method improves memory in the subject. In some embodiments, the method improves learning in the subject. In some embodiments, the method treats neuronal or cognitive impairment in the subject. In some embodiments, the method treats neuronal impairment in the subject. In some embodiments, the method treats cognitive impairment in the subject.

In some embodiments, a method for promoting spinogenesis in a patient in need thereof is provided, comprising administering to the patient a compound that inhibits fascin. In some embodiments, a method of treating or preventing a neuronal disease or disorder is provided, comprising administering to a patient in need thereof a therapeutically effective amount of an agent that binds to fascin at least at binding site 2 or binding site 3, or a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof. In some embodiments, a compound for use in the treatment of a neuronal disease or disorder is provided, wherein the compound is a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof. In some embodiments, a compound for use in the manufacture of a medicament for the treatment of a neuronal disease or disorder is provided, wherein the compound is a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof. In some embodiments, the neuronal disease or disorder is selected from Alzheimer's disease, Parkinson's disease, Parkinson's dementia, autism, fragile X syndrome, and traumatic brain injury. In some embodiments, the neuronal disease or disorder is Alzheimer's disease. In some embodiments, an agent that binds to fascin at least at binding site 2 or binding site 3, or a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, inhibits cross-linking of f-actin. In some embodiments, an agent that binds to fascin at least at binding site 2 or binding site 3, or a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, is anti-metastatic.

### Combination Therapies

In one embodiment, the compounds disclosed herein may be used in combination with one or more additional therapeutic agent that are being used and/or developed to treat a neuronal disease or disorder.

When used for the treatment or prevention of the diseases and disorders described above, an agent that binds to fascin at least at binding site 2 or binding site 3, or a compound of formula I, formula II, formula IV, formula V, formula VII, formula VIII, formula IX, formula X, or formula XI as provided herein, or a pharmaceutically acceptable salt thereof, or compound 1, compound 8, compound 9, compound 10, or compound 11 or a pharmaceutically acceptable salt thereof, may be administered together with one or more additional therapeutic agents, for example additional therapeutic agents approved for use in the treatment or prevention of the particular disease or disorder, and more particularly agents considered to form the current standard of care. Where combination therapy is envisaged, the active agents may be administered simultaneously, separately or sequentially in one or more pharmaceutical compositions.

Recent strategies for the treatment of AD, therefore, include controlling the production or the aggregation state of specific isoforms of Aβ peptides. Additional strategies include preventing, reducing or removing toxic forms of phosphorylated tau. Other strategies involve small molecule targeting of enzymes that play a role in production of Aβ peptides through processing of amyloid precursor protein in an attempt to lower the abundance of Aβ peptides in the brain. Additionally, there is accruing information on the role of non-amyloid neuropathies such as tauopathy or sporadic inheritance of specific mutations in the apolipoprotein E gene, which is stimulating additional strategies to combat neurodegeneration.

The one or more additional therapeutic agent may be tacrine, donepezil, galantamine, rivastigmine, memantine, levodopa, carbidopa, lisuride, rasagiline, tolcapone, entacapone, clozapine, desipramine, citalopram, nortriptyline, paroxetine, atomoxetine, venlafaxine, amantadine, donepezil, rivastigmine, bromocriptine, cabergoline, pergolide, pramipexole, ropinirole, rotigotine, apomorphine, benserazide, selegiline, omigapil, CEP-1347, isradipine, DOPA, lithium, riluzole, levetiracetam, ezogabine, pregabalin, rufmamide, felbamate, carbamazepine, valproate, sodium valproate, lamotrigine, phenytoin, oxcarbazepine, ethosuximide, gabapentin, tiagabine, topiramate, vigabatrin, phenobarbital, primidone, clonazepam, interferon beta-la, interferon beta-lb, mitoxantrone, natalizumab, fmgolimod, natalizumab, teriflunomide, dimethyl fumarate, glatiramer, ATOH1 gene therapy, ozanezumab, arimoclomol, tirasemtiv, dexpramipexole, pridopidine, or galantamine; or a phosphoglycerate kinase (PGK) as described in US 2018/0147263. In some embodiments, the one or more additional therapeutic agent may be an acetyl-cholinesterase inhibitor (AChEI), for example, acotiamide, alpha-pinene, ambenonium, demecarium, DFP (diisopropylfluorophosphate), donepezil, edrophonium, galantamine, huperzine A, lactucopicrin, ladostigil, neostigmine, physostigmine, pyridostigmine, dyflos, echothiophate, rivastigmine, rosmarinic acid, tacrine, ungeremine, zanapezil, ganstigmine, phenserine, phenethylnorcymserine (PENC), cymserine, thiacymserine, SPH 1371 (galantamine plus), ER 127528, RS 1259, or F3796. In some embodiments, the one or more additional therapeutic agent may be an amyloid-clearing antibody, for example, bapineuzumab, solanezumab, gantenerumab, crenezumab, ponezumab, BAN2401, or aducanumab.

The one or more additional therapeutic agents may be a sedative-hypnotic such as chloral hydrate, estazolam, flurazepam hydrochloride, pentobarbital, pentobarbital sodium, phenobarbital sodium, secobarbital sodium, temazepam, triazolam, zaleplon, or zolpidem tartrate; an anticonvulsant such as acetazolamide sodium, carbamazepine, clonazepam, clorazepate dipotassium, diazepam, divalproex sodium, ethosuximde, fosphenytoin sodium, gabapentin, lamotrigine, magnesium sulfate, phenobarbital, phenobarbital sodium, phenytoin, phenytoin sodium, primidone, tiagabine hydrochloride, topiramate, valproate sodium, or valproic acid; an antidepressant such as amitriptyline hydrochloride, amitriptyline pamoate, amoxapine, bupropion hydrochloride, citalopram hydrobromide, clomipramine hydrochloride, desipramine hydrochloride, doxepin hydrochloride, fluoxetine hydrochloride, imipramine hydrochloride, imipramine pamoate, mirtazapine, nefazodone hydrochloride, nortriptyline hydrochloride, paroxetine hydrochloride, phenelzine sulfate, sertraline hydrochloride, tranylcypromine sulfate, trimipramine maleate, or venlafaxine hydrochloride; an antianxiety drug such as alprazolam, buspirone hydrochloride, chlordiazepoxide, chlordiazepoxide hydrochloride, clorazepate dipotassium, diazepam, doxepin hydrochloride, hydroxyzine embonate, hydroxyzine hydrochloride, hydroxyzine pamoate, lorazepam, mephrobamate, midazolam hydrochloride, or oxazepam; an antipsychotic drug such as chlorpromazine hydrochloride, clozapine, fluphenazine decanoate, fluephenazine enanthate, fluphenazine hydrochloride, haloperidol, haloperidol decanoate, haloperidol lactate, loxapine hydrochloride, loxapine succinate, mesoridazine besylate, molindone hydrochloride, olanzapine, perphenazine, pimozide, prochlorperazine, quetiapine fumarate, risperidone, thioridazine hydrochloride, thiothixene, thiothixene hydrochloride, or trifluoperazine hydrochloride; a central nervous system stimulant such as amphetamine sulfate, caffeine, dextroamphetamine sulfate, doxapram hydrochloride, methamphetamine hydrochloride, methylphenidate hydrochloride, modafinil, pemoline, or phentermine hydrochloride; an antiparkinsonian such as amantadine hydrochloride, benztropine mesylate, biperiden hydrochloride, biperiden lactate, bromocriptine mesylate, carbidopa-levodopa, entacapone, levodopa, pergolide mesylate, pramipexole dihydrochloride, ropinirole hydrochloride, selegiline hydrochloride, tolcapone, or trihexyphenidyl hydrochloride; or a central nervous system agent such as bupropion hydrochloride, donepezil hydrochloride, droperidol, fluvoxamine maleate, lithium carbonate, lithium citrate, naratriptan hydrochloride, nicotine polacrilex, nicotine transdermal system, propofol, rizatriptan benzoate, sibutramine hydrochloride monohydrate, sumatriptan succinate, tacrine hydrochloride, or zolmitriptan; a cholinergic (e.g., parasymathomimetic) such as bethanechol chloride, edrophonium chloride, neostigmine bromide, neostigmine methyl sulfate, physostigmine salicylate, or pyridostigmine bromide; an anticholinergic such as atropine sulfate, dicyclomine hydrochloride, glycopyrrolate, hyoscyamine, hyoscyamine sulfate, propantheline bromide, scopolamine, scopolamine butylbromide, or scopolamine hydrobromide; an adrenergic (sympathomimetics) such as dobutamine hydrochloride, dopamine hydrochloride, metaraminol bitartrate, norepinephrine bitartrate, phenylephrine hydrochloride, pseudoephedrine hydrochloride, or pseudoephedrine sulfate; an adrenergic blocker (sympatholytic) such as dihydroergotamine mesylate, ergotamine tartrate, methysergide maleate, or propranolol hydrochloride; a skeletal muscle relaxant such as baclofen, carisoprodol, chlorzoxazone, cyclobenzaprine hydrochloride, dantrolene sodium, methocarbamol, or tizanidine hydrochloride; a neuromuscular blocker such as atracurium besylate, cisatracurium besylate, doxacurium chloride, mivacurium chloride, pancuronium bromide, pipecuronium bromide, rapacuronium bromide, rocuronium bromide, succinylcholine chloride, tubocurarine chloride, or vecuronium bromide; or a corticosteroid such as betamethasone, betamethasone acetate or betamethasone sodium phosphate, betamethasone sodium phosphate, cortisone acetate, dexamethasone, dexamethasone acetate, dexamethasone sodium phosphate, fludrocortisone acetate, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, prednisolone, prednisolone acetate, prednisolone sodium phosphate, prednisolone tebutate, prednisone, triamcinolone, triamcinolone acetonide, or triamcinolone diacetate.

The one or more additional therapeutic agents may be an antidepressant selected from levomilnacipran, venlafaxine, desvenlafaxine, sibutramine, nefazodone, milnacipran, duloxetine, bicifadine, tesofensine, brasofensine, isocarboxazid, moclobemide, phenelzine, tranylcypromine, selegiline, rasagiline, nialamide, iproniazid, iproclozide, toloxatone, butriptyline, amoxapine, amitriptyline, nortriptyline, clomipramine, desipramine, dosulepin, doxepin, imipramine, dibenzepin, iprindole, lofepramine, opipramol, protriptyline, trimipramine, fluoxetine, norfluoxetine, citalopram, dapoxetine, escitalopram, fluvoxamine, paroxetine, sertraline, ketamine, esketamine, bupropion, mirtazapine, vilazodone, vortioxetine, aripiprazole, and St. John's Wort.

### Kits

Provided herein are also kits that include compounds described herein, or a pharmaceutically acceptable salt thereof, optionally a second active ingredient, and suitable packaging. In one embodiment, a kit further includes instructions for use. In one aspect, a kit includes a compound, or a pharmaceutically acceptable salt thereof, and a label and/or instructions for use of the pharmaceutical composition in the treatment of the indications, including the diseases or conditions, described herein.

Provided herein are also articles of manufacture that include a compound described herein, or a pharmaceutically acceptable salt thereof in a suitable container. The container may be a vial, jar, ampoule, preloaded syringe, nebulizer, aerosol dispensing device, dropper, or intravenous bag.

### Pharmaceutical Compositions and Modes of Administration

Compounds provided herein are usually administered in the form of pharmaceutical compositions. Thus, provided herein are also pharmaceutical compositions that contain one or more of the compounds described herein, including generally a compound described herein, or a pharmaceutically acceptable salt thereof and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain some embodiments, the pharmaceutical composition may be administered by intra-arterial injection, intravenously, intraperitoneally (“i p”), parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compositions described herein. Administration may be via, for example, capsule or enteric coated tablets. In making the pharmaceutical compositions that include at least one compound described herein or a pharmaceutically acceptable salt thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The pharmaceutical composition and any container in which it is distributed may be sterilized. The pharmaceutical composition may also contain adjuvants such as preservatives, stabilizers, emulsifiers or suspending agents, wetting agents, salts for varying the osmotic pressure, viscosity alerting agents, or buffers.

The compositions that include at least one compound described herein, such as a compound described herein, or a pharmaceutically acceptable salt thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The pharmaceutical composition may be formulated for nasal administration. Such pharmaceutical compositions may include one or more active ingredients, such as a compound described herein, or a pharmaceutically acceptable salt thereof, in varying physical states. For example, the active ingredients may be dissolved or suspended in a liquid carrier. The active ingredients may be in a dry form. The dry form may be a powder. Active ingredients in a powder may be amorphous or crystalline. For example, a compound described herein, or a pharmaceutically acceptable salt thereof, may be amorphous or crystalline. The crystalline active material may be a hydrate or a solvate.

Solid compounds, or a salt or crystal thereof, may be present in a formulation in a selected average particle size. The particles may have an average particle size (in longest dimension) of 10 nm, 100 nm, 300 nm, 500 nm, 1 μm, 10 μm, 50 μm, 100 μm, 300 μm, or 500 μm, or a range between any two values.

Administration may be by inhalation or insufflation. Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route. Effects may be local or systemic. In a particular embodiment, the effect is local to cranial tissues. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner. A pharmaceutical composition for inhalation or insufflation may be an aerosol.

The pharmaceutical composition may comprise a liquid suspension or solution comprising about 0.05%, about 0.1%, about 0.3%, about 0.5%, about 0.7%, about 1%, about 2%, about 3%, about 4%, or about 5% w/w of active ingredients. The liquid may comprise water and/or an alcohol. The liquid may include a pH adjusting agent such that the pH is about 1, about 2, about 3, about 4, about 5, about 6, about 7, about 8, about 9, or about 10, or a range of values therebetween.

The pharmaceutical composition may comprise a pharmaceutically acceptable preservative. Preservatives suitable for use herein include, but are not limited to, those that protect the solution from contamination with pathogenic particles, including phenylethyl alcohol, benzalkonium chloride, benzoic acid, or benzoates such as sodium benzoate. In certain some embodiments, the pharmaceutical composition comprises from about 0.01% to about 1.0% w/w of benzalkonium chloride, or from about 0.01% and about 1% v/w phenylethyl alcohol. Preserving agents may also be present in an amount from about 0.01% to about 1%, preferably about 0.002% to about 0.02% by total weight or volume of the composition.

The pharmaceutical composition may also comprise from about 0.01% to about 90%, or about 0.01% to about 50%, or about 0.01% to about 25%, or about 0.01% to about 10%, or about 0.01% to about 1% w/w of one or more of an emulsifying agent, a wetting agent or a suspending agent. Such agents for use herein include, but are not limited to, polyoxyethylene sorbitan fatty esters or polysorbates, including, but not limited to, polyethylene sorbitan monooleate (Polysorbate 80), polysorbate 20 (polyoxyethylene (20) sorbitan monolaurate), polysorbate 65 (polyoxyethylene (20) sorbitan tristearate), polyoxyethylene (20) sorbitan mono-oleate, polyoxyethylene (20) sorbitan monopalmitate, polyoxyethylene (20) sorbitan monostearate; lecithins; alginic acid; sodium alginate; potassium alginate; ammonium alginate; calcium alginate; propane-1,2-diol alginate; agar; carrageenan; locust bean gum; guar gum; tragacanth; acacia; xanthan gum; karaya gum; pectin; amidated pectin; ammonium phosphatides; microcrystalline cellulose; methyl cellulose; hydroxypropylcellulose; hydroxypropylmethylcellulose; ethylmethylcellulose; carboxymethylcellulose; sodium, potassium and calcium salts of fatty acids; mono- and di-glycerides of fatty acids; acetic acid esters of mono- and di-glycerides of fatty acids; lactic acid esters of mono- and di-glycerides of fatty acids; citric acid esters of mono- and di-glycerides of fatty acids; tartaric acid esters of mono- and di-glycerides of fatty acids; mono- and diacetyltartaric acid esters of mono- and di-glycerides of fatty acids; mixed acetic and tartaric acid esters of mono- and di-glycerides of fatty acids; sucrose esters of fatty acids; sucroglycerides, polyglycerol esters of fatty acids; polyglycerol esters of polycondensed fatty acids of castor oil; propane-1,2-diol esters of fatty acids; sodium stearoyl-2lactylate; calcium stearoyl-2-lactylate; stearoyl tartrate; sorbitan monostearate; sorbitan tristearate; sorbitan monolaurate; sorbitan monooleate; sorbitan monopalmitate; extract of quillaia; polyglycerol esters of dimerised fatty acids of soya bean oil; oxidatively polymerised soya bean oil; and pectin extract.

In a further embodiment, the pharmaceutical composition for nasal administration may be provided in the form of a powder. For example, a powdery nasal composition can be directly used as a powder for a unit dosage form. If desired, the powder can be filled in capsules such as hard gelatine capsules. The contents of the capsule or single dose device may be administered using e.g. an insufflator.

Thus, a method for treating a neuronal disorder may include the step of administering nasally a pharmaceutical composition comprising a compound described herein, or a salt thereof, to a subject in need thereof.

### Dosing

The specific dose level of an active ingredient of the present application, for example a compound described herein, of a salt thereof, for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of a compound described herein, or a salt thereof, may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

When administered nasally, the total daily dosage for a human subject may be between 1 mg and 1,000 mg, between about 1,000-2,000 mg/day, between about 10-500 mg/day, between about 50-300 mg/day, between about 75-200 mg/day, or between about 100-150 mg/day. In various embodiments, the daily dosage is about 10 mg, about 30 mg, about 50 mg, about 75 mg, about 100 mg, about 200 mg, about 300 mg, about 400 mg, about 500 mg, about 600 mg, about 700 mg, about 800 mg, about 900 mg, or about 1000 mg, or a range of values therebetween.

The active ingredients of the present application or the pharmaceutical compositions thereof may be administered once, twice, three, or four times daily, using any suitable mode described above. Also, administration or treatment may be continued for a number of days; for example, commonly treatment would continue for at least 7 days, 14 days, or 28 days, for one cycle of treatment. Treatment cycles are well known, and are frequently alternated with resting periods of about 1 to 28 days, commonly about 7 days or about 14 days, between cycles. The treatment cycles, in other embodiments, may also be continuous. Administration or treatment may be continued indefinitely.

In a particular embodiment, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

### EXAMPLES

### Example 1

### Analysis and Preparation of Fascin Crystal Structures

All available Fascin crystal structures were downloaded from the PDB and prepared for structure analysis. The structures were analyzed by eye and by standard automated protocols embedded in MolSoft's ICM-Pro software. Flydrogen atoms were added to the structures, and considerations were made regarding: correct orientation of Asn and Gin side-chains, ligand and protein charges, histidines orientation and protonation state and any crystallographic quality flags such as high b-factors or low occupancy.

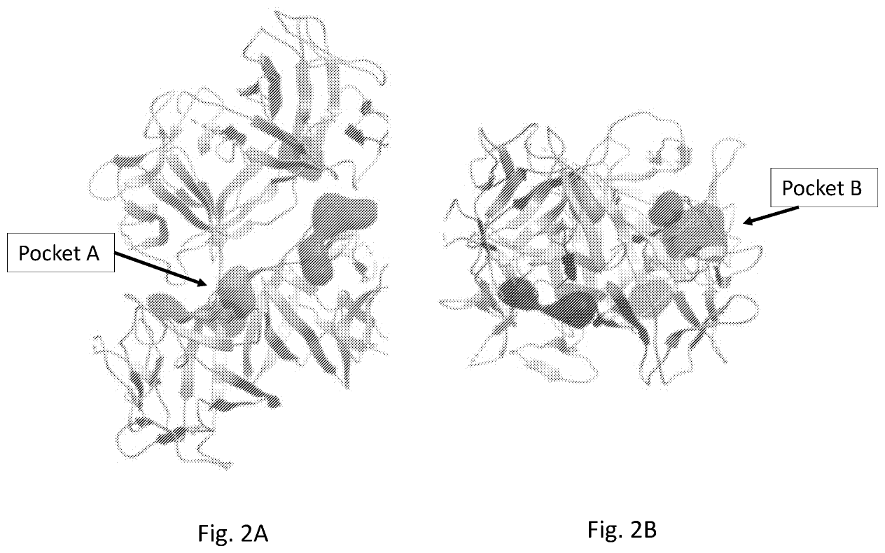

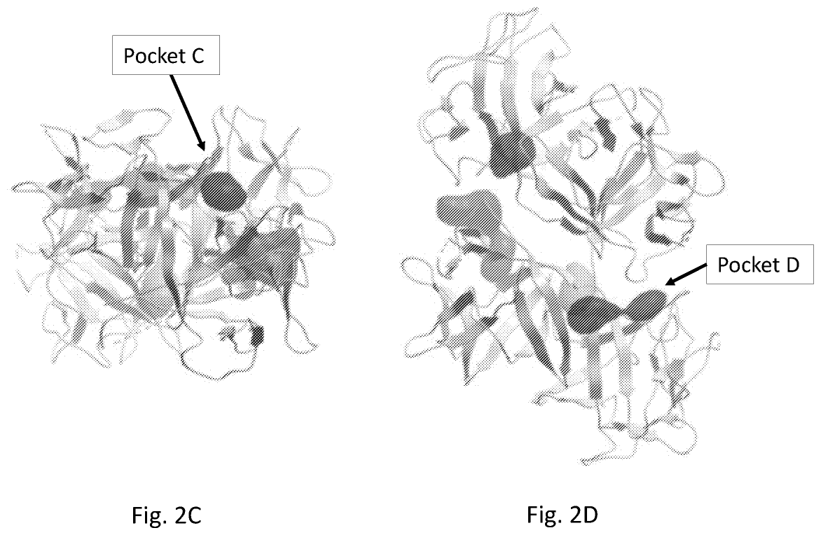

MolSoft's ICMPocketFinder algorithm was used to identify potential ligand binding pockets and cavities in all the available fascin crystal structures. Pockets were searched for in the active chain A of crystal structure 3LLP, this structure has the highest resolution (1.8 Å). FIGS. 2A-2D provide “front,” “bottom,” “top,” and “back” views of fascin, respectively, in which pockets A-D are indicated.

Four “drug-like” pockets (pockets A-D) were identified which are believed to have properties suitable for binding small molecules.

### Ligand Docking and Scoring

The head groups and head+tail of comparative compound 2, comparative compound 3, and comparative compound 4 were docked to each of the four pockets shown in FIG. 1 using MolSoft's ICM-Docking software (Version 3.8-6a) 7. The docking scores to each of the pockets are shown in Table 1—the lower the docking score the better the ligand interaction is.

Comparative compound 2 has the structure:

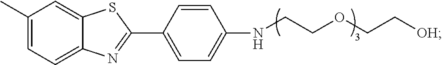

comparative compound 3 has the structure:

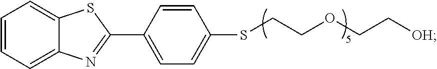

comparative compound 4 has the structure:

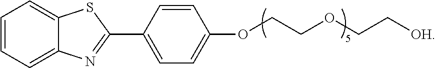

TABLE 1
Docking scores for the ligands to the four pockets A, B,
C, and D colored as shown in FIG. 1. In all cases except
one, ligands bound with a better score to pocket B.

| | Head | | | Head + Tail | | |
|---|---|---|---|---|---|---|
| | comp. | comp. | comp. | comp. | comp. | comp. |
| | com- | com- | com- | com- | com- | com- |
| Pocket | pound 2 | pound 3 | pound 4 | pound 2 | pound 3 | pound 4 |
| A | −21 | −20 | −19 | −17 | −25 | −14 |
| B | −23 | −24 | −25 | −29 | −39 | −44 |
| C | −19 | −19 | −14 | −18 | −22 | −18 |
| D | −25 | −19 | −24 | −24 | −12 | −27 |

Pocket B which is located at the Actin Binding Site 1, as seen in FIG. 1, resulted in the lowest docking score. This site was further investigated in the other fascin crystal structures and this pocket is close to the pentaethylene glycol binding site in PDB 3P53. The head group was docked to Pocket B in PDB 3P53 and significantly better scores were achieved with the head group.

TABLE 2
Results of docking the Head group of comparative compound
2, comparative compound 3 and comparative compound 4 to Pocket
B in PDB 3P53 which contains a pentaethylene glycol.

| | Head | | |
|---|---|---|---|
| | comparative | comparative | comparative |
| Pocket | compound 2 | compound 3 | compound 4 |
| B | −54 | −57 | −57 |

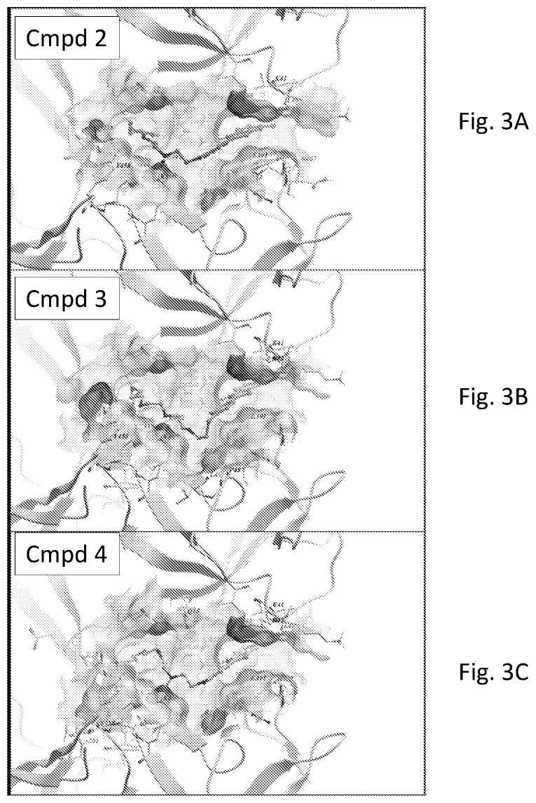

The tails were then docked using the docked head groups as an anchor point. The final energetically favorable ligand poses are shown in (FIG. 3A-3C).

FIGS. 3A-3C depict docked complexes of human fascin 1 and comparative compound 2, comparative compound 3, and comparative compound 4. All three ligands make a hydrogen bond from the nitrogen in the benzothiazole ring to ARG 389 and the first ethylene glycol makes hydrogen bond with LYS 460.

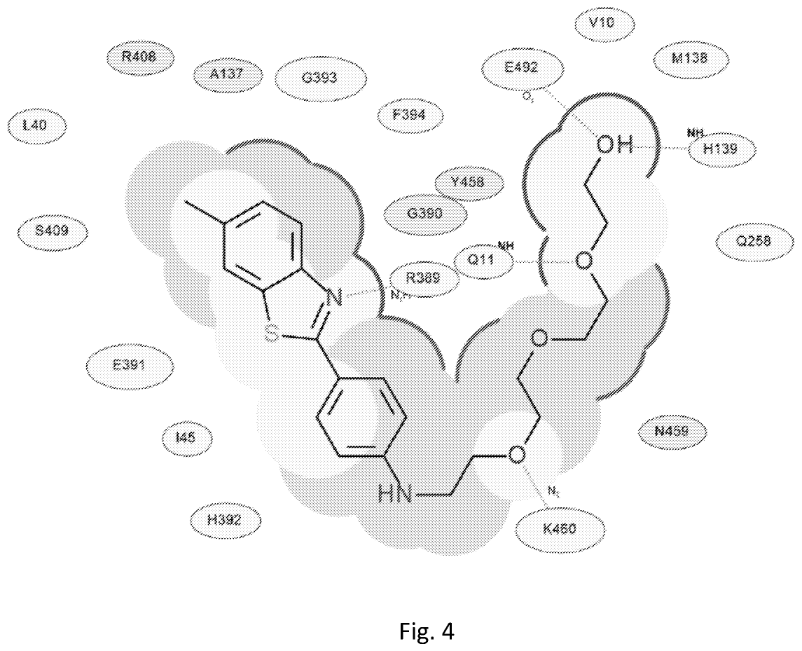

FIG. 4 depicts a 2D interaction diagram of the comparative compound 2 and human fascin 1 complex. Dashed arrows represent hydrogen bonds. The thick line around the ligand shape indicates accessible surface. Size of residue ellipse represents the strength of the contact. 2D distance between residue label and ligand represents proximity.

### Example 2

A crystal structure of fascin bound to compound 1 (Protein Data Bank (PDB) 6B0T) was prepared for structure analysis, as depicted in FIG. 5. The structure was analyzed by eye and by standard automated protocols embedded in MolSoft's ICM-Pro software. Hydrogen atoms were added to the structures, and considerations were made regarding: correct orientation of Asn and Gin side-chains, ligand and protein charges, histidines orientation and protonation state and any crystallographic quality flags such as high b-factors or low occupancy. MolSoft's ICMPocketFinder algorithm was used to define the binding pocket for docking. Fascin binding sites were described by nearest neighbor residues to a ligand (residues within a distance of 3 Å). With reference to FIG. 5, fascin binding site 1 was defined by V10, Q11, L40, K41, A137, H139, Q141, Q258, S259, R383, R389, E391, G393, F394, S409, Y458, K460, E492, and Y493, while binding site 2 was defined by F14, L16, L48, Q50, L62, W101, L103, E215, and S218.

### Ligand Docking and Scoring

Comparative compound 2, comparative compound 3, and comparative compound 4 were docked to each binding site to determine the most favorable binding site for each compound. The predicted energetically favorable binding conformation of comparative compound 2, comparative compound 3, and comparative compound 4 are reported above.

The head groups and head+tail of comparative compound 2, comparative compound 3, and comparative compound 4 were docked to binding site 2 (See row in Table 3). The docking scores to each of the pockets are shown in Table 3. The lower the docking score the better the ligand interaction is. When comparative compounds 2, 3, and 4 are docked to binding site 2 the scores are significantly higher which indicates that binding is less favorable.

TABLE 3
Docking scores for comparative compounds 2, 3, and 4 docked
to predicted binding site 1 and binding site 2 are shown.

| | Head | | | Head + Tail | | |
|---|---|---|---|---|---|---|
| | comp. | comp. | comp. | comp. | comp. | comp. |
| | com- | com- | com- | com- | com- | com- |
| Pocket | pound 2 | pound 3 | pound 4 | pound 2 | pound 3 | pound 4 |
| binding | −54 | −57 | −57 | −29 | −39 | −44 |
| site 1 | | | | | | |
| binding | −21 | −23 | −25 | −34 | −23 | −28 |
| site 2 | | | | | | |

Two compounds, compound 5 and compound 6, were docked to binding site 1 and binding site 2, having the structures:

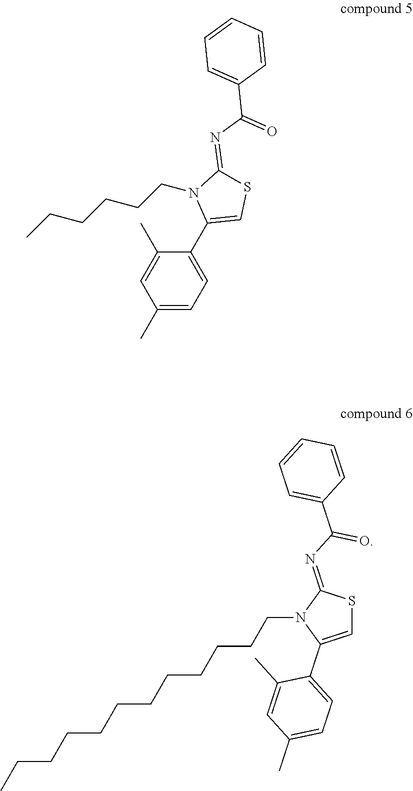 [SMILES: CCCCCCn1c(-c2ccc(C)cc2C)cs/c1=N\C(=O)c1ccccc1 ; CCCCCCCCCCCCn1c(-c2ccc(C)cc2C)cs/c1=N\C(=O)c1ccccc1]

The docking scores are shown in Table 4. Based on the results, neither compound 5 nor compound 6 appears likely to bind to binding site 1 or binding site 2.

TABLE 4
Docking score of compound 5 and compound
6 to binding site 1 and binding site 2.

| Pocket | compound 5 | compound 6 |
|---|---|---|
| binding site 1 | −13 | −5 |
| binding site 2 | −17 | −16 |

### Example 3

The effect of the indicated compounds on the synaptic density of primary mouse cortical neurons was determined after 24 hours of treatment. FIG. 6. Primary mouse cortical neurons were treated on day 15 in vitro with fascin-inhibiting small molecules at 1 uM, or with vehicle (DMSO). After 24 hours of treatment, neurons were fixed, immunolabeled for a protein component of synapses, the presynaptic vesicle protein, synaptophysin (P38), and then stained with the nuclear dye DAPI. Immunolabeled neurons were imaged on a Leica confocal microscope. The numbers of P38-immunopositive puncta were analyzed using FIJI with the Squash plugin. Data were analyzed and plotted in Graphpad Prism (** p<0.0001, * p=0.0012, 2-tailed T-test). FIG. 6 illustrates the results of synaptic growth for comparative compound 2, comparative compound 7, and compound 1 compared to vehicle control in an embodiment. Comparative compound 7 has the structure

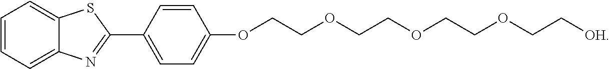 [SMILES: OCCOCCOCCOCCOc1ccc(-c2nc3ccccc3s2)cc1]

### Example 4

### Efficacy Testing Procedure

Female APOe4-TR mice are selected, because some research has shown that females with APOe4 are more likely than males to have worse memory performance, greater brain atrophy, and lower brain metabolism. Females with APOe4 are also more likely to develop mild cognitive impairment or Alzheimer's disease than males with the allele. Mice expressing the APOe3 allele (APOe3-TR), will serve as controls. In addition, a controlled cortical impact (CCI) is administered to induce reliable, calibrated TBI events in APOe4-TR and APOe3 mice. Anesthetized mice subjected to CCI will receive an impact with speed 5.0 m/s, 1.0 mm depth, and 50 ms dwell time. Sham animals will undergo the same ˜20-25 min procedure without impact.

Determination of CCI Exacerbates Cognitive Decline in APOe4-TR Mice.

It is hypothesized that cognitive and motor abilities in APOe4 mice will be worsened by the synergistic effects of a TBI and APOe4 genotype on dendritic spine synapses. To test this, CCI is administered (or no-impact sham procedure) to APOe4-TR and APOe3-TR mice at 8 months of age (12 mice per group), then behavioral testing at 10 and 12 months of age. Behavioral testing is scheduled from the least stressful to the most stressful tasks (i.e. minimal handling vs aversive tasks involving restraint) tasks, as follows (phenotyping of behavior is performed using Ethovision Xt (Noldus)). Open field test (Day I): Using video tracking, the time and frequency mice spend in the center and periphery of an arena can be used to evaluate anxiety, with increased time in the periphery indicating increased anxiety and the distance and velocity traveled indicating locomotor/spontaneous activity. Novel place and object recognition (Days 6-7): After 4 days of habituation (on day 6), mice investigate two identical objects in an arena, then on the next day (day 7), they are placed back in the arena and one of the objects is replaced with a novel object. Increased time spent investigating the familiar object in a novel place or the novel object indicates improved memory. Y-maze (Day 8): Y-maze spontaneous alternation is conducted to measure the willingness of rodents to explore new environments. Mice prefer to explore a new arm rather than returning to the arm previously visited. Each mouse is placed at the end of one arm and allowed to move freely through the maze during an 8-min session. Alternation is defined as successive entries into the three arms on overlapping triplet sets. Barnes maze (Days 9-13): This test evaluates hippocampal-dependent spatial learning and memory. During acquisition, mice are trained to locate a hidden escape hole in a circular table using extra-maze visual cues. Mice are tested on 2 trials/day with an inter-trial period of ˜30 min. If the mouse fails to enter the escape cage within 5 min, it will be guided to the correct escape location. Tail suspension (Day 14): The tail-suspension test involves suspending mice above the ground by their tails. Immobility time (sec) is used as a measure of stress. Contextual fear condition (Days 15-16): Mice are placed into a novel chamber where an electric shock is delivered Twenty-four hours later, mice are returned to the chamber and the amount of freezing behavior is recorded, with increased freezing indicating improved memory for the context.

Expected Results and Alternative Outcomes:

The effects of TBI and APOe4 genotype on synapse loss and cognition are characterized. Thus, the combination of these Alzheimer's risk factors is expected to lead to further impairment (beyond APOe4 alone) in the tasks measured, particularly spatial memory, as it relies structures that are hard hit by synapse loss during Alzheimer's and TBI. While unlikely, it is possible that no worsening of performance on any of the tasks with addition of TBI will be observed. In this case, we will consider optimizing the CCI paradigm to induce more robust synaptic loss. It is possible that some impairments will not scale with detectable synaptic loss. Addition of a TBI may make whatever impairments are in place harder to rescue, and thus provide a valid test of the ability of SPGs to treat a common gene X environment interaction in Alzheimer's disease.

Determination of Compounds as Ameliorating Cognitive Decline Due to TBI in APOe4-TR Mice.

The spinogenic effect of a compound may offset the loss of synapses induced by APOe4 genotype and the addition of TBI, leading to improved functional recovery. APOe4-TR and APOe3-TR mice subjected to CCI at 8 months of age as in Aim 1 are treated with the compound (30 mg/kg/day, i.p.) or vehicle beginning immediately after the CCI procedure and continuing through behavioral testing (as described in Aim 1) at 10 and 12 months of age. An i.p. route of administration is used for daily dosing in rodents over an extended period.

Expected Results and Alternative Outcomes:

Both cognitive and motor functions are improved in the APOe4-TR mice exposed to CCI. In some instances, only one symptom domain is rescued (e.g. spatial memory, but not motor performance) due to synergism between APOe4 status and TBI on neuronal viability in a particular brain region—beyond a certain level of neuronal death, rescue of synaptic density may not be possible. Histological data is reviewed to calibrate neuron loss in view of CCI protocol

Aim 3: Evaluate the Effect of a Compound on Dendritic Spine Density and Alzheimer's Related Molecular Biomarkers in APOe4-TR Mice.

We hypothesize that the mice treated with a compound in Aim 2 (APOe4-TR+/−CCI) will show improvements in spine density and reductions in Ab-initiated dephosphorylation of cofilin. In addition, because inhibiting fascin may reduce microglial migration and activation, we postulate that an active compound will reduce synaptic pruning by microglia. The measured attributes include (i) synaptic density, (ii) phospho-cofilin and (in) synapse-associated activated microglial. In addition, levels of phosph-tau and Ab are assessed.

Mice are anesthetized, perfused transcardially with paraformaldehyde, and brains collected for histological and dendritic spine analyses. Neuronal loss is assessed in the hippocampus, entorhinal cortex, and several neocortical areas (e.g. insula, prefrontal cortex) using Cresyl violet and NeuN stained sections via unbiased stereology (Stereo Investigator System, MBF Biosciences). Brain volumes, particularly in the hippocampal formation, are measured via Cavaliery method. Synaptic puncta will be labeled with antibodies against synaptophysin (presynaptic terminals) and PSD95 (postsynaptic density) and counted in Bitplane Imaris. Fascin levels and distribution relative to synapses are determined similarly. Glial cell densities and activation states are also assessed. Microglia are detected via immunohistochemistry for IBA1, and automated microglial cell body counts performed by Bitplane Imaris. Additional testing includes stratify microglia into synapse-associated and extra synaptic IBA+ cells based on proximity to PSD95 staining. In addition, stratify microglia as plaque-associated and non-plaque associated, IBA1+ cells. The activation state of the microglia is assessed using antibodies against a series of known microglia surface markers including CD45 and CD68. Astrocyte numbers and activation states and distribution relative to synapses are similarly assessed using antibodies against GFAP, BLPB and S100b. In addition, Aβ and tau pathology is measured using 3D volumetric Aβ/tau load analysis via Imaris software and commercially available antibodies.

Stereological quantifications are performed using Stereo-Investigator software from Microbrightfield Bioscience (MBF Bioscience, Williston, Vt., USA) to determine the number of spines in the stratum radiatum (SR) and stratum lacunosum-moleculare of the hippocampal CA3 region. Briefly, every 2nd section are used through the entire anterostereological quantifications are performed using Stereo-Investigator software from Microbrightfield Bioscience (MBF Bioscience, Williston, Vt., USA) to determine the number of spines in the stratum radiatum (SR) and stratum lacunosum-moleculare of the hippocampal CA3 region. Briefly, every 2nd section is used through the entire anterostereological quantifications are performed using Stereo-Investigator software from Microbrightfield Bioscience (MBF Bioscience, Williston, Vt., USA) to determine the number of spines in the stratum radiatum (SR) and stratum lacunosum-moleculare of the hippocampal CA3 region. Briefly, every 2nd section was used through the entire anterostereological quantifications are performed using Stereo-Investigator software from Microbrightfield Bioscience (MBF Bioscience, Williston, Vt., USA) to determine the number of spines in the stratum radiatum (SR) and stratum lacunosum-moleculare of the hippocampal CA3 region. Briefly, every 2nd section is used through the entire anter Dendritic spine density and dendritic morphology will be measured stereologically using Neurolucida software (MBF Bioscience, Williston, Vt., USA) to determine the number of spines in the stratum radiatum (sr) and dentate gyrus (dg) of the hippocampus. Mouse brains will be processed using a superGolgi Kit (Bioenno Tech LLC, Santa Ana, Calif.), as described previously. The CA1 stratum radiatum and dentate gyrus regions of hippocampus, as well as layer 2 of the entorhinal cortex and layer ⅔ of insula and medial prefrontal cortex, will be defined using a 5× objective and spines counted using a 100×/1.4 objective. Dendritic spine length and spine volume will be traced using a 100×/1.4 objective and data will be analysed via Neurolucida Explorer software. Spine density will be correlated with behavioural rescue measures of Aim 2. For dendritic morphological analysis, 5 neurons per animal (n=6) in CA1 hippocampal area will be traced using Neurolucida software and evaluated using Sholl analysis.

Expected Results and Alternative Outcomes:

Treatment increases dendritic spine density in the hippocampus by ˜20% or more, which approximately offset losses incurred by APOe4 genotype and APOe4 X CCI, and increases levels of phospho-cofilin. In some instances, treatment reduces microglial scavenging of synapses.

### Example 5

### Spinogenesis Testing

To determine if small molecule inhibitors of Fascin1 induce the formation of new dendritic spine synapses in prefrontal cortex, mice are treated with Fascin1 inhibitors compound 1 and compound 11, followed by Golgi stain analysis of dendritic spine density and morphology. Specifically, 15 C57BL/6J mice at 26 weeks of age are acclimated to housing and handling conditions for 3 days, then randomly assigned to 3 treatment groups (5 mice/group): (1) vehicle (7% DMSO, 14% Tween 80, H₂O) controls; (2) 10 mg/kg compound 1; (3) 10 mg/kg compound 11. Mice are then injected i.p. with vehicle, compound 1, or compound 11 on a daily basis for 26 days. Two hours after the final injection, mice are sacrificed and brains are rapidly removed for emersion fixation and subsequent Golgi staining. After completion of Golgi staining, sections of prefrontal cortex are cut in the coronal plane and mounted for acquisition of images using a microscope. Dendritic spine density is analyzed on Layer II/III prefrontal cortical neurons by methods described in Example 4 herein (n=X images/section, Y sections/mouse).

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. A method of promoting spinogenesis in a patient, comprising administering to a patient in need thereof a therapeutically effective amount of a compound which binds to fascin at least at binding site 2.

2. A method of promoting spinogenesis in a patient, comprising administering to a patient in need thereof a therapeutically effective amount of a compound which binds to fascin at least at binding site 3.

3. A method of promoting spinogenesis in a patient, comprising administering to a patient in need thereof a therapeutically effective amount of a compound of formula I: 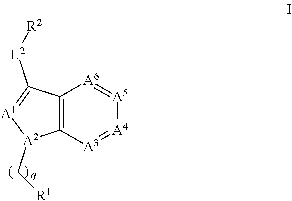
or a pharmaceutically acceptable salt thereof;
wherein A¹, A², A³, A⁴, A⁵ and A⁶ are independently selected from the group consisting of CH, CR³ and N, provided that no more than four of A¹, A², A³, A⁴, A⁵ and A⁶ are N;
R¹ is selected from the group consisting of phenyl, 5-membered heteroaryl and 6-membered heteroaryl, wherein the phenyl, 5-membered heteroaryl or 6-membered heteroaryl is optionally substituted with 1 to 3 R⁶;
L² is selected from the group consisting of a covalent bond, —NR⁸—, —C(O)NR⁸—, —NR⁸—, —C(O)NR⁸—, —NR⁸C(O)—, —C(O)CR⁸₂—, —CR⁸₂C(O)—, —NR⁸CR⁸₂—, and —CR⁸₂NR⁸—;
R² is H, C₁₋₆ alkyl, 6- to 10-membered aryl or 5- to 10-membered heteroaryl; wherein the 6- to 10-membered aryl or 5- to 10-membered heteroaryl is optionally substituted with 1 to 4 R⁴, wherein each R⁴ is independently selected from the group consisting of C₁₋₆ alkyl, C₁₋₆ haloalkyl, phenyl (optionally substituted with C₁₋₆ alkyl, halo, C₁₋₆ haloalkyl, or —OH), —OH, —OR⁷, —SH, —SR⁷, —NR¹⁰R¹⁰, halo, cyano, nitro, —COH, —COR⁷, —CO₂H, —CO₂R⁷, —CONR¹⁰R¹⁰, —OCOR⁷, —OCO₂R⁷, —OCONR¹⁰R¹⁰, —NR¹⁰COR⁷, —NR¹⁰CO₂R⁷, —SOR⁷, —SO₂R⁷, —SO₂NR¹⁰R¹⁰, and —NR¹⁰SO₂R⁷;
each R³ is independently selected from the group consisting of C₁₋₆ alkyl, C₁₋₆ haloalkyl, —OH, —OR⁷, —SH, —SR⁷, —NR¹⁰R¹⁰, halo, cyano, nitro, —COH, —COR⁷, —CO₂H, —CO₂R⁷, —CONR¹⁰R¹⁰, —OCOR⁷, —OCO₂R⁷, —OCONR¹⁰R¹⁰, —NR¹⁰COR⁷, —NR¹⁰CO₂R⁷, —SOR⁷, —SO₂R⁷, —SO₂NR¹⁰R¹⁰, and —NR¹⁰SO₂R⁷;
q is 1, 2 or 3;
each R⁶ is independently selected from the group consisting of cyano, halo, C₁₋₆ alkyl, C₁₋₆ haloalkyl, and —CH₂OH;
R⁷ is C₁₋₆ alkyl or C₁₋₆ haloalkyl;
R⁸ is hydrogen or C₁₋₆ alkyl;
each R¹⁰ is independently hydrogen or C₁₋₆ alkyl, or two R¹⁰ together with the atom(s) attached thereto form a 4- to 6-membered ring; and
R¹¹ is hydrogen or R³.

4. A method of promoting spinogenesis in a patient, comprising administering to a patient in need thereof a therapeutically effective amount of a compound selected from a compound of formula II, formula IV, formula V, formula VII, formula VIII, formula IX, formula X, or formula XI, or a pharmaceutically acceptable salt thereof, or compound 1, compound 8, compound 9, compound 10, or compound 11, or a pharmaceutically acceptable salt thereof.

5. A method of treating or preventing a neuronal disease or disorder comprising administering to a patient in need thereof, a therapeutically effective amount of a compound which binds to fascin at least at binding site 2.

6. A method of treating or preventing a neuronal disease or disorder comprising administering to a patient in need thereof, a therapeutically effective amount of a compound which binds to fascin at least at binding site 3.

7. The method of claim 5 or claim 6, wherein the compound is selected from a compound of formula I, formula II, formula IV, formula V, formula VII, formula VIII, formula IX, formula X, or formula XI, or a pharmaceutically acceptable salt thereof, or compound 1, compound 8, compound 9, compound 10, or compound 11, or a pharmaceutically acceptable salt thereof.

8. A method of treating or preventing a neuronal disease or disorder comprising administering to a patient in need thereof a therapeutically effective amount of a compound of formula I: 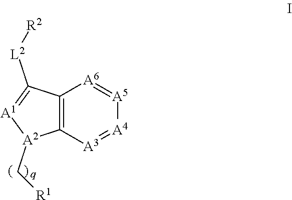
or a pharmaceutically acceptable salt thereof;
wherein A¹, A², A³, A⁴, A⁵ and A⁶ are independently selected from the group consisting of CH, CR³ and N, provided that no more than four of A¹, A², A³, A⁴, A⁵ and A⁶ are N;
R¹ is selected from the group consisting of phenyl, 5-membered heteroaryl and 6-membered heteroaryl, wherein the phenyl, 5-membered heteroaryl or 6-membered heteroaryl is optionally substituted with 1 to 3 R⁶;
L² is selected from the group consisting of a covalent bond, —NR⁸—, —C(O)NR⁸—, —NR⁸—, —C(O)NR⁸—, —NR⁸C(O)—, —C(O)CR⁸₂—, —CR⁸₂C(O)—, —NR⁸CR⁸₂—, and —CR⁸₂NR⁸—;
R² is H, C₁₋₆ alkyl, 6- to 10-membered aryl or 5- to 10-membered heteroaryl; wherein the 6- to 10-membered aryl or 5- to 10-membered heteroaryl is optionally substituted with 1 to 4 R⁴, wherein each R⁴ is independently selected from the group consisting of C₁₋₆ alkyl, C₁₋₆ haloalkyl, phenyl (optionally substituted with C₁₋₆ alkyl, halo, C₁₋₆ haloalkyl, or —OH), —OH, —OR⁷, —SH, —SR⁷, —NR¹⁰R¹⁰, halo, cyano, nitro, —COH, —COR⁷, —CO₂H, —CO₂R⁷, —CONR¹⁰R¹⁰, —OCOR⁷, —OCO₂R⁷, —OCONR¹⁰R¹⁰, —NR¹⁰COR⁷, —NR¹⁰CO₂R⁷, —SOR⁷, —SO₂R⁷, —SO₂NR¹⁰R¹⁰, and —NR¹⁰SO₂R⁷;
each R³ is independently selected from the group consisting of C₁₋₆ alkyl, C₁₋₆ haloalkyl, —OH, —OR⁷, —SH, —SR⁷, —NR¹⁰R¹⁰, halo, cyano, nitro, —COH, —COR⁷, —CO₂H, —CO₂R⁷, —CONR¹⁰R¹⁰, —OCOR⁷, —OCO₂R⁷, —OCONR¹⁰R¹⁰, —NR¹⁰COR⁷, —NR¹⁰CO₂R⁷, —SOR⁷, —SO₂R⁷, —SO₂NR¹⁰R¹⁰, and —NR¹⁰SO₂R⁷;
q is 1, 2 or 3;
each R⁶ is independently selected from the group consisting of cyano, halo, C₁₋₆ alkyl, C₁₋₆ haloalkyl, and —CH₂OH;
R⁷ is C₁₋₆ alkyl or C₁₋₆ haloalkyl;
R⁸ is hydrogen or C₁₋₆ alkyl;
each R¹⁰ is independently hydrogen or C₁₋₆ alkyl, or two R¹⁰ together with the atom(s) attached thereto form a 4- to 6-membered ring; and
R¹¹ is hydrogen or R³.

9. The method of any one of claims 5-7, wherein the neuronal disease or disorder is selected from Alzheimer's disease, Parkinson's disease, Parkinson's dementia, autism, fragile X syndrome, depression, and traumatic brain injury.

10. The method of any one of claims 5-7, wherein the neuronal disease or disorder is Alzheimer's disease.

11. The method of any one of claims 5-7, wherein the neuronal disease or disorder is depression.

12. A method of promoting spinogenesis in a patient, comprising administering to a patient in need thereof a therapeutically effective amount of N-(1-(4-(trifluoromethyl)benzyl)-1H-indazol-3-yl)furan-2-carboxamide (compound 1), having the structure: 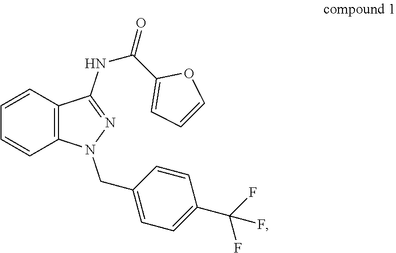 [SMILES: O=C(Nc1nn(Cc2ccc(C(F)(F)F)cc2)c2ccccc12)c1ccco1]
or a pharmaceutically acceptable salt thereof.

13. A method of treating a depression in a patient in need thereof comprising administering to the patient a therapeutically effective amount of a compound selected from a compound of formula II, formula IV, formula V, formula VII, formula VIII, formula IX, formula X, or formula XI, or a pharmaceutically acceptable salt thereof, or compound 1, compound 8, compound 9, compound 10, or compound 11, or a pharmaceutically acceptable salt thereof.

14. A method of treating depression comprising administering to a patient in need thereof a therapeutically effective amount of a compound of formula I: 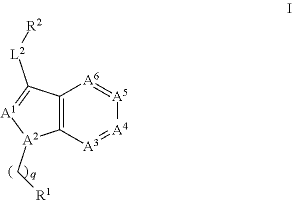
or a pharmaceutically acceptable salt thereof;
wherein A¹, A², A³, A⁴, A⁵ and A⁶ are independently selected from the group consisting of CH, CR³ and N, provided that no more than four of A¹, A², A³, A⁴, A⁵ and A⁶ are N;
R¹ is selected from the group consisting of phenyl, 5-membered heteroaryl and 6-membered heteroaryl, wherein the phenyl, 5-membered heteroaryl or 6-membered heteroaryl is optionally substituted with 1 to 3 R⁶;
L² is selected from the group consisting of a covalent bond, —NR⁸—, —C(O)NR⁸—, —NR⁸—, —C(O)NR⁸—, —NR⁸C(O)—, —C(O)CR⁸₂—, —CR⁸₂C(O)—, —NR⁸CR⁸₂—, and —CR⁸₂NR⁸—;
R² is H, C₁₋₆ alkyl, 6- to 10-membered aryl or 5- to 10-membered heteroaryl; wherein the 6- to 10-membered aryl or 5- to 10-membered heteroaryl is optionally substituted with 1 to 4 R⁴, wherein each R⁴ is independently selected from the group consisting of C₁₋₆ alkyl, C₁₋₆ haloalkyl, phenyl (optionally substituted with C₁₋₆ alkyl, halo, C₁₋₆ haloalkyl, or —OH), —OH, —OR⁷, —SH, —SR⁷, —NR¹⁰R¹⁰, halo, cyano, nitro, —COH, —COR⁷, —CO₂H, —CO₂R⁷, —CONR¹⁰R¹⁰, —OCOR⁷, —OCO₂R⁷, —OCONR¹⁰R¹⁰, —NR¹⁰COR⁷, —NR¹⁰CO₂R⁷, —SOR⁷, —SO₂R⁷, —SO₂NR¹⁰R¹⁰, and —NR¹⁰SO₂R⁷;
each R³ is independently selected from the group consisting of C₁₋₆ alkyl, C₁₋₆ haloalkyl, —OH, —OR⁷, —SH, —SR⁷, —NR¹⁰R¹⁰, halo, cyano, nitro, —COH, —COR⁷, —CO₂H, —CO₂R⁷, —CONR¹⁰R¹⁰, —OCOR⁷, —OCO₂R⁷, —OCONR¹⁰R¹⁰, —NR¹⁰COR⁷, —NR¹⁰CO₂R⁷, —SOR⁷, —SO₂R⁷, —SO₂NR¹⁰R¹⁰, and —NR¹⁰SO₂R⁷;
q is 1, 2 or 3;
each R⁶ is independently selected from the group consisting of cyano, halo, C₁₋₆ alkyl, C₁₋₆ haloalkyl, and —CH₂OH;
R⁷ is C₁₋₆ alkyl or C₁₋₆ haloalkyl;
R⁸ is hydrogen or C₁₋₆ alkyl;
each R¹⁰ is independently hydrogen or C₁₋₆ alkyl, or two R¹⁰ together with the atom(s) attached thereto form a 4- to 6-membered ring; and
R¹¹ is hydrogen or R³.
